(19) United States
(12) Patent Application Publication
(10) Pub. No.: US 2024/0343706 A1
(43) Pub. Date: Oct. 17, 2024

(54) PIPERIDINE-2,6-DIONE DERIVATIVES WHICH BIND TO CEREBLON, AND METHODS OF USE THEREOF

(71) Applicant: Captor Therapeutics S.A., Wroclaw (PL)

(72) Inventors: Katarzyna KACZANOWSKA, Wroclaw (PL); Sylvain COTTENS, Witterswil (CH); Roman PLUTA, Krakow (PL); Niall DICKINSON, Skierniewice (PL); Michal WALCZAK, Wroclaw (PL)

(21) Appl. No.: 17/780,891

(22) PCT Filed: Nov. 27, 2020
(86) PCT No.: PCT/EP2020/083596

§ 371 Date: May 27, 2022

(30) Foreign Application Priority Data
Nov. 27, 2019 (WO) PCT/PL2019/000109

(51) Int. Cl. C07D 401/12; A61K 47/55; C07D 403/12; C07D 405/12; C07D 405/14; C07D 409/12; C07D 409/14; C07D 413/12; C07D 417/12; C07D 471/04; C07D 495/04; C07D 513/04

(52) U.S. Cl. CPC C07D 401/12; A61K 47/55; C07D 403/12; C07D 405/12; C07D 405/14; C07D 409/12; C07D 409/14; C07D 413/12; C07D 417/12; C07D 471/04; C07D 495/04; C07D 513/04

(57) ABSTRACT

The present invention provides novel compounds which bind to cereblon, and methods of use thereof. The compounds are represented by Formulas (I) and (II), below: (I) wherein R^{x} is selected from (Ia), (Ib), (Ic) and (Id); (II) wherein R^{y} is selected from (IIa), (IIb), (IIc) and (IId).

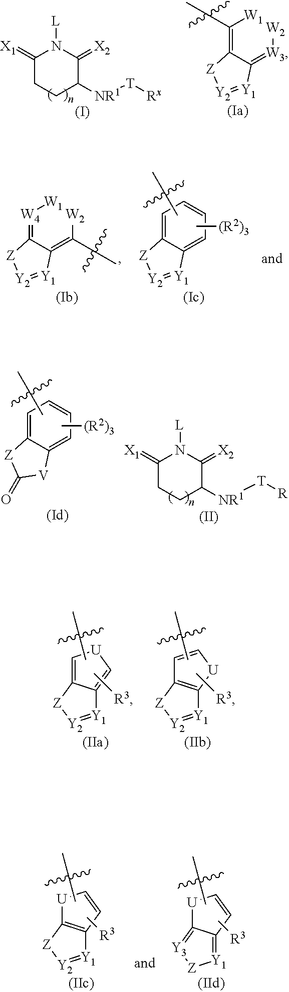

### CROSS-REFERENCE TO RELATED APPLICATIONS

This application is a U.S. National Stage Application under 35 U.S.C. § 371 of International Application No. PCT/EP2020/083596, filed Nov. 27, 2020, which claims priority to Patent Application No. PCT/PL2019/000109 (PL), filed Nov. 27, 2019, the contents of each of which are incorporated herein by reference in their entireties.

### FIELD OF THE INVENTION

The present invention relates to novel compounds which bind to the protein cereblon and modulate the substrate specificity of CUL4-DDB1-RBX1-CRBN ubiquitin ligase complex (CRL4^{CRBN}). Cereblon is a substrate recognition component of CRL4^{CRBN}. Chemical modulation of cereblon may induce association of novel substrate proteins, followed by their ubiquitination and degradation. The present invention also provides bifunctional compounds, which contain a ligand which binds to the cereblon E3 ubiquitin ligase and a moiety which binds a target protein such that the target protein is placed in proximity to the ubiquitin ligase to induce degradation of that protein.

### BACKGROUND

Cereblon (CRBN) is a protein which associates with DDB1 (damaged DNA binding protein 1), CUL4 (Cullin-4), and RBX1 (RING-Box Protein 1). Collectively, the proteins form a ubiquitin ligase complex, which belongs to Cullin RING Ligase (CRL) protein family and is referred to as CRL4^{CRBN}. Cereblon became of particular interest to the scientific community after it was confirmed to be a direct protein target of thalidomide, which mediates the biological activity of cereblon. Thalidomide, a drug approved for treatment of multiple myeloma in the late 1990s, binds to cereblon and modulates the substrate specificity of the CRL4^{CRBN} ubiquitin ligase complex. This mechanism underlies the pleiotropic effect of thalidomide on both immune cells and cancer cells (see Lu G et al.: The Myeloma Drug Lenalidomide Promotes the Cereblon-Dependent Destruction of Ikaros Proteins. Science. 2014 Jan. 17; 343(6168): 305-9).

Thalidomide's success in cancer therapy stimulated efforts towards development of analogues with higher potency and fewer detrimental side effects. As a result, various drug candidates were produced: lenalidomide, pomalidomide, CC-220, CC-122, CC-885, and TD-106. These compounds are collectively called Cereblon Modulating Agents (CMAs). For discussions of these compounds, see—for example—U.S. Pat. No. 5,635,517(B2), WO2008039489 (A2), WO2017197055 (A1), WO2018237026 (A1), WO2017197051 (A1), U.S. Pat. No. 8,518,972 (B2), EP 2057143 (B1), WO2019014100 (A1), WO2004103274 (A2), and Kim S A et al.: A novel cereblon modulator for targeted protein degradation. Eur J Med Chem. 2019 Mar. 15; 166: 65-74.

The clinical applicability of CMAs in numerous hematologic malignancies, such as multiple myeloma, myelodysplastic syndromes lymphomas and leukemia, has been demonstrated (see Le Roy A et al.: Immunomodulatory Drugs Exert Anti-Leukemia Effects in Acute Myeloid Leukemia by Direct and Immunostimulatory Activities. Front Immunol. 2018; 9: 977).

The antitumor activity of cereblon modulators is mediated by:
1) inhibition of cancer cell proliferation and induction of apoptosis,
2) disruption of trophic support from tumor stroma,
3) stimulation of immune cells, resulting in proliferation of T-cells, cytokine production and activation of NK (natural killer) cells (see Le Roy A et al.: Immunomodulatory Drugs Exert Anti-Leukemia Effects in Acute Myeloid Leukemia by Direct and Immunostimulatory Activities. Front Immunol. 2018; 9: 977).

It has been demonstrated that chemically-modified thalidomide-based derivatives can significantly modify the substrate specificity of CRL4^{CRBN} ubiquitin ligase. Thus, it is desired to progress development of cereblon modulating agents in order to achieve desired substrate specificity in the CMA-bound CRL4^{CRBN} ubiquitin ligase complex (see Sievers Q L et al.: Defining the human C₂H₂ zinc finger degrome targeted by thalidomide analogues through CRBN. Science. 2018 Nov. 2; 362(6414)) to reach a desired safety profile. There is thus a continuing need to provide novel cereblon-binding compounds which have pharmaceutically relevant properties.

Alternatively, chemically-modified thalidomide-based derivatives can be linked to a target protein binding ligand to form bifunctional compounds. Such compounds, upon addition to cells or administration to an animal or human, are capable of inducing proteasome-mediated degradation of selected proteins via their recruitment to cereblon and subsequent ubiquitination. This concept was first described by Sakamoto K M et al.: Chimeric molecules that target proteins to the Skp1-Cullin-F box complex for ubiquitination and degradation. Proc Natl Acad Sci USA. 2001 Jul. 17; 98(15):8554-9 and more recently reviewed by Burslem G M and Crews C M: Proteolysis-Targeting Chimeras as Therapeutics and Tools for Biological Discovery. Cell. 2020 Apr. 2; 181(1):102-114.

Thalidomide derivatives applied in the design of cereblon-recruiting bifunctional compounds, such as pomalidomide and lenalidomide, induce degradation of various neosubstrates, such as IKZF1, IKZF3, SALL4 and/or CK1α. Thus, treatment with bifunctional compounds built of these known CMAs results not only in the degradation of a selected target protein, but in a degradation of additional proteins induced by the CRBN ligands themselves, which may lead to various side effects. Side effects resulting from lenalidomide activity include neutropenia, thrombocytopenia, and hemorrhagic disorders (see: Sun X et al. PROTACs: great opportunities for academia and industry. Signal Transduct Target Ther. 2019 Dec. 24; 4:64 and Stahl M, Zeidan A M: Lenalidomide Use in Myelodysplastic Syndromes: Insights Into the Biologic Mechanisms and Clinical ApplicationsCancer. 2017 May 15; 123(10):1703-1713).

### SUMMARY OF INVENTION

In accordance with a first aspect of the invention, there is provided a compound of Formula (I):

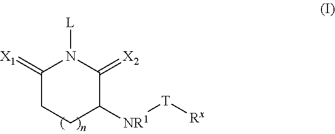

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L is hydrogen, alkenyl, aryl, heteroaryl, benzyl, haloalkyl, haloalkenyl, —C(O)H, —C(O)R″, —C(O)OH, —C(O)OR″, —CH₂C(O)OR″, —C(O)NH₂, —C(O)NHR″, —C(O)NR″₂, —OH, —OR″, —NH₂, —NHR″, —NR″₂, —S(O)₂H or —S(O)₂R″;
R^{x} is selected from

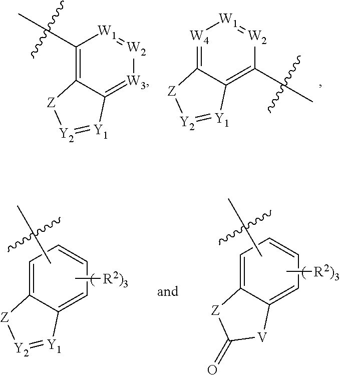

wherein [symbol] indicates attachment to T,
Z is O, S or NR⁴;
V is CR₂, NR⁴ or S;
each of W₁, W₂, W₃ and W₄ is independently N or CR²,
each of Y₁ and Y₂ is independently N or CR,
each R is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂; or when Y₁ and Y₂ are CR then each R, together with the carbon atom to which it is attached, forms a 5- or 6-membered ring;
each R² is independently hydrogen, halogen, alkyl, cycloalkyl, alkenyl, cycloalkenyl, alkynyl, aryl, aryl substituted with at least one —OR″, heteroaryl, benzyl, haloalkyl, haloalkenyl, —NH₂, —NHR″, —NR″₂, —CH₂NH₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂;
R⁴ is hydrogen, halogen, alkyl, cycloalkyl, alkenyl, cycloalkenyl, alkynyl, aryl, heteroaryl, benzyl, haloalkyl, haloalkenyl, —C(O)H, C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H or —S(O)₂R″; and
each R″ is independently alkyl, cycloalkyl, alkenyl, cycloalkenyl, aryl, heteroaryl, or benzyl;
wherein, when n=2, each R² is hydrogen, and each of W₁, W₂, W₃ and W₄ is CR², then C═X₁ may be replaced by CH;
and wherein:
(i) when R^{x} is

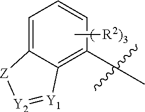

and Z is NH, then L is hydrogen, —CH₂C(O)OR″, or —OR″.
(ii) when R^{x} is

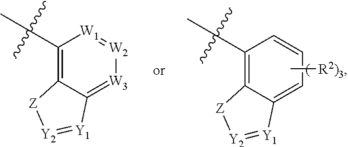

Z is NR⁴, Y₁ is CR, and Y₂ is N, then R⁴ is not alkyl and at least one of R² and R is not H;
(iii) when R^{x} is

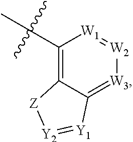

Z is NR⁴, and Y₁ and Y₂ are CR, then at least one of W₁, W₂ and W₃ is N;
(iv) when Z is NR⁴, and Y₁ and Y₂ are CR, then R^{x} is not
(v) when R^{x} is

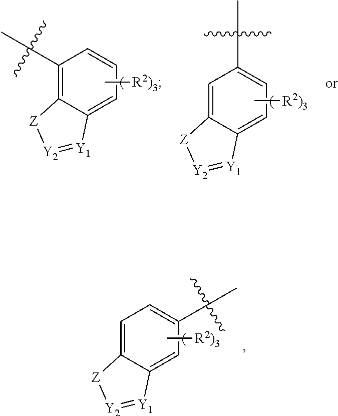

Z is NR⁴, and Y₁ or Y₂ is N, then R⁴ is not alkyl;
(vi) when R^{x} is

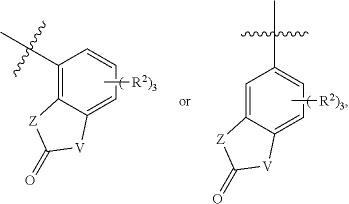

then n=1 or 2; and
(vii) when R^{x} is

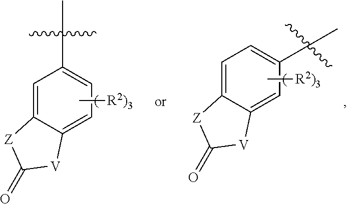

then Z═O or S

In some embodiments, the compound of Formula (I) has the structure:

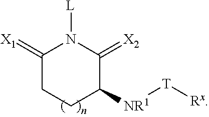

In other embodiments, the compound of Formula (I) has the structure:

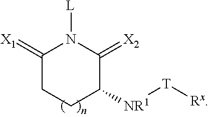

In some embodiments of the compound of Formula (I), T is C═O. In other embodiments, T is SO₂.

In some embodiments of the compound of Formula (I), Z is NR⁴. In some embodiments of the compound of Formula (I), Z is NH. In other embodiments, Z is O. In other embodiments, Z is S.

In some embodiments of the compound of Formula (I), V is CR₂. In other embodiments, V is NR⁴. In other embodiments, V is S.

In some embodiments of the compound of Formula (I), Y₁ is N, and Y₂ is CR. In other embodiments, Y₂ is N, and Y₁ is CR.

In some embodiments of the compound of Formula (I), both of Y₁ and Y₂ are N. In other embodiments, both of Y₁ and Y₂ are CR.

In some embodiments of the compound of Formula (I), L is hydrogen, alkenyl, aryl, heteroaryl, benzyl, haloalkyl, haloalkenyl, —OH, —OR″, —CH₂C(O)OR″, —NH₂, —NHR″, —NR″₂, —S(O)₂H or —S(O)₂R″. In other embodiments of the compound of Formula (I), L is hydrogen alkenyl, aryl, heteroaryl, benzyl, haloalkyl, haloalkenyl, —C(O)R″, —C(O)OR″, —CH₂C(O)OR″, —C(O)NH₂, —C(O)NHR″, or —C(O)NR″₂. In some embodiments of the compound of Formula (I), L is hydrogen, alkenyl, aryl, heteroaryl, benzyl, haloalkyl, haloalkenyl, —OH, —OR″, —CH₂C(O)OR″, —NH₂, —NHR″, —NR″₂, —S(O)₂H or —S(O)₂R″. In some embodiments of the compound of Formula (I), L is hydrogen, alkenyl, aryl, heteroaryl, benzyl, haloalkyl, or haloalkenyl. In other embodiments of the compound of Formula (I), L is —OH, —OR″, —CH₂C(O)OR″, —NH₂, —NHR″, —NR″₂, —S(O)₂H or —S(O)₂R″. In some embodiments of the compound of Formula (I), L is hydrogen, alkenyl, aryl, heteroaryl, or benzyl. In some embodiments of the compound of Formula (I), L is hydrogen, alkenyl, or aryl. In some embodiments of the compound of Formula (I), L is hydrogen, or alkenyl. In some embodiments of the compound of Formula (I), L is hydrogen, —CH₂C(O)OR″ or —OR″. In some embodiments of the compound of Formula (I), L is hydrogen.

In some embodiments of the compound of Formula (I), R^{x} is selected from

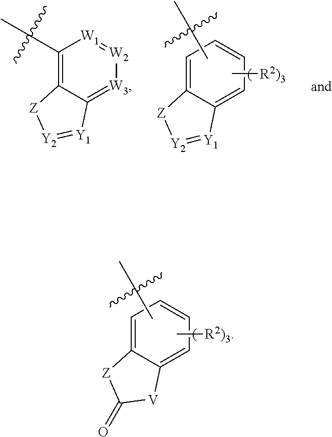

In some embodiments of the compound of Formula (I), R^{x} is selected from

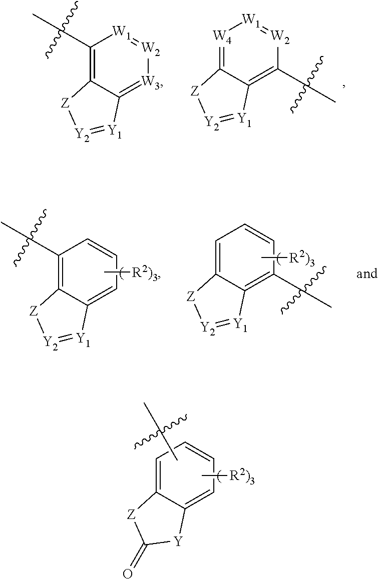

In some embodiments of the compound of Formula (I), R^{x} is selected from

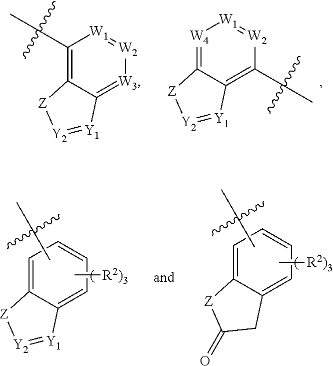

In some embodiments of the compound of Formula (I), R^{x} is

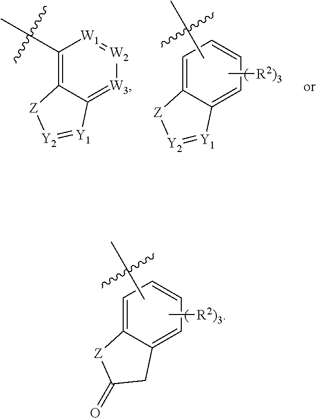

In some embodiments of the compound of Formula (I), R^{x} is selected from

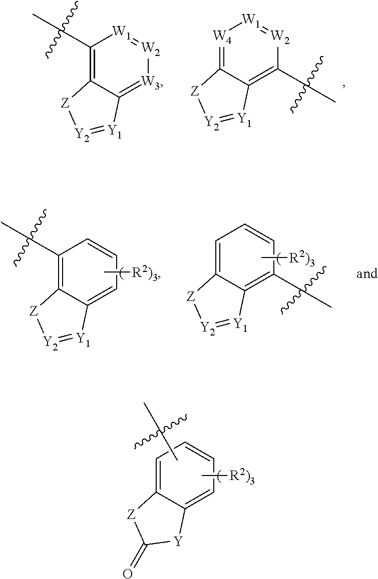

In some embodiments of the compound of Formula (I), R^{x} is selected from

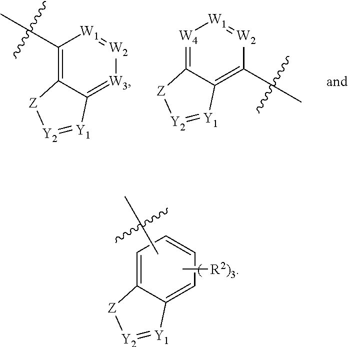

In some embodiments of the compound of Formula (I), R^{x} is selected from

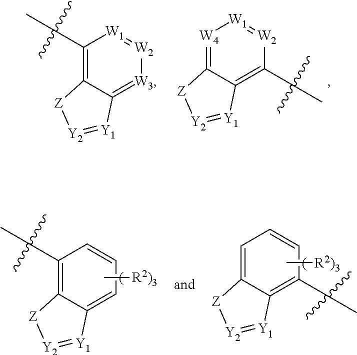

In some embodiments of the compound of Formula (I), R^{x} is

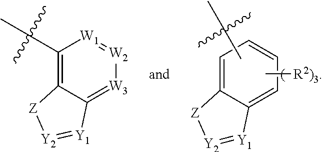

In some embodiments of the compound of Formula (I), R^{x} is selected from

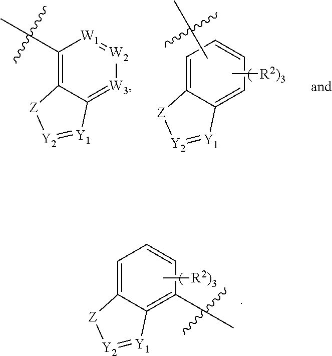

In some embodiments of the compound of Formula (I), R^{x} is

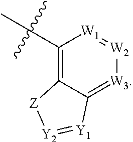

In some such embodiments, one of W₁, W₂ and W₃ is N, and the remaining two of W₁, W₂ and W₃ are each CR². In some embodiments, W₁ is N, and W₂ and W₃ are each CR². In other embodiments, W₂ is N, and W₁ and W₃ are each CR². In other embodiments, W₃ is N, and W₁ and W₂ are each CR².

In other such embodiments, two of W₁, W₂ and W₃ are N, and the remaining one of W₁, W₂ and W₃ is CR². In some embodiments, W₁ and W₂ are each N, and W₃ is CR². In other embodiments, W₁ and W₃ are each N, and W₂ is CR². In other embodiments, W₂ and W₃ are each N, and W₁ is CR².

In other embodiments, each of W₁, W₂ and W₃ is N.

In other embodiments, each of W₁, W₂ and W₃ is CR².

In some such embodiments, each R² is hydrogen; Y₁ is N; and Y₂ is CH. In some such embodiments, the compound of Formula (I) has the structure:

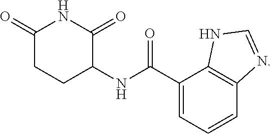 [SMILES: O=C1CCC(NC(=O)c2cccc3nc[nH]c23)C(=O)N1]

In other such embodiments, each R² is hydrogen; and Y₁ and Y₂ are each CH.

In some embodiments of the compound of Formula (I), R^{x} is

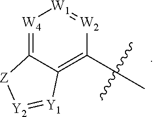

In some such embodiments, one of W₁, W₂ and W₄ is N, and the remaining two of W₁, W₂ and W₃ are each CR². In some embodiments, W₁ is N, and W₂ and W₄ are each CR². In other embodiments, W₂ is N, and W₁ and W₄ are each CR². In other embodiments, W₄ is N, and W₁ and W₂ are each CR².

In other such embodiments, two of W₁, W₂ and W₄ are N, and the remaining one of W₁, W₂ and W₃ is CR². In some embodiments, W₁ and W₂ are each N, and W₄ is CR². In other embodiments, W₁ and W₄ are each N, and W₂ is CR². In other embodiments, W₂ and W₄ are each N, and W₁ is CR².

In other such embodiments, each of W₁, W₂ and W₄ is N.

In other such embodiments, each of W₁, W₂ and W₄ is CR².

In some embodiments of the compound of Formula (I), R^{x} is

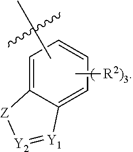

In some such embodiments, R^{x} is

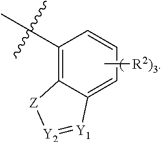

In other such embodiments, R^{x} is

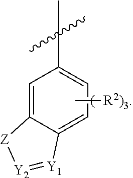

In other such embodiments, R^{x} is

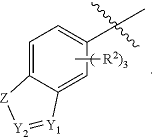

In other such embodiments, R^{x} is

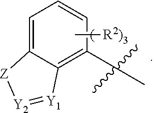

In some embodiments of the compound of Formula (I), R^{x} is

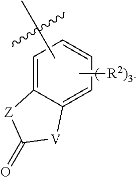

In some such embodiments, R^{x} is

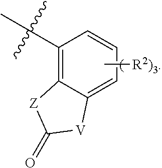

In other such embodiments, R^{x} is

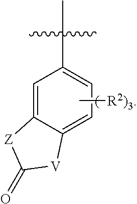

In other such embodiments, R^{x} is

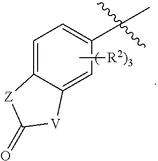

In other such embodiments, R^{x} is

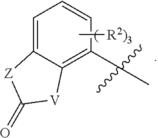

In some embodiments, R⁴ is hydrogen, halogen, alkyl, cycloalkyl, alkenyl, cycloalkenyl, alkynyl, aryl, heteroaryl, benzyl, haloalkyl, haloalkenyl, —OH, —OR″, —NH₂, —NHR″, —NR″₂, —S(O)₂H or —S(O)₂R″. In other embodiments, R⁴ is hydrogen, halogen, alkyl, cycloalkyl, alkenyl, cycloalkenyl, alkynyl, aryl, heteroaryl, benzyl, haloalkyl, haloalkenyl, —C(O)H, C(O)R″, —C(O)OH, —C(O)OR″, —C(O)NH₂, —C(O)NHR″ or —C(O)NR″₂. In some embodiments, R⁴ is hydrogen, halogen, alkyl, cycloalkyl, alkenyl, cycloalkenyl, alkynyl, aryl, heteroaryl, or benzyl. In other embodiments, R⁴ is —OH, —OR″, —NH₂, —NHR″, —NR″₂, —S(O)₂H or —S(O)₂R″. In some embodiments, R⁴ is hydrogen, alkyl, alkenyl, or aryl. In some embodiments, R⁴ is hydrogen, alkyl or alkenyl. In some embodiments, R⁴ is hydrogen or alkyl. In some embodiments, R⁴ is hydrogen.

In some embodiments, V is CH₂. In some embodiments, each R² is hydrogen and Z is NH.

In some such embodiments, the compound has the structure:

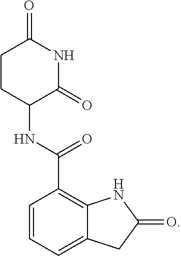 [SMILES: O=C1CCC(NC(=O)c2cccc3c2NC(=O)C3)C(=O)N1]

In some embodiments of the compound of Formula (I), each R² is independently hydrogen, halogen, alkyl, cycloalkyl, alkenyl, cycloalkenyl, alkynyl, aryl, aryl substituted with at least one —OR″, benzyl, haloalkyl, haloalkenyl, —NH₂, —NHR″, —NR″₂, —CH₂NH₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂.

In some embodiments of the compound of Formula (I), each R² is independently hydrogen, halogen, alkyl, —NH₂, —NHR″, —NHC(O)R″, —NHSO₂R″, —CN, —OH, —OR″, —S(O)₂NH₂, —S(O)₂NHR″, or —S(O)₂NR″₂.

In some embodiments of the compound of Formula (I), each R² is independently hydrogen, halogen, aryl, aryl substituted with at least one —OR″, —NH₂, —CH₂NH₂, —NHC(O)R″, —NO₂, or —OR″.

In some embodiments of the compound of Formula (I), each R² is independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂.

In some embodiments of the compound of Formula (I), each R² is hydrogen.

In some embodiments of the compound of Formula (I), when n=2 and C═X₁ is replaced by CH, then R^{x} is

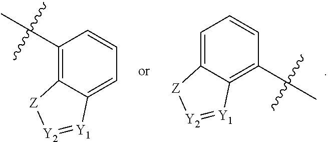

In some embodiments of the compound of Formula (I), each R is independently hydrogen, halogen, alkyl, haloalkyl, fused aryl-cycloalkyl, fused aryl-heterocycloalkyl, heteroaryl, heteroaryl substituted with at least one aryl group, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂; or when Y₁ and Y₂ are CR then each R, together with the carbon atom to which it is attached, forms a 5- or 6-membered ring.

In some embodiments of the compound of Formula (I), each R is independently hydrogen, halogen, alkyl, haloalkyl, fused aryl-cycloalkyl, fused aryl-heterocycloalkyl, heteroaryl, heteroaryl substituted with at least one aryl group, —NH₂ or —CN; or when Y₁ and Y₂ are CR then each R, together with the carbon atom to which it is attached, forms a 5- or 6-membered ring. In some embodiments, each R is hydrogen.

In some embodiments of the compound of Formula (I), R¹ is hydrogen or alkyl. In some embodiments, R¹ is hydrogen or methyl. In some embodiments, R¹ is hydrogen.

In some embodiments of the compound of Formula (I), R⁴ is hydrogen or alkyl. In some embodiments R⁴ is hydrogen or methyl; further optionally In some embodiments, R⁴ is hydrogen.

In accordance with a second aspect of the invention, there is provided a compound of Formula (II):

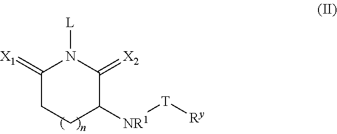

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L is hydrogen, alkyl, alkenyl, aryl, heteroaryl, benzyl, haloalkyl, haloalkenyl, —C(O)H, —C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H or —S(O)₂R″;
R^{y} is selected from

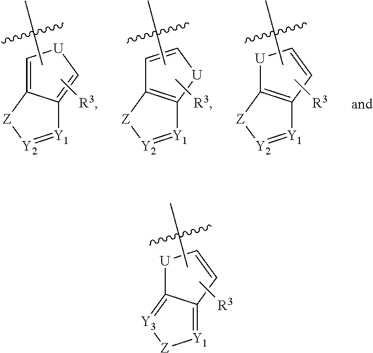

wherein [symbol] indicates attachment to T,
Z is O, S or NR³;
U is O, S, NR³ or CR²²;
each of Y₁, Y₂ and Y₃ is independently N or CR;
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂;
each R² is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR′2;
each R″ is independently alkyl, cycloalkyl, alkenyl, cycloalkenyl, aryl, heteroaryl, or benzyl;
wherein,
(i) when R^{y} is

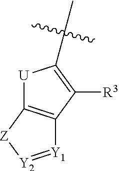

then Y₂ is CR; and
(ii) when R^{y} is

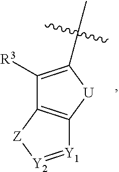

then R³ is not hydrogen.

In some embodiments, the compound of Formula (II) has the structure:

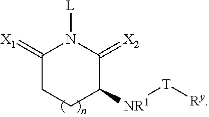

In other embodiments, the compound of Formula (II) has the structure:

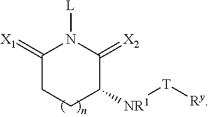

In some embodiments of the compound of Formula (II), T is C═O. In other embodiments, T is SO₂.

In some embodiments of the compound of Formula (II), Z is NR³. In other embodiments, Z is O. In other embodiments, Z is S.

In some embodiments of the compound of Formula (II), Y₁ is N, and Y₂ is CR. In other embodiments, Y₂ is N, and Y₁ is CR.

In some embodiments of the compound of Formula (II), both of Y₁ and Y₂ are N.

In some embodiments of the compound of Formula (II), both of Y₁ and Y₂ are CR.

In some embodiments of the compound of Formula (II), L is hydrogen, alkyl, alkenyl, aryl, heteroaryl, benzyl, haloalkyl, haloalkenyl, —OH, —OR″, —NH₂, —NHR″, —NR″₂, —S(O)₂H or —S(O)₂R″. In other embodiments of the compound of Formula (II), L is hydrogen, alkyl, alkenyl, aryl, heteroaryl, benzyl, haloalkyl, haloalkenyl, —C(O)R″, —C(O)OR″, —C(O)NH₂, —C(O)NHR″, or —C(O)NR″₂. In some embodiments of the compound of Formula (II), L is hydrogen, alkyl, alkenyl, aryl, heteroaryl, benzyl, haloalkyl, haloalkenyl, —OH, —OR″, —NH₂, —NHR″, —NR″₂, —S(O)₂H or —S(O)₂R″. In some embodiments of the compound of Formula (II), L is hydrogen, alkyl, alkenyl, aryl, heteroaryl, benzyl, haloalkyl, or haloalkenyl.

In other embodiments of the compound of Formula (II), L is —OH, —OR″, —NH₂, —NHR″, —NR″₂, —S(O)₂H or —S(O)₂R″. In some embodiments of the compound of Formula (II), L is hydrogen, alkyl, alkenyl, aryl, heteroaryl, or benzyl. In some embodiments of the compound of Formula (II), L is hydrogen, alkyl, alkenyl, or aryl. In some embodiments of the compound of Formula (II), L is hydrogen, alkyl, or alkenyl.

In some embodiments of the compound of Formula (II), L is hydrogen or alkyl. In some embodiments of the compound of Formula (II), L is hydrogen.

In some embodiments of the compound of Formula (II), R^{y} is

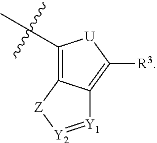

In other embodiments of the compound of Formula (II), R^{y} is

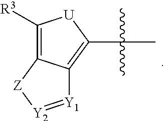

In other embodiments of the compound of Formula (II), R^{y} is

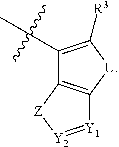

In some embodiments of the compound of Formula (II), R^{y} is

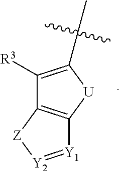

In some embodiments of the compound of Formula (II), R^{y} is

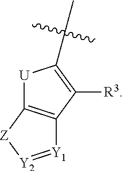

In some embodiments of the compound of Formula (II), R^{y} is

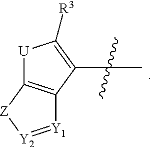

In some embodiments of the compound of Formula (II), R^{y} is

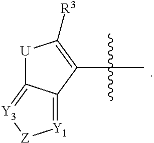

In some embodiments of the compound of Formula (II), R^{y} is

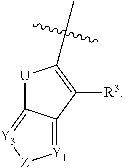

In some embodiments of the compound of Formula (II), each R² is independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂. In some such embodiments, each R² is hydrogen.

In some embodiments of the compound of Formula (II), each R is independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂. In some such embodiments, each R is hydrogen

In some embodiments of the compound of Formula (II), each R³ is independently hydrogen, alkyl, cycloalkyl, alkenyl, cycloalkenyl, alkynyl, aryl, heteroaryl, benzyl, haloalkyl, haloalkenyl, or C(O)R″. In some such embodiments, each R³ is hydrogen

In some embodiments of the compound of Formula (II), R¹ is hydrogen.

In some embodiments of the compound of Formula (II), X₁ and X₂ are O. In other embodiments, X₁ is O and X₂ is S. In other embodiments, X₁ is S and X₂ is O. In other embodiments, X₁ and X₂ are S.

In some embodiments of the compound of Formula (II), n is 0. In other embodiments, n is 1 or 2. In some embodiments, n is 1. In other embodiments, n is 2.

In accordance with a third aspect of the invention, there is provided a pharmaceutical composition comprising a compound according to any of the above aspects of the present invention.

The invention also provides a compound according to any of the above aspects of the present invention for use as a cereblon binder.

The invention also provides a compound or composition according to any of the above aspects of the present invention, for use in medicine.

The invention also provides a compound or composition according to any of the above aspects of the present invention, for use in immune-oncology.

The invention also provides a compound or composition according to any of the above aspects of the present invention, for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

The present invention also provides a method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders; wherein the method comprises administering to a patient in need thereof an effective amount of a compound or composition according to any of the above aspects of the present invention.

In some embodiments of the method, the method further comprises administering at least one additional active agent to the patient. In some embodiments, the at least one additional active agent is an anti-cancer agent or an agent for the treatment of an autoimmune disease. In some embodiments, the at least one additional active agent is a small molecule, a peptide, an antibody, a corticosteroid, or a combination thereof. In some embodiments, the at least one additional active agent is at least one of bortezomib, dexamethasone, and rituximab.

The present invention also provides a combined preparation of a compound of any one of the first to fourth aspects of the present invention and at least one additional active agent, for simultaneous, separate or sequential use in therapy.

In some embodiments of the combined preparation, the at least one additional active agent is an anti-cancer agent or an agent for the treatment of an autoimmune disease. In some embodiments, the at least one additional active agent is a small molecule, a peptide, an antibody, a corticosteroid, or a combination thereof. In some embodiments, the at least one additional active agent is at least one of bortezomib, dexamethasone, and rituximab. In some embodiments, the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

The invention also provides bifunctional compound having the structure:

CLM-L-PTM,

or a pharmaceutically acceptable salt, enantiomer, stereoisomer, solvate, polymorph or prodrug thereof, wherein:
CLM is a cereblon E3 ubiquitin ligase binding moiety;
PTM is a protein targeting moiety; and
L is selected from a bond and a chemical linking moiety covalently coupling the CLM and the PTM; and
wherein the CLM is a compound of any one of claims 1-101, wherein at least one of R, R², R³ and R⁴ contains a group or is modified so as to contain a group through which it can be covalently attached to L or to the PTM.

In some embodiments, L is selected from:

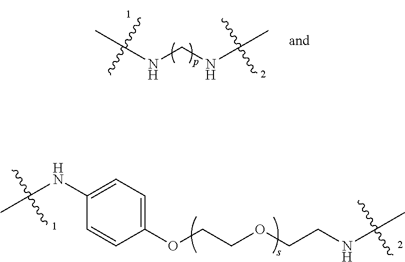

wherein [symbol] indicates attachment to the PTM, and [symbol] indicates attachment to the CLM; p is an integer from 3 to 12; and s is an integer from 1 to 6.

In some embodiments, L is

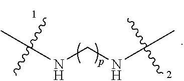

In some embodiments, p is an integer from 4 to 11, from 5 to 10, from 6 to 9, or from 7 to 8.

In some embodiments, L is

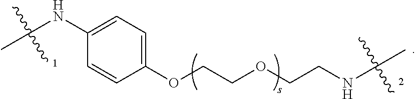

In some embodiments, s is an integer from 2 to 5, or from 3 to 4.

In some embodiments, L is

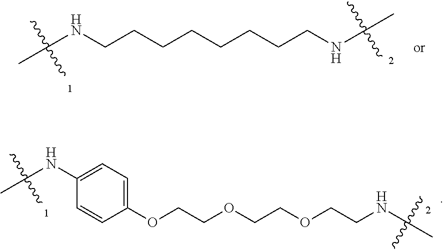 [SMILES: *NCCCCCCCCN* ; *NCCOCCOCCOc1ccc(N*)cc1]

In other embodiments, L is a bond

In some embodiments, the PTM targets BRD4. In some embodiments, the PTM is

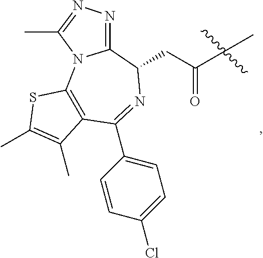 [SMILES: *C(=O)C[C@@H]1N=C(c2ccc(Cl)cc2)c2c(sc(C)c2C)-n2c(C)nnc21]

wherein [symbol] indicates attachment to L.

In some embodiments, at least one of R, R², R³ and R⁴ is modified so as to include a carboxylic acid group or an ester group.

In some embodiments, the bifunctional compound is selected from

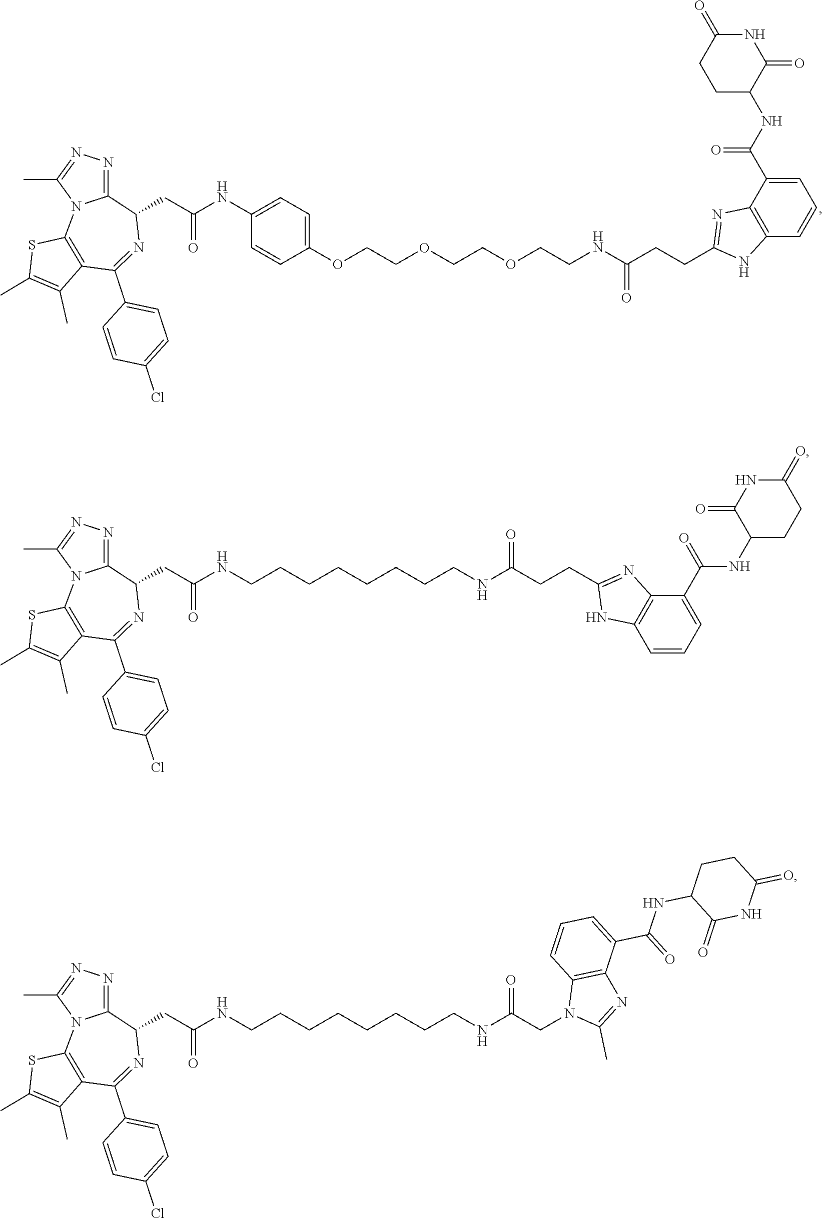 [SMILES: Cc1sc2c(c1C)C(c1ccc(Cl)cc1)=N[C@@H](CC(=O)Nc1ccc(OCCOCCOCCNC(=O)CCc3nc4c(C(=O)NC5CCC(=O)NC5=O)cccc4[nH]3)cc1)c1nnc(C)n1-2 ; Cc1sc2c(c1C)C(c1ccc(Cl)cc1)=N[C@@H](CC(=O)NCCCCCCCCNC(=O)CCc1nc3c(C(=O)NC4CCC(=O)NC4=O)cccc3[nH]1)c1nnc(C)n1-2 ; Cc1sc2c(c1C)C(c1ccc(Cl)cc1)=N[C@@H](CC(=O)NCCCCCCCCNC(=O)Cn1c(C)nc3c(C(=O)NC4CCC(=O)NC4=O)cccc31)c1nnc(C)n1-2]

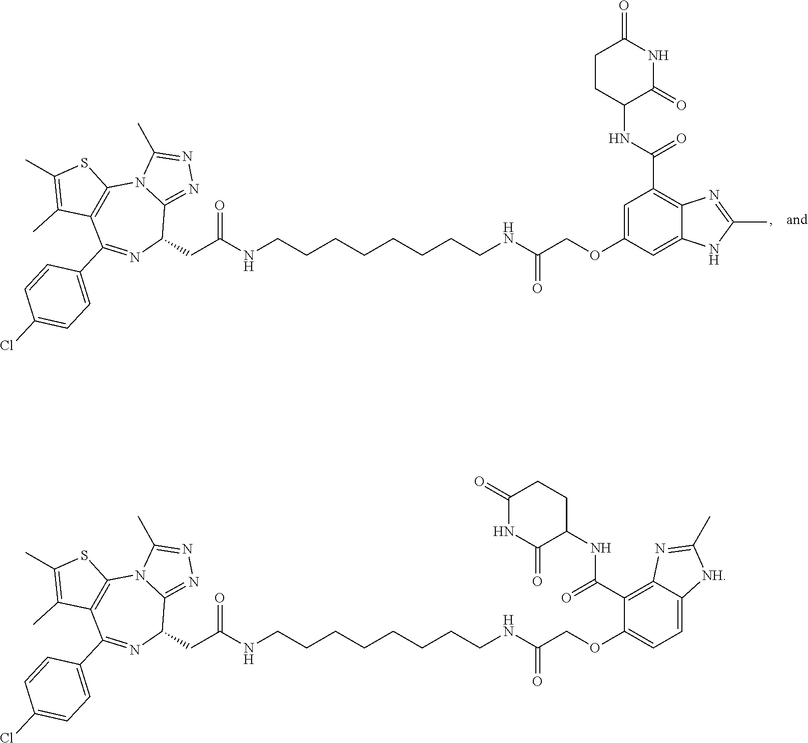 [SMILES: Cc1nc2c(C(=O)NC3CCC(=O)NC3=O)cc(OCC(=O)NCCCCCCCCNC(=O)C[C@@H]3N=C(c4ccc(Cl)cc4)c4c(sc(C)c4C)-n4c(C)nnc43)cc2[nH]1 ; Cc1nc2c(C(=O)NC3CCC(=O)NC3=O)c(OCC(=O)NCCCCCCCCNC(=O)C[C@@H]3N=C(c4ccc(Cl)cc4)c4c(sc(C)c4C)-n4c(C)nnc43)ccc2[nH]1]

As used herein the term “alkyl” is intended to include both unsubstituted alkyl groups, and alkyl groups which are substituted by one or more additional groups—for example —OH, —OR″, —NH₂, —NHR″, —NR″₂, —SO₂R″, —C(O)R″, —CN, or —NO₂. In some embodiments, the alkyl group is an unsubstituted alkyl group. In some embodiments, the alkyl group is a C₁-C₁₂ alkyl, a C₁-C₁₀ alkyl, a C₁-C₅ alkyl, a C₁-C₆ alkyl, or a C₁-C₄ alkyl group.

As used herein the term “alkenyl” is intended to include both unsubstituted alkenyl groups, and alkenyl groups which are substituted by one or more additional groups—for example —OH, —OR″, —NH₂, —NHR″, —NR″₂, —SO₂R″, —C(O)R″, —CN, or —NO₂. In some embodiments, the alkenyl group is an unsubstituted alkenyl group. In some embodiments, the alkenyl group is a C₂-C₁₂ alkenyl, a C₂-C₁₀ alkenyl, a C₂-C₈ alkenyl, a C₂-C₆ alkenyl, or a C₂-C₄ alkenyl group.

As used herein the term “alkynyl” is intended to include both unsubstituted alkynyl groups, and alkynyl groups which are substituted by one or more additional groups—for example —OH, —OR″, halogen, —NH₂, —NHR″, —NR″₂, —SO₂R″, —C(O)R″, —CN, or —NO₂. In some embodiments, the alkynyl group is an unsubstituted alkynyl group. In some embodiments, the alkynyl group is a C₂-C₁₂ alkynyl, a C₂-C₁₀ alkynyl, a C₂-C₈ alkynyl, a C₂-C₆ alkynyl, or a C₂-C₄ alkynyl group.

As used herein the term “aryl” is intended to include both unsubstituted aryl groups, and aryl groups which are substituted by one or more additional groups—for example —OH, —OR″, halogen, —NH₂, —NHR″, —NR″₂, —SO₂R″, —C(O)R″, —CN, or —NO₂. In some embodiments, the aryl group is an unsubstituted aryl group.

In some embodiments, the aryl group is a C₆-C₁₀ aryl, a C₆-C₈ aryl, or a C₆ aryl.

As used herein the term “heteroaryl” is intended to include both unsubstituted heteroaryl groups, and heteroaryl groups which are substituted by one or more additional groups—for example —OH, —OR″, halogen, —NH₂, —NHR″, —NR″₂, —SO₂R″, —C(O)R″, —CN, or —NO₂. In some embodiments, the heteroaryl group is an unsubstituted heteroaryl group. In some embodiments, the heteroaryl group is a C₆-C₁₀ heteroaryl, a C₆-C₈ heteroaryl, a C₆-C₅ heteroaryl, or a C₆ heteroaryl.

As used herein the term “benzyl” is intended to include both unsubstituted benzyl groups, and benzyl groups which are substituted by one or more additional groups—for example —OH, —OR″, halogen, —NH₂, —NHR″, —NR″₂, —SO₂R″, —C(O)R″, —CN, or —NO₂. In some embodiments, the benzyl group is an unsubstituted benzyl group.

### BRIEF DESCRIPTION OF THE FIGURES

FIG. 1 is an assay showing the effect of various compounds of the invention and various reference compounds on SALL4 degradation in the Kelly cell line.

FIG. 2 is an assay showing the effect of various compounds of the invention and various reference compounds on CK1a degradation in the Kelly cell line FIG. 3 is an assay showing the effect of various compounds of the invention and various reference compounds on IKZF1 degradation in the H929 cell line.

FIG. 4 is an assay showing the effect of various compounds of the invention and various reference compounds on IKZF1 degradation in the H929 cell line.

FIG. 5 is an assay showing the effect of various compounds of the invention and various reference compounds on IKZF3 degradation in the H929 cell line

FIG. 6 is an assay showing the effect of various compounds of the invention and various reference compounds on IKZF3 degradation in the H929 cell line

FIG. 7 is an assay showing the effect of various compounds of the invention and various reference compounds on BRD4 degradation in the H929 cell line

FIG. 8 shows the effect of compounds of the invention on formation of ternary complex composed of BRD4-compound-CRBN/DDB1.

FIG. 9 shows the effect of compounds of the invention on formation of ternary complex composed of IKZF1-compound-CRBN/DDB1.

FIG. 10 is a schematic illustration of the general principle for targeted protein degradation upon treatment with a bifunctional compound.

### DETAILED DESCRIPTION OF THE INVENTION

As discussed above, the present invention provides compounds of Formulas (I) and (II), below:

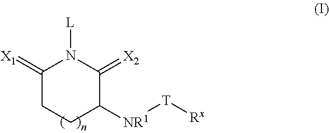

wherein R^{x} is selected from

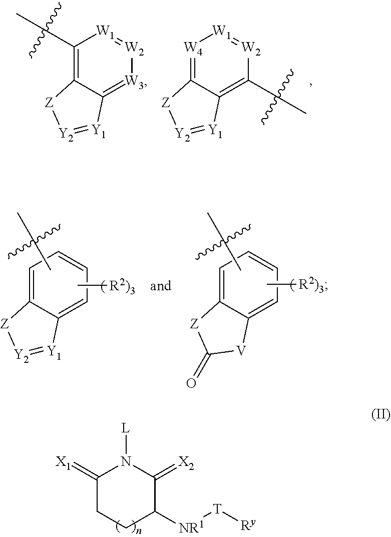

wherein R^{y} is selected from

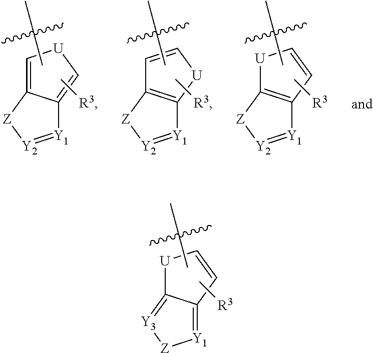

wherein L, X₁, X₂, Y₁, Y₂, Y₃, W₁, W₂, W₃, W₄, R¹, R², T, U, V and Z are as defined above. Binding of the above compounds to cereblon may alter the specificity of the CRL4^{CRBN} complexes, and induce association of novel substrate proteins, followed by their ubiquitination and degradation. Examples of such proteins include, but are not limited to, IKZF1 and IKZF3.

The above compounds may modulate cereblon in a unique way allowing CRL4^{CRBN} ubiquitin ligase complex to recognise different substrates to those which it would otherwise recognise, and target them for degradation. Consequently, the compounds of the present invention are expected to broaden/modify CRBN's antiproliferative activity, thus extending the range of cancer types sensitive to treatment with CMAs.

The compounds of the present invention are advantageous in terms of their synthetic feasibility. The synthesis of the compounds can be summarized as follows:

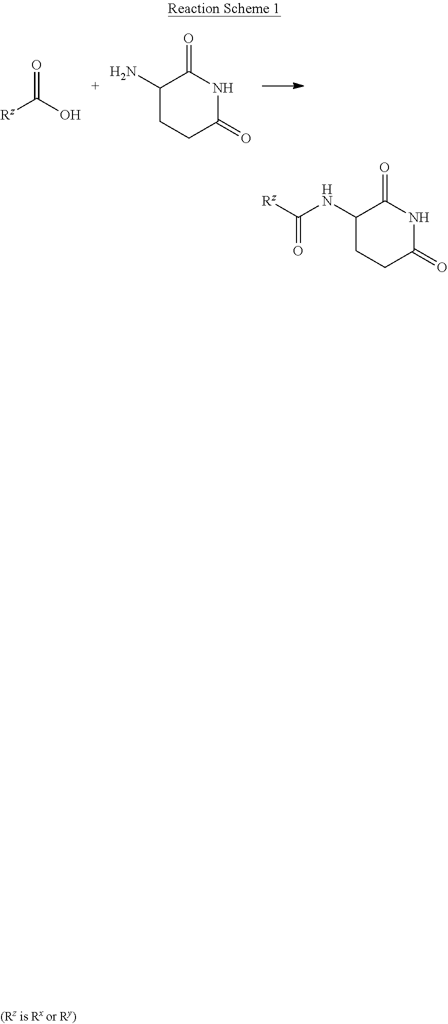

Example compounds of the present invention are shown below:

| Compound | |
|---|---|
| number | Structure |
| 1 | 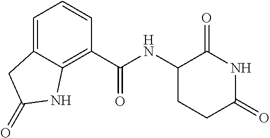 [SMILES: O=C1CCC(NC(=O)c2cccc3c2NC(=O)C3)C(=O)N1] |
| 2 | 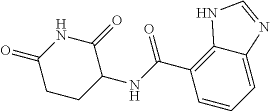 [SMILES: O=C1CCC(NC(=O)c2cccc3nc[nH]c23)C(=O)N1] |
| 3 | 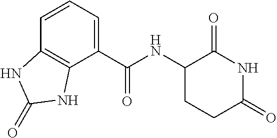 [SMILES: O=C1CCC(NC(=O)c2cccc3[nH]c(=O)[nH]c23)C(=O)N1] |
| 4 | 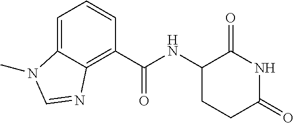 [SMILES: Cn1cnc2c(C(=O)NC3CCC(=O)NC3=O)cccc21] |
| 5 | 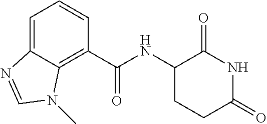 [SMILES: Cn1cnc2cccc(C(=O)NC3CCC(=O)NC3=O)c21] |
| 6 | 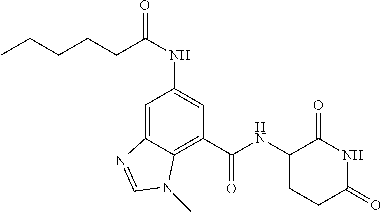 [SMILES: CCCCCC(=O)Nc1cc(C(=O)NC2CCC(=O)NC2=O)c2c(c1)ncn2C] |
| 7 | 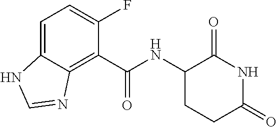 [SMILES: O=C1CCC(NC(=O)c2c(F)ccc3[nH]cnc23)C(=O)N1] |
| 8 | 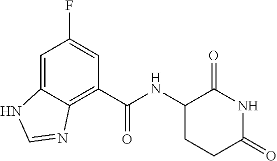 [SMILES: O=C1CCC(NC(=O)c2cc(F)cc3[nH]cnc23)C(=O)N1] |
| 9 | 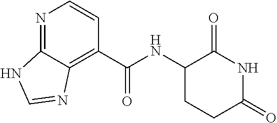 [SMILES: O=C1CCC(NC(=O)c2ccnc3[nH]cnc23)C(=O)N1] |
| 10 | 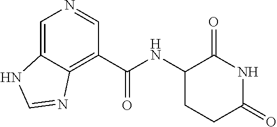 [SMILES: O=C1CCC(NC(=O)c2cncc3[nH]cnc23)C(=O)N1] |
| 11 | 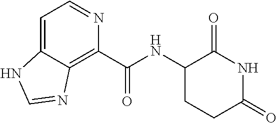 [SMILES: O=C1CCC(NC(=O)c2nccc3[nH]cnc23)C(=O)N1] |
| 12 | 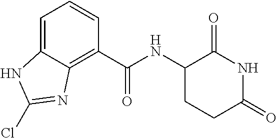 [SMILES: O=C1CCC(NC(=O)c2cccc3[nH]c(Cl)nc23)C(=O)N1] |
| 14 | 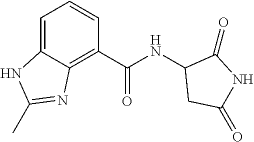 [SMILES: Cc1nc2c(C(=O)NC3CC(=O)NC3=O)cccc2[nH]1] |
| 15 | 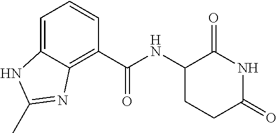 [SMILES: Cc1nc2c(C(=O)NC3CCC(=O)NC3=O)cccc2[nH]1] |
| 17 | 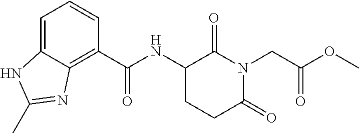 |
| 19 | 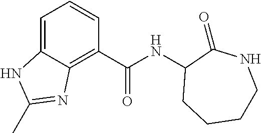 [SMILES: Cc1nc2c(C(=O)NC3CCCCNC3=O)cccc2[nH]1] |
| 20 | 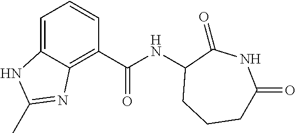 [SMILES: Cc1nc2c(C(=O)NC3CCCC(=O)NC3=O)cccc2[nH]1] |
| 22 | 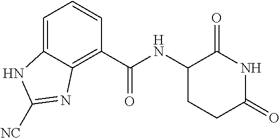 [SMILES: N#Cc1nc2c(C(=O)NC3CCC(=O)NC3=O)cccc2[nH]1] |
| 23 | 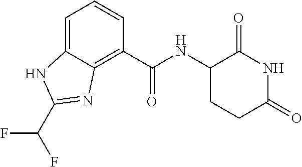 [SMILES: O=C1CCC(NC(=O)c2cccc3[nH]c(C(F)F)nc23)C(=O)N1] |
| 24 | 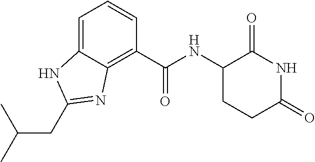 [SMILES: CC(C)Cc1nc2c(C(=O)NC3CCC(=O)NC3=O)cccc2[nH]1] |
| 25 | 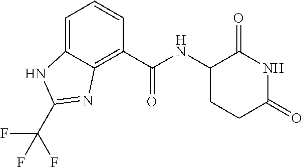 [SMILES: O=C1CCC(NC(=O)c2cccc3[nH]c(C(F)(F)F)nc23)C(=O)N1] |
| 26 | 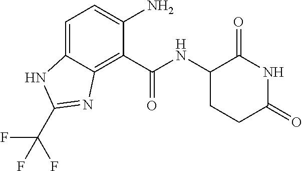 [SMILES: Nc1ccc2[nH]c(C(F)(F)F)nc2c1C(=O)NC1CCC(=O)NC1=O] |
| 27 | 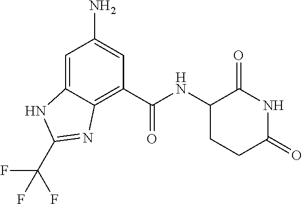 [SMILES: Nc1cc(C(=O)NC2CCC(=O)NC2=O)c2nc(C(F)(F)F)[nH]c2c1] |
| 28 | 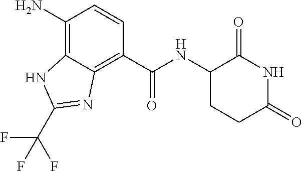 [SMILES: Nc1ccc(C(=O)NC2CCC(=O)NC2=O)c2nc(C(F)(F)F)[nH]c12] |
| 29 | 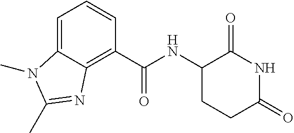 [SMILES: Cc1nc2c(C(=O)NC3CCC(=O)NC3=O)cccc2n1C] |
| 30 | 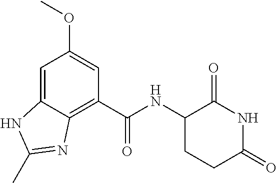 [SMILES: COc1cc(C(=O)NC2CCC(=O)NC2=O)c2nc(C)[nH]c2c1] |
| 31 | 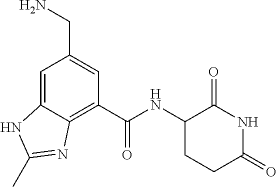 [SMILES: Cc1nc2c(C(=O)NC3CCC(=O)NC3=O)cc(CN)cc2[nH]1] |
| 32 | 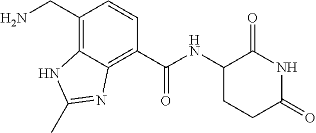 [SMILES: Cc1nc2c(C(=O)NC3CCC(=O)NC3=O)ccc(CN)c2[nH]1] |
| 33 | 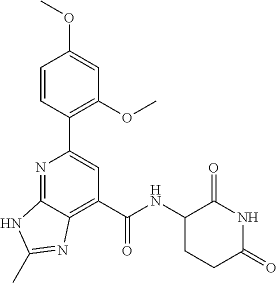 [SMILES: COc1ccc(-c2cc(C(=O)NC3CCC(=O)NC3=O)c3nc(C)[nH]c3n2)c(OC)c1] |
| 35 | 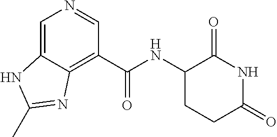 [SMILES: Cc1nc2c(C(=O)NC3CCC(=O)NC3=O)cncc2[nH]1] |
| 36 | 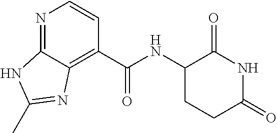 [SMILES: Cc1nc2c(C(=O)NC3CCC(=O)NC3=O)ccnc2[nH]1] |
| 37 | 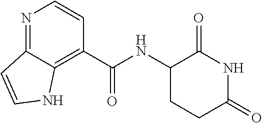 [SMILES: O=C1CCC(NC(=O)c2ccnc3cc[nH]c23)C(=O)N1] |
| 38 | 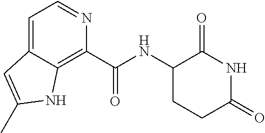 [SMILES: Cc1cc2ccnc(C(=O)NC3CCC(=O)NC3=O)c2[nH]1] |
| 39 | 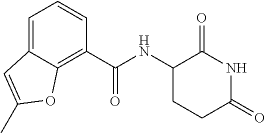 [SMILES: Cc1cc2cccc(C(=O)NC3CCC(=O)NC3=O)c2o1] |
| 40 | 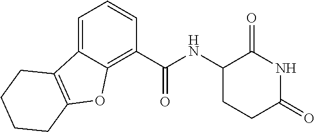 [SMILES: O=C1CCC(NC(=O)c2cccc3c4c(oc23)CCCC4)C(=O)N1] |
| 41 | 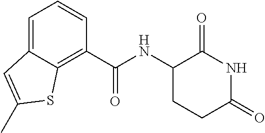 [SMILES: Cc1cc2cccc(C(=O)NC3CCC(=O)NC3=O)c2s1] |
| 42 | 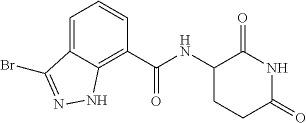 [SMILES: O=C1CCC(NC(=O)c2cccc3c(Br)n[nH]c23)C(=O)N1] |
| 43 | 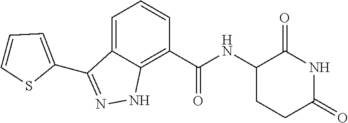 [SMILES: O=C1CCC(NC(=O)c2cccc3c(-c4cccs4)n[nH]c23)C(=O)N1] |
| 45 | 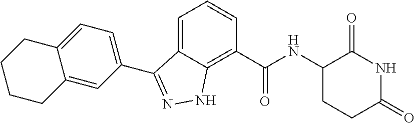 [SMILES: O=C1CCC(NC(=O)c2cccc3c(-c4ccc5c(c4)CCCC5)n[nH]c23)C(=O)N1] |
| 46 | 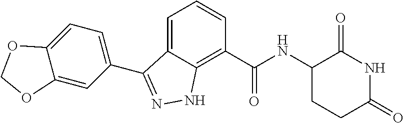 [SMILES: O=C1CCC(NC(=O)c2cccc3c(-c4ccc5c(c4)OCO5)n[nH]c23)C(=O)N1] |
| 47 | 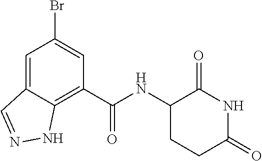 [SMILES: O=C1CCC(NC(=O)c2cc(Br)cc3cn[nH]c23)C(=O)N1] |
| 48 | 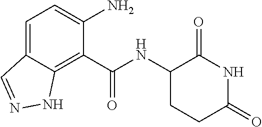 [SMILES: Nc1ccc2cn[nH]c2c1C(=O)NC1CCC(=O)NC1=O] |
| 50 | 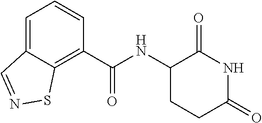 [SMILES: O=C1CCC(NC(=O)c2cccc3cnsc23)C(=O)N1] |
| 51 | 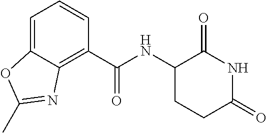 [SMILES: Cc1nc2c(C(=O)NC3CCC(=O)NC3=O)cccc2o1] |
| 52 | 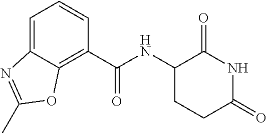 [SMILES: Cc1nc2cccc(C(=O)NC3CCC(=O)NC3=O)c2o1] |
| 53 | 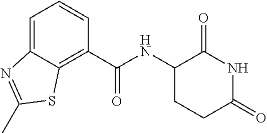 [SMILES: Cc1nc2cccc(C(=O)NC3CCC(=O)NC3=O)c2s1] |
| 54 | 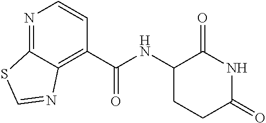 [SMILES: O=C1CCC(NC(=O)c2ccnc3scnc23)C(=O)N1] |
| 55 | 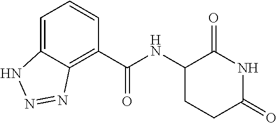 [SMILES: O=C1CCC(NC(=O)c2cccc3[nH]nnc23)C(=O)N1] |
| 57 | 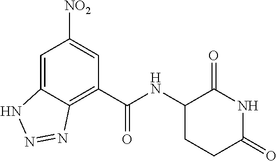 [SMILES: O=C1CCC(NC(=O)c2cc([N+](=O)[O-])cc3[nH]nnc23)C(=O)N1] |
| 58 | 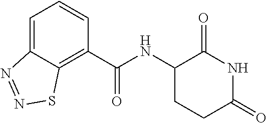 [SMILES: O=C1CCC(NC(=O)c2cccc3nnsc23)C(=O)N1] |
| 58 | 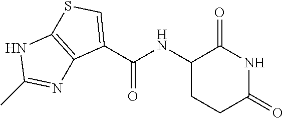 [SMILES: Cc1nc2c(C(=O)NC3CCC(=O)NC3=O)csc2[nH]1] |
| 60 | 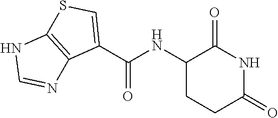 [SMILES: O=C1CCC(NC(=O)c2csc3[nH]cnc23)C(=O)N1] |
| 61 | 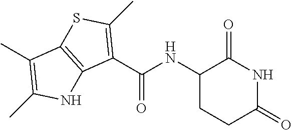 [SMILES: Cc1[nH]c2c(C(=O)NC3CCC(=O)NC3=O)c(C)sc2c1C] |
| 62 | 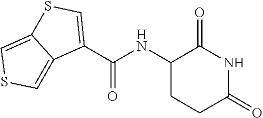 [SMILES: O=C1CCC(NC(=O)c2csc3cscc23)C(=O)N1] |
| 63 | 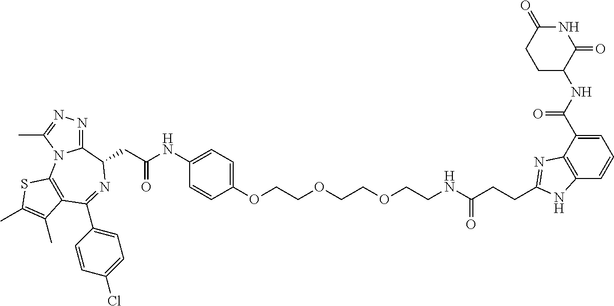 [SMILES: Cc1sc2c(c1C)C(c1ccc(Cl)cc1)=N[C@@H](CC(=O)Nc1ccc(OCCOCCOCCNC(=O)CCc3nc4c(C(=O)NC5CCC(=O)NC5=O)cccc4[nH]3)cc1)c1nnc(C)n1-2] |
| 64 | 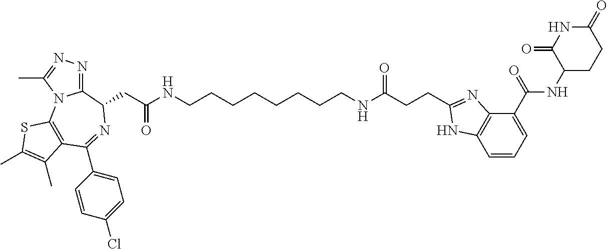 [SMILES: Cc1sc2c(c1C)C(c1ccc(Cl)cc1)=N[C@@H](CC(=O)NCCCCCCCCNC(=O)CCc1nc3c(C(=O)NC4CCC(=O)NC4=O)cccc3[nH]1)c1nnc(C)n1-2] |
| 65 | 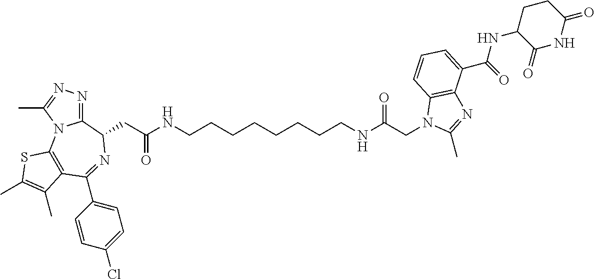 [SMILES: Cc1sc2c(c1C)C(c1ccc(Cl)cc1)=N[C@@H](CC(=O)NCCCCCCCCNC(=O)Cn1c(C)nc3c(C(=O)NC4CCC(=O)NC4=O)cccc31)c1nnc(C)n1-2] |
| 66 | 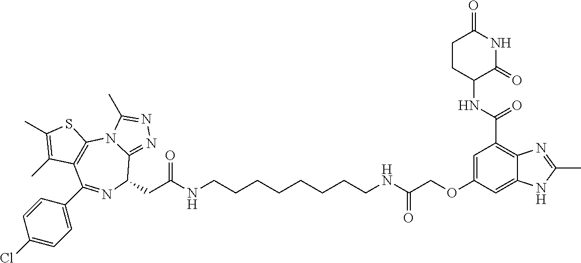 [SMILES: Cc1nc2c(C(=O)NC3CCC(=O)NC3=O)cc(OCC(=O)NCCCCCCCCNC(=O)C[C@@H]3N=C(c4ccc(Cl)cc4)c4c(sc(C)c4C)-n4c(C)nnc43)cc2[nH]1] |
| 67 | 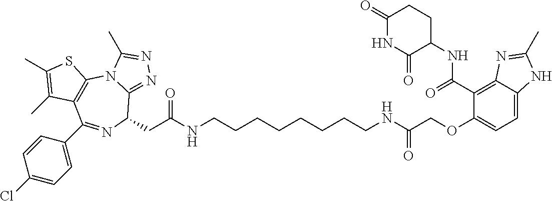 [SMILES: Cc1nc2c(C(=O)NC3CCC(=O)NC3=O)c(OCC(=O)NCCCCCCCCNC(=O)C[C@@H]3N=C(c4ccc(Cl)cc4)c4c(sc(C)c4C)-n4c(C)nnc43)ccc2[nH]1] |

As discussed in the Examples section, the present inventors have found that the above compounds exhibit similar cereblon binding capabilities to that of the known CMA, CC-122. Despite the pharmaceutical activity of the known CMAs such as CC-122, patients often develop resistance to these compounds. The use of novel compounds—such as those of the present invention, as described above—may help to overcome this clinical obstacle.

One of the serious disadvantages of the currently available CMAs is their safety profile. For example, the teratogenicity of the CMAs is dependent upon the extent to which the CMAs induce degradation of SALL4 transcription factor. Known CMAs induce degradation of several proteins (including SALL4) which bind to CRL4^{CRBN} ligase only in presence of the CMA. SALL4 degradation, observed under treatment with CMAs, is responsible (at least partly) for the teratogenicity of the CMAs. Compounds with diminished capability to induce SALL4 degradation may demonstrate an improved safety profile.

The compounds of the present invention may also possess pharmaceutically advantageous properties, such as increased stability and improved ADMET (absorption, distribution, metabolism, excretion, and/or toxicity) properties.

The compounds of the present invention may be useful in the treatment of various diseases and disorders, including (but not limited to):
1) Cancer. The compounds provided herein can be used for treating, preventing or managing either primary or metastatic tumors. Specific examples of cancer include, but are not limited to, cancers of the skin, such as melanoma; lymph node; breast; cervix; uterus; gastrointestinal tract; lung; ovary; prostate; colon; rectum; mouth; brain; head and neck; throat; testes; kidney; pancreas; bone; spleen; liver; bladder; larynx; nasal passages, and AIDS-related cancers and hematological malignancies.
   a) Hematological malignancies include leukemia, lymphoma, multiple myeloma or smoldering myeloma.
      Leukemia can be selected from: acute leukemia, acute lymphoblastic leukemia (ALL), chronic lymphocytic leukemia (CLL), acute myelogenous leukemia, acute myeloid leukemia (AML), adult acute basophilic leukemia, adult acute eosinophilic leukemia, adult acute megakaryoblastic leukemia, adult acute minimally differentiated myeloid leukemia, adult acute monoblastic leukemia, adult acute monocytic leukemia, adult acute myeloblastic leukemia with maturation, adult acute myeloblastic leukemia without maturation, adult acute myeloid leukemia with abnormalities, adult acute myelomonocytic leukemia, adult erythroleukemia, adult pure erythroid leukemia, secondary acute myeloid leukemia, untreated adult acute myeloid leukemia, adult acute myeloid leukemia in remission, adult acute promyelocytic leukemia with PML-RARA, alkylating agent-related acute myeloid leukemia, prolymphocytic leukemia, and chronic myelomonocytic leukemia, refractory hairy cell leukemia, T-cell large granular lymphocyte leukemia, relapsed or refractory chronic lymphocytic leukemia.
      Lymphoma can be selected from the group consisting of: adult grade III lymphomatoid granulomatosis, adult nasal type extranodal NK/T-cell lymphoma, anaplastic large cell lymphoma, angioimmunoblastic T-cell lymphoma, cutaneous B-Cell non-Hodgkin lymphoma, extranodal marginal zone lymphoma of mucosa-associated lymphoid tissue, hepatosplenic T-cell lymphoma, intraocular lymphoma, lymphomatous involvement of non-cutaneous extranodal site, mature T-cell and K-cell non-Hodgkin lymphoma, nodal marginal zone lymphoma, post-transplant lymphoproliferative disorder, recurrent adult Burkitt lymphoma, recurrent adult diffuse large cell lymphoma, recurrent adult diffuse mixed cell lymphoma, recurrent adult diffuse small cleaved cell lymphoma, recurrent adult grade III lymphomatoid granulomatosis, recurrent adult immunoblastic lymphoma, recurrent adult lymphoblastic lymphoma, recurrent adult T-cell leukemia/lymphoma, recurrent cutaneous T-cell non-Hodgkin lymphoma, recurrent grade 1 follicular lymphoma, recurrent grade 2 follicular lymphoma, recurrent grade 3 follicular lymphoma, recurrent mantle cell lymphoma, recurrent marginal zone lymphoma, recurrent mycosis fungoides and Sezary syndrome, recurrent small lymphocytic lymphoma, Richter syndrome, small intestinal lymphoma, splenic marginal zone lymphoma, testicular lymphoma, Waldenstrom macroglobulinemia, adult T-cell leukemia-lymphoma, peripheral T-cell lymphoma, B-cell lymphoma, Hodgkin's disease, cutaneous T-cell lymphoma, diffuse large B-cell lymphoma, MALT lymphoma, mantle cell lymphoma, non-Hodgkins lymphoma, central nervous system lymphoma, refractory primary-cutaneous large B-cell lymphoma (Leg-type), refractory anemia, refractory anemia with excess blasts, refractory anemia with ringed sideroblasts, refractory cytopenia with multilineage dysplasia, secondary myelodysplastic syndromes, myelodysplastic syndrome, and myeloproliferative disease.
2) Autoimmune diseases, such as: Acute disseminated encephalomyelitis, acute motor axonal neuropathy, Addison's disease, adiposis dolorosa, adult-onset Still's disease, alopecia areata, ankylosing spondylitis, anti-glomerular basement membrane nephritis, anti-neutrophil cytoplasmic antibody-associated vasculitis, anti-N-methyl-D-aspartate receptor encephalitis, antiphospholipid syndrome, antisynthetase syndrome, aplastic anemia, autoimmune angioedema, autoimmune encephalitis, autoimmune enteropathy, autoimmune hemolytic anemia, autoimmune hepatitis, autoimmune inner ear disease, autoimmune lymphoproliferative syndrome, autoimmune neutropenia, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy, autoimmune thrombocytopenic purpura, autoimmune thyroiditis, autoimmune urticaria, autoimmune uveitis, balo concentric sclerosis, Behget's disease, Bickerstaff's encephalitis, bullous pemphigoid, celiac disease, chronic fatigue syndrome, chronic inflammatory demyelinating polyneuropathy, churg-Strauss syndrome, cicatricial pemphigoid, cogan syndrome, cold agglutinin disease, complex regional pain syndrome, CREST syndrome, Crohn's disease, dermatitis herpetiformis, dermatomyositis, diabetes mellitus type 1, discoid lupus erythematosus, endometriosis, enthesitis, enthesitis-related arthritis, eosinophilic esophagitis, eosinophilic fasciitis, epidermolysis bullosa acquisita, erythema nodosum. essential mixed cryoglobulinemia, evans syndrome, felty syndrome, fibromyalgia, gastritis, gestational pemphigoid, giant cell arteritis, goodpasture syndrome, Graves' disease, graves ophthalmopathy, Guillain-Barre syndrome, hashimoto's encephalopathy, hashimoto thyroiditis, Henoch-Schonlein purpura, hidradenitis suppurativa, idiopathic inflammatory demyelinating diseases, igG4-related systemic disease, inclusion body myositis, inflamatory bowel disease (IBD), intermediate uveitis, interstitial cystitis, juvenile arthritis, kawasaki's disease, Lambert-Eaton myasthenic syndrome, leukocytoclastic vasculitis, Lichen planus, Lichen sclerosus, ligneous conjunctivitis, linear IgA disease, lupus nephritis, lupus vasculitis, lyme disease (Chronic), Meniere's disease, microscopic colitis, microscopic polyangiitis, mixed connective tissue disease, Mooren's ulcer, morphea, Mucha-Habermann disease, multiple sclerosis, myasthenia gravis, myocarditis, myositis, neuromyelitis optica, neuromyotonia, opsoclonus myoclonus syndrome, optic neuritis, Ord's thyroiditis, palindromic rheumatism, paraneoplastic cerebellar degeneration, Parry Romberg syndrome, Parsonage-Turner syndrome, pediatric autoimmune neuropsychiatric disorder associated with streptococcus, Pemphigus vulgaris, pernicious anemia, Pityriasis lichenoides et varioliformis acuta, POEMS syndrome, polyarteritis nodosa, polymyalgia rheumatica, polymyositis, postmyocardial infarction syndrome, postpericardiotomy syndrome, primary biliary cirrhosis, primary immunodeficiency, primary sclerosing cholangitis, progressive inflammatory neuropathy, psoriasis, psoriatic arthritis, pure red cell aplasia, pyoderma gangrenosum, Raynaud's phenomenon, reactive arthritis, relapsing polychondritis, restless leg syndrome, retroperitoneal fibrosis, rheumatic fever, rheumatoid arthritis, rheumatoid vasculitis, sarcoidosis, Schnitzler syndrome, scleroderma, Sjogren's syndrome, stiff person syndrome, subacute bacterial endocarditis, Susac's syndrome, Sydenham chorea, sympathetic ophthalmia, systemic lupus erythematosus, systemic scleroderma, thrombocytopenia, Tolosa-Hunt syndrome, transverse myelitis, ulcerative colitis, undifferentiated connective tissue disease, urticaria, urticarial vasculitis, vasculitis and vitiligo;
3) Diseases and disorders associated with, or characterized by, undesired angiogenesis, including inflammatory diseases, autoimmune diseases, pain, viral diseases, genetic diseases, allergic diseases, bacterial diseases, ocular neovascular diseases, choroidal neovascular diseases, retina neovascular diseases, and rubeosis (neovascularization of the angle). Specific examples of the diseases and disorders associated with, or characterized by, undesired angiogenesis include, but are not limited to: arthritis, endometriosis, Crohn's disease, heart failure, advanced heart failure, renal impairment, endotoxemia, toxic shock syndrome, osteoarthritis, retrovirus replication, wasting, meningitis, silica-induced fibrosis, asbestos-induced fibrosis, veterinary disorder, malignancy-associated hypercalcemia, stroke, circulatory shock, periodontitis, gingivitis, macrocytic anemia, refractory anemia, and 5q-deletion syndrome, nociceptive pain, neuropathic pain, mixed pain of nociceptive and neuropathic pain, visceral pain, migraine, headache and postoperative pain. Examples of nociceptive pain include, but are not limited to, pain associated with chemical or thermal burns, cuts of the skin, contusions of the skin, osteoarthritis, rheumatoid arthritis, tendonitis, and myofascial pain. Examples of neuropathic pain include, but are not limited to, CRPS type 1, CRPS type 11, reflex sympathetic dystrophy (RSD), reflex neurovascular dystrophy, reflex dystrophy, sympathetically maintained pain syndrome, causalgia, Sudeck atrophy of bone, algoneurodystrophy, shoulder hand syndrome, post-traumatic dystrophy, trigeminal neuralgia, post herpetic neuralgia, cancer related pain, phantom limb pain, fibromyalgia, chronic fatigue syndrome, spinal cord injury pain, central post-stroke pain, radiculopathy, diabetic neuropathy, post-stroke pain, luetic neuropathy, and other painful neuropathic conditions such as those induced by drugs such as vincristine and velcade;
4) Macular Degeneration (“MD”) and related syndromes, such as: atrophic (dry) MD, exudative (wet) MD, age-related maculopathy (ARM), choroidal neovascularisation (CNVM), retinal pigment epithelium detachment (PED), and atrophy of retinal pigment epithelium (RPE);
5) Skin diseases such as: keratoses and related symptoms, skin diseases or disorders characterized with overgrowths of the epidermis, acne, and wrinkles. Examples of skin diseases or disorders characterized with overgrowths of the epidermis include, but are not limited to, any conditions, diseases or disorders marked by the presence of overgrowths of the epidermis, including but not limited to, infections associated with papilloma virus, arsenical keratoses, sign of Leser-Trelat, warty dyskeratoma (WD), trichostasis spinulosa (TS), erythrokeratodermia variabilis (EKV), ichthyosis fetalis (harlequin ichthyosis), knuckle pads, cutaneous melanoacanthoma, porokeratosis, psoriasis, squamous cell carcinoma, confluent and reticulated papillomatosis (CRP), acrochordons, cutaneous horn, cowden disease (multiple hamartoma syndrome), dermatosis papulosa nigra (DPN), epidermal nevus syndrome (ENS), ichthyosis vulgaris, molluscum contagiosum, prurigo nodularis, and acanthosis nigricans (AN);
6) Pulmonary disorders, such as pulmonary hypertension and related disorders. Examples of pulmonary hypertension and related disorders include, but are not limited to: primary pulmonary hypertension (PPH); secondary pulmonary hypertension (SPH); familial PPH; sporadic PPH; precapillary pulmonary hypertension; pulmonary arterial hypertension (PAH); pulmonary artery hypertension; idiopathic pulmonary hypertension; thrombotic pulmonary arteriopathy (TPA); plexogenic pulmonary arteriopathy; functional classes I to IV pulmonary hypertension; and pulmonary hypertension associated with, related to, or secondary to, left ventricular dysfunction, mitral valvular disease, constrictive pericarditis, aortic stenosis, cardiomyopathy, mediastinal fibrosis, anomalous pulmonary venous drainage, pulmonary venoocclusive disease, collagen vasular disease, congenital heart disease, HIV virus infection, drugs and toxins such as fenfluramines, congenital heart disease, pulmonary venous hypertension, chronic obstructive pulmonary disease, interstitial lung disease, sleep-disordered breathing, alveolar hypoventilation disorder, chronic exposure to high altitude, neonatal lung disease, alveolar-capillary dysplasia, sickle cell disease, other coagulation disorder, chronic thromboemboli, connective tissue disease, lupus including systemic and cutaneous lupus, schistosomiasis, sarcoidosis or pulmonary capillary hemangiomatosis;
7) Asbestos-related disorders, such as: mesothelioma, asbestosis, malignant pleural effusion, benign exudative effusion, pleural plaques, pleural calcification, diffuse pleural thickening, rounded atelectasis, fibrotic masses, and lung cancer;
8) Parasitic diseases and disorders caused by human intracellular parasites such as, but not limited to, P. falcifarium, P. ovale, P. vivax, P. malariae, L. donovari, L. infanium, L. aethiopica, L. major, L. tropica, L mexicana, L braziliensis, T. Gondii, B. microti, B. divergens, B. coli, C. parvum, C. cayetanensis, E. histolytica, I. belli, S. monsonii, S. haemolobium, Trypanosoma ssp., Toxoplasma ssp., and O. volvulus. Other diseases and disorders caused by non-human intracellular parasites such as, but not limited to, Babesia bovis, Babesia canis, Banesia Gibsoni, Besnoitia darlingi, Cytauxzoon felis, Eimeria ssp., Hammondia ssp., and Theileria ssp., are also encompassed. Specific examples include, but are not limited to, malaria, babesiosis, trypanosomiasis, leishmaniasis, toxoplasmosis, meningoencephalitis, keratitis, amebiasis, giardiasis, cryptosporidiosis, isosporiasis, cyclosporiasis, microsporidiosis, ascariasis, trichuriasis, ancylostomiasis, strongyloidiasis, toxocariasis, trichinosis, lymphatic filariasis, onchocerciasis, filariasis, schistosomiasis, and dermatitis caused by animal schistosomes;
9) Immunodeficiency disorders, which include, but are not limited to, adenosine deaminase deficiency, antibody deficiency with normal or elevated Igs, ataxia-tenlangiectasia, bare lymphocyte syndrome, common variable immunodeficiency, Ig deficiency with hyper-IgM, Ig heavy chain deletions, IgA deficiency, immunodeficiency with thymoma, reticular dysgenesis, Nezelof syndrome, selective IgG subclass deficiency, transient hypogammaglobulinemia of infancy, Wistcott-Aldrich syndrome, X-linked agammaglobulinemia, X-linked severe combined immunodeficiency;
10) Atherosclerosis and related conditions, such as: all forms of conditions involving atherosclerosis, including restenosis after vascular intervention such as angioplasty, stenting, atherectomy and grafting;
11) Hemoglobinopathy and related disorders, such as sickle cell anemia, and any other disorders related to the differentiation of CD34+ cells;
12) TNFα related disorders, such as: endotoxemia or toxic shock syndrome; cachexia; adult respiratory distress syndrome; bone resorption diseases such as arthritis; hypercalcemia; Graft versus Host Reaction; cerebral malaria; inflammation; tumor growth; chronic pulmonary inflammatory diseases; reperfusion injury; myocardial infarction; stroke; circulatory shock; rheumatoid arthritis; Crohn's disease; HIV infection and AIDS; other disorders such as rheumatoid arthritis, rheumatoid. spondylitis, osteoarthritis, psoriatic arthritis and other arthritic conditions, septic shock, septis, endotoxic shock, graft versus host disease, wasting, Crohn's disease, ulcerative colitis, multiple sclerosis, systemic lupus erythromatosis, ENL in leprosy, HIV, AIDS, and opportunistic infections in AIDS; disorders such as septic shock, sepsis, endotoxic shock, hemodynamic shock and sepsis syndrome, post ischemic reperfusion injury, malaria, mycobacterial infection, meningitis, psoriasis, congestive heart failure, fibrotic disease, cachexia, graft rejection, oncogenic or cancerous conditions, asthma, autoimmune disease, radiation damages, and hyperoxic alveolar injury; viral infections, such as those caused by the herpes viruses; viral conjunctivitis; or atopic dermatitis.

The compounds of the present invention may also be useful in preventing, treating, or reducing the risk of developing graft versus host disease (GVHD) or transplant rejection.

The compounds of the present invention may also inhibit the production of certain cytokines including, but not limited to, TNF-α, IL-1β, IL-12, IL-18, GM-CSF, IL-10, TGF-β and/or IL-6. The present compounds may stimulate the production of certain cytokines, and also act as a costimulatory signal for T cell activation, resulting in increased production of cytokines such as, but not limited to, IL-12, IL-2, IL-10, TGF-β and/or IFN-γ. In addition, compounds provided herein can enhance the effects of NK cells and antibody-mediated cellular cytotoxicity (ADCC). Further, compounds provided herein may be immunomodulatory and/or cytotoxic, and thus may be useful as chemotherapeutic agents.

### EXAMPLES

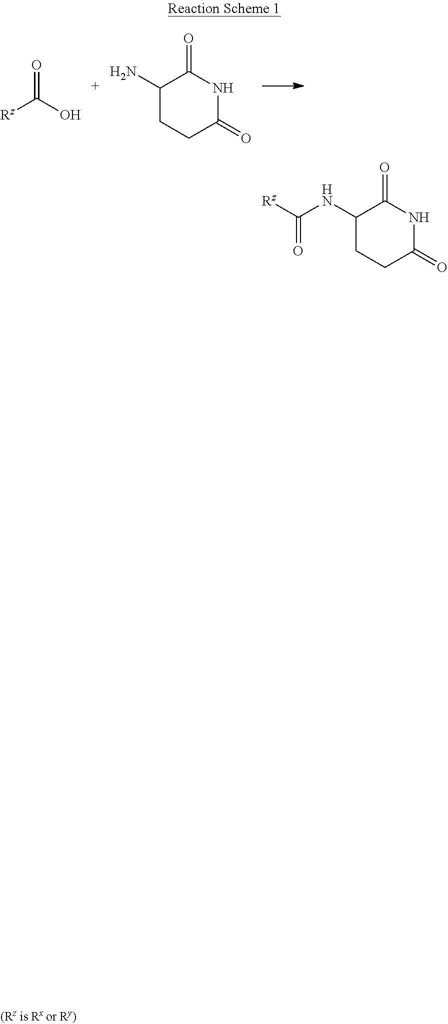

### Synthetic Conditions A

An appropriate acid (R^{z}COOH in the above Reaction Scheme 1) (1. eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.). After removal of the solvent under reduced pressure, the crude product was purified by preparative HPLC, flash column chromatography or preparative TLC.

### Synthetic Conditions B

DIPEA (2-3 eq) was added to a solution of an appropriate acid (R^{z}COOH in the above Reaction Scheme 1), DMAP (0-0.1 eq), HATU (1.0-1.5 eq) and 3-aminopiperidine-2,6-dione hydrochloride (1.2-3.0 eq) in DMF (0.1-0.5 M). The reaction mixture was stirred overnight at room temperature (20-25° C.). After removal of the solvent under reduced pressure, the crude product was purified by preparative HPLC, flash column chromatography or preparative TLC.

### Synthetic Conditions C

CDI (1.2-2 eq) was added to a solution of an appropriate acid (R^{z}COOH in the above Reaction Scheme 1) in DMF (0.1-0.5 M) and stirred for 1 h at 50° C. After cooling to room temperature, 3-aminopiperidine-2,6-dione hydrochloride (1.2-1.5 equiv) was added and the reaction mixture was stirred overnight at room temperature (20-25° C.). After removal of the solvent under reduced pressure, the crude product was purified by preparative HPLC, flash column chromatography or preparative TLC.

### Example 1: Synthesis of N-(2,6-dioxopiperidin-3-yl)-2-oxoindoline-7-carboxamide (1)

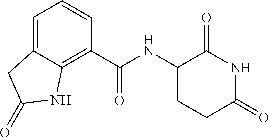 [SMILES: O=C1CCC(NC(=O)c2cccc3c2NC(=O)C3)C(=O)N1]

This compound was synthesized using the general procedure shown in Reaction Scheme 1 and Synthetic Conditions A, above (32% yield), and 2-oxoindoline-7-carboxylic acid (20 mg) as a starting material.

¹H NMR: (500 MHz, DMSO) δ 10.91 (s, 1H), 9.82 (s, 1H), 8.83 (d, J=8.1 Hz, 1H) 7.72-7.64 (m, 1H), 7.42-7.36 (m, 1H), 7.09-7.02 (m, 1H), 4.86-4.75 (m, 1H), 3.55 (s, 2H), 2.88-2.74 (m, 1H), 2.62-2.53 (m, 1H), 2.22-2.08 (m, 1H), 2.04-1.97 (m, 1H)

LCMS (m/z [M+H]⁺): 287.8

### Example 2: Synthesis of N-(2,6-dioxopiperidin-3-yl)-1H-1,3-benzodiazole-7-carboxamide (2)

To a solution of 3-aminopiperidine-2,6-dione (0.96 g, 7.5 mmol) and N-hydroxybenzotriazole (1.22 g, 9.0 mmol) in DMF (15 mL) were added 1H-benzo[d]imidazole-7-carboxylic acid (8.25 g, 1.3 mmol), DMAP (37 mg, 0.30 mmol), and EDC (1.40 g, 9.0 mmol). The reaction mixture was stirred overnight at room temperature. Water (30 mL) was added and the obtained solution was extracted with dichloromethane (3×20 mL). The combined organic layers were washed with water, dried over Na₂SO₄, and concentrated under reduced pressure. The crude product was purified by preparative HPLC to obtain target compound (0.41 g, 20% yield).

¹H NMR: (400 MHz, DMSO-d₆) δ 10.49 (s, 1H), 9.67-9.52 (m, 1H), 9.45-9.28 (m, 1H), 8.12 (d, J=7.4 Hz, 1H) 8.01 (d, J=8.1 Hz, 1H), 7.64 (t, J=8.0 Hz, 1H), 4.90-4.78 (m, 1H), 3.85 (brs, 1H), 2.92-2.77 (m, 1H), 2.65-2.54 (m, 1H), 2.36-2.16 (m, 1H), 2.15-2.02 (m, 1H)

LCMS (m/z [M+H]*): 273.1

### Example 3: Synthesis of N-(2,6-dioxopiperidin-3-yl)-2-oxo-2,3-dihydro-1H-benzo[d]imidazole-4-carboxamide (3)

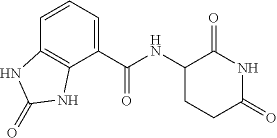 [SMILES: O=C1CCC(NC(=O)c2cccc3[nH]c(=O)[nH]c23)C(=O)N1]

This compound was synthesized using the general procedure shown in Reaction Scheme 1 and Synthetic Conditions B, above (14% yield), and 2-oxo-2,3-dihydro-1H-benzo[d]imidazole-4-carboxylic acid (20 mg) as a starting material. 1H NMR (500 MHz, DMSO) δ 10.89 (s, 1H), 10.83 (s, 1H), 10.19 (s, 1H), 8.74 (d, J=7.9 Hz, 1H), 7.44 (dd, J=8.1, 1.0 Hz, 1H), 7.09 (dd, J=7.6, 0.9 Hz, 1H), 7.02 (t, J=7.8 Hz, 1H), 4.84-4.74 (m, 1H), 2.82 (ddd, J=18.8, 13.4, 5.5 Hz, 1H), 2.60-2.54 (m, 1H), 2.16 (qd, J=13.0, 4.5 Hz, 1H), 2.00 (dddd, J=10.9, 8.2, 5.4, 2.9 Hz, 1H).

LCMS (m/z [M+H]⁺): 288.7

### Example 4: Synthesis of N-(2,6-dioxopiperidin-3-yl)-1-methyl-1H-benzo[d]imidazole-4-carboxamide (4)

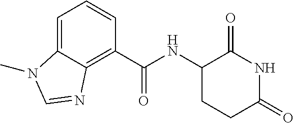 [SMILES: Cn1cnc2c(C(=O)NC3CCC(=O)NC3=O)cccc21]

This compound was synthesized using the general procedure shown in Reaction Scheme 1 and Synthetic Conditions B, above (25% yield), and 1-methyl-1H-benzo[d]imidazole-4-carboxylic acid (20 mg) as a starting material.

¹H NMR (500 MHz, DMSO) δ 10.93 (s, 1H), 10.19 (d, J=7.3 Hz, 1H), 8.47 (s, 1H), 7.94 (dd, J=7.5, 1.0 Hz, 1H), 7.85 (dd, J=8.1, 1.0 Hz, 1H), 7.44 (t, J=7.8 Hz, 1H), 4.91 (ddd, J=12.6, 7.2, 5.3 Hz, 1H), 3.94 (s, 3H), 2.83 (ddd, J=17.6, 13.5, 5.5 Hz, 1H), 2.61-2.53 (m, 1H), 2.26 (dtd, J=12.8, 5.4, 2.4 Hz, 1H), 2.11 (qd, J=12.9, 4.5 Hz, 1H).

LCMS (m/z [M+H]⁺): 286.4

### Example 5: Synthesis of N-(2,6-dioxopiperidin-3-yl)-1-methyl-1H-benzo[d]imidazole-7-carboxamide (5)

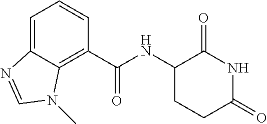 [SMILES: Cn1cnc2cccc(C(=O)NC3CCC(=O)NC3=O)c21]

This compound was synthesized using the general procedure shown in Reaction Scheme 1 and Synthetic Conditions B, above (4% yield), and 1-methyl-1H-benzo[d]imidazole-7-carboxylic acid (20 mg) as a starting material.

¹H NMR (500 MHz, DMSO) δ 10.87 (s, 1H), 8.94 (d, J=8.4 Hz, 1H), 8.29 (s, 1H), 7.84-7.71 (m, 1H), 7.37 (dt, J=7.4, 3.7 Hz, 1H), 7.28 (dd, J=8.0, 7.5 Hz, 1H), 4.80 (ddd, J=12.5, 8.4, 5.5 Hz, 1H), 3.87 (s, 3H), 2.83 (ddd, J=17.4, 13.1, 5.7 Hz, 1H), 2.56 (ddd, J=9.9, 5.2, 2.5 Hz, 1H), 2.15 (qd, J=12.9, 4.5 Hz, 1H), 2.07 (tdd, J=8.5, 5.6, 2.8 Hz, 1H).

LCMS (m/z [M+H]⁺): 286.7

### Example 6: Synthesis of N-(2,6-dioxopiperidin-3-yl)-5-hexanamido-1-methyl-1H-benzo[d]imidazole-7-carboxamide (6)

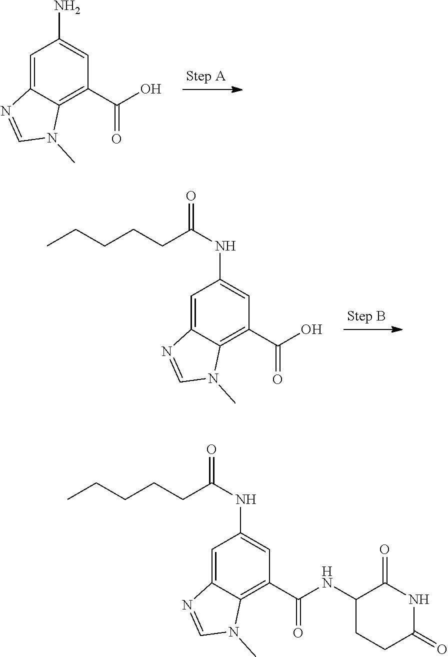 [SMILES: Cn1cnc2cc(N)cc(C(=O)O)c21 ; CCCCCC(=O)Nc1cc(C(=O)O)c2c(c1)ncn2C ; CCCCCC(=O)Nc1cc(C(=O)NC2CCC(=O)NC2=O)c2c(c1)ncn2C]

Step A: 5-amino-1-methyl-1H-benzo[d]imidazole-7-carboxylic acid dihydrochloride (20 mg, 0.076 mmol) and hexanoyl chloride (1.1 eq.) were dissolved in 4 mL of dry DCM and cooled in water/ice bath. TEA (4 eq.) was slowly injected into the reaction mixture. The ice bath was removed and the reaction was allowed to warm up to ambient temperature. The reaction was completed in two hours, monitored by LCMS. The solution was diluted with DCM (10 mL) and washed with 7 mL 3% HCl water soln. The aqueous phase was then evaporated to yield off-white crystals and 5-hexanamido-1-methyl-1H-benzo[d]imidazole-7-carboxylic acid was used directly in the next step.

Step B: This compound was synthesized using the general procedure shown in Reaction Scheme 1 and Synthetic Conditions B, above (29% yield), and 5-hexanamido-1-methyl-1H-benzo[d]imidazole-7-carboxylic acid (20 mg) as a starting material.

¹H NMR (500 MHz, DMSO) δ 10.87 (s, 1H), 10.00 (s, 1H), 8.97 (t, J=14.9 Hz, 1H), 8.21 (s, 1H), 8.16 (d, J=1.9 Hz, 1H), 7.51 (d, J=1.9 Hz, 1H), 4.79 (ddd, J=12.6, 8.4, 5.4 Hz, 1H), 3.82 (s, 3H), 2.82 (ddd, J=17.4, 13.1, 5.6 Hz, 1H), 2.57 (dt, J=16.6, 3.2 Hz, 1H), 2.31 (t, J=7.4 Hz, 2H), 2.20-2.09 (m, 1H), 2.09-2.01 (m, 1H), 1.67-1.56 (m, 2H), 1.37-1.25 (m, 4H), 0.87 (dt, J=7.1, 5.0 Hz, 3H).

LCMS (m/z [M+H]⁺): 400.2

### Example 7: Synthesis of N-(2,6-dioxopiperidin-3-yl)-5-fluoro-1H-benzo[d]imidazole-4-carboxamide (7)

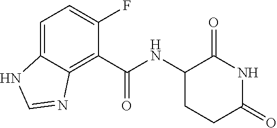 [SMILES: O=C1CCC(NC(=O)c2c(F)ccc3[nH]cnc23)C(=O)N1]

This compound was synthesized using the general procedure shown in Reaction Scheme 1 and Synthetic Conditions C, above (35% yield), and 5-fluoro-1H-benzo[d]imidazole-4-carboxylic acid (20 mg) as a starting material.

¹H NMR (500 MHz, DMSO) δ 12.66 (s, 1H), 10.90 (s, 1H), 8.30 (s, 1H), 7.79 (s, 1H), 7.17 (dd, J=11.9, 8.8 Hz, 1H), 4.84 (dd, J=17.6, 7.8 Hz, 1H), 2.90-2.74 (m, 1H), 2.59-2.53 (m, 1H), 2.25-2.07 (m, 2H).

LCMS (m/z [M+H]⁺): 291.3

### Example 8: Synthesis of N-(2,6-dioxopiperidin-3-yl)-6-fluoro-1H-benzo[d]imidazole-4-carboxamide (8)

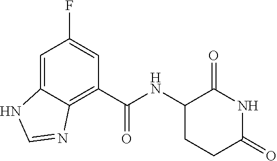 [SMILES: O=C1CCC(NC(=O)c2cc(F)cc3[nH]cnc23)C(=O)N1]

This compound was synthesized using the general procedure shown in Reaction Scheme 1 and Synthetic Conditions B, above (43% yield), and 6-fluoro-1H-benzo[d]imidazole-4-carboxylic acid (19.5 mg) as a starting material.

¹H NMR (500 MHz, DMSO) δ 13.09 (s, 1H), 10.94 (s, 1H), 10.25 (d, J=7.2 Hz, 1H), 8.51 (s, 1H), 7.64 (s, 1H), 7.62 (d, J=2.8 Hz, 1H), 4.91 (dt, J=12.4, 6.1 Hz, 1H), 2.83 (ddd, J=17.6, 13.5, 5.5 Hz, 1H), 2.55 (t, J=12.4 Hz, 1H), 2.31-2.19 (m, 1H), 2.11 (ddd, J=15.3, 12.0, 5.3 Hz, 1H).

LCMS (m/z [M+H]⁺): 290.9

### Example 9: Synthesis of N-(2,6-dioxopiperidin-3-yl)-3H-imidazo[4,5-b]pyridine-7-carboxamide (9)

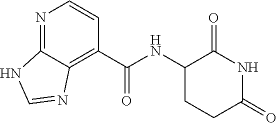 [SMILES: O=C1CCC(NC(=O)c2ccnc3[nH]cnc23)C(=O)N1]

This compound was synthesized using the general procedure shown in Reaction Scheme 1 and Synthetic Conditions C, above (76% yield), and 3H-imidazo[4,5-b]pyridine-7-carboxylic acid (20 mg) as a starting material.

¹H NMR (500 MHz, DMSO) δ δ 13.68 (s, 0.8H), 12.84 (s, 0.2H), 10.98 (s, 0.8H), 10.94 (s, 0.2H), 9.93 (d, J=7.3 Hz, 0.8H), 9.27 (d, J=8.2 Hz, 0.2H), 8.72 (s, 0.8H), 8.56 (d, J=5.1 Hz, 0.2 HH), 8.54 (d, J=5.0 Hz, 0.8H), 8.47 (s, 0.2H), 7.78 (d, J=5.0 Hz, 0.8H), 7.71 (d, J=5.0 Hz, 0.2H), 4.95 (ddd, J=12.6, 7.1, 5.4 Hz, 0.8H), 4.89-4.80 (m, 0.2H), 2.84 (ddd, J=17.6, 13.6, 5.5 Hz, 1H), 2.62-2.55 (m, 1H), 2.32-2.23 (m, 1H), 2.14 (ddd, J=26.3, 13.0, 4.6 Hz, 1H).

LCMS (m/z [M+H]⁺): 274.1

### Example 10: Synthesis of N-(2,6-dioxopiperidin-3-yl)-3H-imidazo[4,5-c]pyridine-7-carboxamide

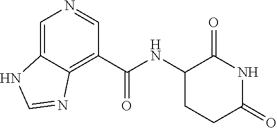 [SMILES: O=C1CCC(NC(=O)c2cncc3[nH]cnc23)C(=O)N1]

This compound was synthesized using the general procedure shown in Reaction Scheme 1 and Synthetic Conditions C, above (57% yield), and 3H-imidazo[4,5-c]pyridine-7-carboxylic acid (20 mg) as a starting material.

¹H NMR (500 MHz, DMSO, 353K) S 12.90 (s, 1H), 10.51 (s, 1H), 9.38 (s, 1H), 8.97 (s, 1H), 8.80 (s, 1H), 8.41 (s, 1H), 4.83-4.73 (m, 1H), 2.73 (ddd, J=18.5, 13.0, 5.6 Hz, 1H), 2.58-2.50 (m, 1H), 2.23-2.13 (m, 1H), 2.07 (ddd, J=25.6, 12.8, 4.6 Hz, 1H).

LCMS (m/z [M+H]⁺): 274.1

### Example 11: Synthesis of N-(2,6-dioxopiperidin-3-yl)-1H-imidazo[4,5-c]pyridine-4-carboxamide (11)

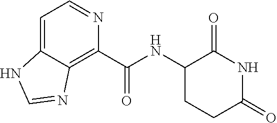 [SMILES: O=C1CCC(NC(=O)c2nccc3[nH]cnc23)C(=O)N1]

This compound was synthesized using the general procedure shown in Reaction Scheme 1 and Synthetic Conditions C, above (75% yield), and 1H-imidazo[4,5-c]pyridine-4-carboxylic acid (20 mg) as a starting material.

¹H NMR (500 MHz, DMSO) δ 12.93 (s, 1H), 10.80 (s, 1H), 9.14 (d, J=6.2 Hz, 1H), 8.38 (s, 1H), 8.31 (d, J=5.4 Hz, 1H), 7.83 (d, J=5.0 Hz, 1H), 4.81-4.67 (m, 1H), 2.79-2.67 (m, 1H), 2.47 (dd, J=17.3, 2.4 Hz, 1H), 2.21 (dd, J=22.4, 12.1 Hz, 1H), 2.04-1.92 (m, 1H).

LCMS (m/z [M+H]⁺): 273.9

### Example 12: Synthesis of 2-chloro-N-(2,6-dioxopiperidin-3-yl)-1H-benzo[d]imidazole-4-carboxamide (12)

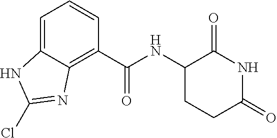 [SMILES: O=C1CCC(NC(=O)c2cccc3[nH]c(Cl)nc23)C(=O)N1]

This compound was synthesized using the general procedure shown in Reaction Scheme 1 and Synthetic Conditions B, above (13% yield), and 2-chloro-1H-benzo[d]imidazole-4-carboxylic acid (15 mg) as a starting material.

¹H NMR (500 MHz, DMSO) δ 13.85 (s, 1H), 10.92 (s, 2H), 9.66 (s, 2H), 7.86 (s, 2H), 7.70 (d, J=6.9 Hz, 3H), 7.36 (t, J=7.5 Hz, 3H), 4.86 (s, 3H), 2.89-2.76 (m, 3H), 2.61-2.53 (m, 4H), 2.16 (s, 6H).

LCMS (m/z [M+H]⁺): 307.0

### Example 13: Synthesis of N-(2,5-dioxopyrrolidin-3-yl)-2-methyl-1H-benzo[d]imidazole-4-carboxamide (14)

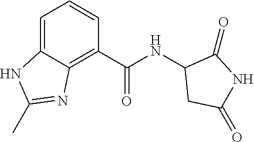 [SMILES: Cc1nc2c(C(=O)NC3CC(=O)NC3=O)cccc2[nH]1]

This compound was synthesized using the general procedure shown in Reaction Scheme 1 and Synthetic Conditions C, above, (70% yield) using 2-methyl-1H-benzo[d]imidazole-4-carboxylic acid (36 mg) and 3-aminopyrrolidine-2,5-dione hydrochloride salt (20.5 mg) as starting materials.

¹H NMR (500 MHz, DMSO, 353K) S 11.73 (s, 2H), 10.03 (s, 1H), 7.77 (d, J=7.6 Hz, 1H), 7.65 (d, J=7.8 Hz, 1H), 7.25 (t, J=7.8 Hz, 1H), 4.83-4.74 (m, 1H), 3.03 (dd, J=17.5, 9.2 Hz, 1H), 2.77 (dd, J=17.5, 5.7 Hz, 1H), 2.60 (s, 3H).

LCMS (m/z [M+H]⁺): 272.85

### Example 14: Synthesis of N-(2,6-dioxopiperidin-3-yl)-2-methyl-1H-benzo[d]imidazole-4-carboxamide (15)

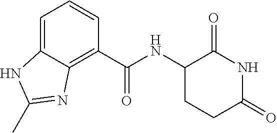 [SMILES: Cc1nc2c(C(=O)NC3CCC(=O)NC3=O)cccc2[nH]1]

This compound was synthesized using the general procedure shown in Reaction Scheme 1 and Synthetic Conditions B, above (20% yield), and 2-methyl-1H-benzo[d]imidazole-4-carboxylic acid (20 mg) as a starting material.

¹H NMR (500 MHz, DMSO) δ 12.73 (s, 1H), 10.90 (s, 1H), 10.29 (d, J=7.3 Hz, 1H), 7.82 (d, J=7.0 Hz, 1H), 7.63 (s, 1H), 7.32-7.23 (m, 1H), 4.87 (ddd, J=12.6, 7.1, 5.4 Hz, 1H), 2.89-2.76 (m, 1H), 2.58 (s, 3H), 2.55 (d, J=3.7 Hz, 1H), 2.28-2.19 (m, 1H), 2.18-2.07 (m, 1H).

LCMS (m/z [M+H]⁺): 286.5

### Example 15: Synthesis of methyl 2-(3-(2-methyl-1H-benzo[d]imidazole-4-carboxamido)-2,6-dioxopiperidin-1-yl)acetate (17)

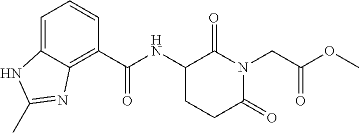

This compound was synthesized using the general procedure shown in Reaction Scheme 1 and Synthetic Conditions B, above, (31% yield) using 2-methyl-1H-benzo[d]imidazole-4-carboxylic acid (40 mg) and methyl 2-(3-amino-2,6-dioxopiperidin-1-yl)acetate (trifluoroacetic acid salt, 1.0 eq.) as starting materials.

¹H NMR (500 MHz, DMSO) δ 12.75 (s, 1H), 10.35 (s, 1H), 7.83 (s, 1H), 7.65 (d, J=4.6 Hz, 1H), 7.28 (d, J=5.7 Hz, 1H), 5.06 (d, J=5.3 Hz, 1H), 4.45 (s, 2H), 3.66 (s, 3H), 3.03 (t, J=15.4 Hz, 1H), 2.81 (d, J=16.9 Hz, 1H), 2.57 (t, J=11.7 Hz, 3H), 2.30 (s, 1H), 2.16 (d, J=12.9 Hz, 1H).

LCMS (m/z [M+H]⁺): 359.0

### Example 16: Synthesis of 2-methyl-N-(2-oxoazepan-3-yl)-1H-1,3-benzodiazole-4-carboxamide (19)

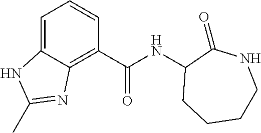 [SMILES: Cc1nc2c(C(=O)NC3CCCCNC3=O)cccc2[nH]1]

A vial was charged with 2-methyl-1H-1,3-benzodiazole-4-carboxylic acid (60.0 mg, 0.341 mmol, 1.000 eq), 3-aminoazepan-2-one hydrochloride (67.3 mg, 0.409 mmol, 1.200 eq), DMAP (4.2 mg, 0.034 mmol, 0.100 eq) and purged with Argon for 15 min. DMF (10 mL) added via syringe followed by DIPEA (0.119 mL, 0.681 mmol, 2.000 eq) and HATU (155.4 mg, 0.409 mmol, 1.200 eq) and the reaction mixture was stirred overnight. Solvent was evaporated under reduced pressure and the crude compound was purified by preparative TLC to provide 81 mg (82% yield) of the product.

¹H NMR (500 MHz, DMSO) δ 12.77 (s, 1H), 10.45 (s, 1H), 7.90-7.73 (m, 2H), 7.61 (dd, J=7.8, 0.7 Hz, 1H), 7.23 (t, J=7.8 Hz, 1H), 4.73 (ddd, J=10.9, 6.6, 1.3 Hz, 1H), 3.30-3.21 (m, 1H), 3.18-3.06 (m, 1H), 2.58 (s, 3H), 2.03-1.90 (m, 2H), 1.82-1.70 (m, 2H), 1.53 (dd, J=24.4, 11.9 Hz, 1H), 1.34-1.21 (m, 1H).

LCMS (m/z [M+H]⁺): 286.9

### Example 17: Synthesis of N-(2,7-dioxoazepan-3-yl)-2-methyl-1H-benzo[d]imidazole-4-carboxamide (20)

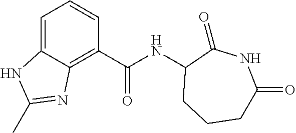 [SMILES: Cc1nc2c(C(=O)NC3CCCC(=O)NC3=O)cccc2[nH]1]

To a solution of 2-methyl-N-(2-oxoazepan-3-yl)-1H-1,3-benzodiazole-4-carboxamide (20.0 mg, 0.070 mmol, 1.000 eq) in MeCN (4.0 mL)/DMSO (0.085 mL)/water (0.010 mL) was added Dess-Martin periodinane (74.1 mg, 0.175 mmol, 2.500 eq). The suspension was heated at 80° C. for 1 h. Solvent was evaporated under reduced pressure and the crude product was purified by preparative TLC and HPLC to provide 16 mg (76%) of the product.

¹H NMR (500 MHz, DMSO) δ 12.73 (s, 1H), 10.67 (s, 1H), 10.38 (d, J=6.5 Hz, 1H), 7.81 (dd, J=7.6, 1.0 Hz, 1H), 7.64 (d, J=7.8 Hz, 1H), 7.27 (t, J=7.7 Hz, 1H), 5.19-5.06 (m, 1H), 3.08-2.95 (m, 1H), 2.65-2.61 (m, 1H), 2.60 (s, 3H), 2.35-2.22 (m, 1H), 2.08-1.94 (m, 1H), 1.89-1.69 (m, 2H).

LCMS (m/z [M+H]⁺): 301.1

### Example 18: Synthesis of 2-cyano-N-(2,6-dioxopiperidin-3-yl)-1H-benzo[d]imidazole-4-carboxamide (22)

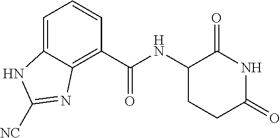 [SMILES: N#Cc1nc2c(C(=O)NC3CCC(=O)NC3=O)cccc2[nH]1]

This compound was synthesized using the general procedure shown in Reaction Scheme 1 and Synthetic Conditions B, above (59% yield), and 2-cyano-benzo[d]imidazole-4-carboxylic acid (20 mg) as a starting material.

¹H NMR (500 MHz, DMSO) δ 14.23 (s, 1H), 10.59 (s, 1H), 9.32 (s, 1H), 8.06 (d, J=7.4 Hz, 1H), 7.92 (d, J=8.2 Hz, 1H), 7.56 (t, J=7.7 Hz, 1H), 4.86 (dt, J=13.0, 7.2 Hz, 1H), 2.82 (ddd, J=18.5, 12.8, 5.9 Hz, 1H), 2.63 (dt, J=17.4, 3.7 Hz, 1H), 2.20 (ddd, J=25.4, 12.6, 4.5 Hz, 2H).

LCMS (m/z [M+H]⁺): 297.9

### Example 19: Synthesis of 2-(difluoromethyl)-N-(2,6-dioxopiperidin-3-yl)-1H-benzo[d]imidazole-7-carboxamide (23)

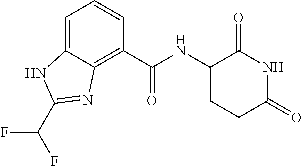 [SMILES: O=C1CCC(NC(=O)c2cccc3[nH]c(C(F)F)nc23)C(=O)N1]

This compound was synthesized using the general procedure shown in Reaction Scheme 1 and Synthetic Conditions B, above (86% yield), and 2-(difluoromethyl)-1H-benzo[d]imidazole-7-carboxylic acid (20 mg) as a starting material.

¹H NMR (500 MHz, DMSO) δ 13.89 (s, 1H), 10.93 (s, 1H), 9.89 (s, 1H), 7.97 (d, J=6.6 Hz, 1H), 7.83 (d, J=23.7 Hz, 1H), 7.48 (s, 1H), 7.44-7.17 (m, 1H), 4.88 (s, 1H).

LCMS (m/z [M+H]⁺): 323.3

### Example 20: Synthesis of N-(2,6-dioxopiperidin-3-yl)-2-isobutyl-1H-benzo[d]imidazole-7-carboxamide (24)

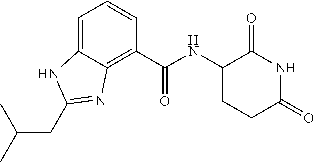 [SMILES: CC(C)Cc1nc2c(C(=O)NC3CCC(=O)NC3=O)cccc2[nH]1]

This compound was synthesized using the general procedure shown in Reaction Scheme 1 and Synthetic Conditions B, above (21% yield), and 2-isobutyl-1H-benzo[d]imidazole-7-carboxylic acid (20 mg) as a starting material.

¹H NMR (500 MHz, DMSO) δ 12.80 (s, 1H), 10.91 (s, 1H), 10.44 (s, 1H), 7.81 (d, J=6.1 Hz, 1H), 7.66 (d, J=7.8 Hz, 1H), 7.37-7.12 (m, 1H), 4.81 (d, J=43.5 Hz, 1H), 2.87-2.78 (m, 1H), 2.76 (td, J=7.2, 2.5 Hz, 3H), 2.61-2.54 (m, 1H), 2.33-2.18 (m, 1H), 2.17-2.03 (m, 1H), 1.02-0.90 (m, 6H).

LCMS (m/z [M+H]⁺): 329.0

### Example 21: Synthesis of N-(2,6-dioxopiperidin-3-yl)-2-(trifluoromethyl)-1H-benzo[d]imidazole-7-carboxamide (25)

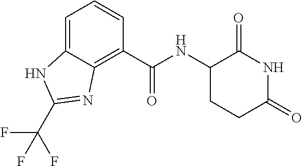 [SMILES: O=C1CCC(NC(=O)c2cccc3[nH]c(C(F)(F)F)nc23)C(=O)N1]

This compound was synthesized using the general procedure shown in Reaction Scheme 1 and Synthetic Conditions B, above (74% yield), and 2-(trifluoromethyl)-1H-benzo[d]imidazole-7-carboxylic acid (21 mg) as a starting material.

¹H NMR (500 MHz, DMSO) δ 14.58 (s, 1H), 10.94 (s, 1H), 9.74 (s, 1H), 8.03 (s, 1H), 7.90 (d, J=6.9 Hz, 1H), 7.58 (s, 1H), 4.88 (s, 1H), 2.89-2.76 (m, 1H), 2.57 (d, J=17.5 Hz, 1H), 2.29 (s, 1H), 2.20-2.08 (m, 1H).

LCMS (m/z [M+H]⁺): 340.9

### Example 22: Synthesis of 6-amino-N-(2,6-dioxopiperidin-3-yl)-2-(trifluoromethyl)-1H-1,3-benzodiazole-7-carboxamide (26)

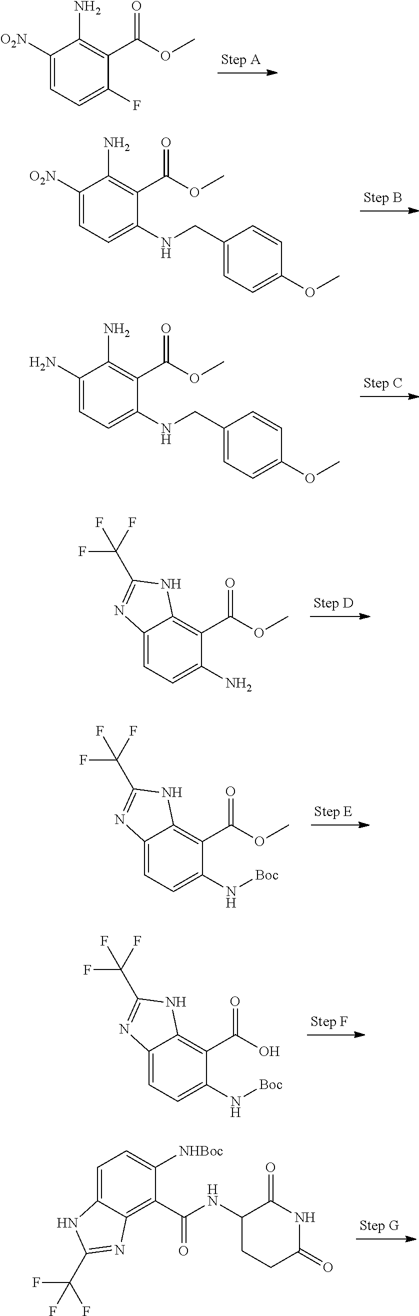

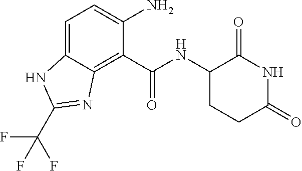 [SMILES: Nc1ccc2[nH]c(C(F)(F)F)nc2c1C(=O)NC1CCC(=O)NC1=O]

Step A: To a stirred solution of methyl 2-amino-6-fluoro-3-nitrobenzoate (2 g, 9.339 mmol) in DMSO (20 mL) was added K₂CO₃ (2.58 g, 18.67 mmol) followed by addition of (4-methoxyphenyl) methanamine (1.59 mL, 12.14 mmol). Then the reaction mixture was stirred at RT for 16 h. After completion of the reaction, quenched with ice water and precipitate was filtered and dried to give methyl 2-amino-6-((4-methoxybenzyl)amino)-3-nitrobenzoate 2.0 g (64% yield).

Step B: To a stirred solution of methyl 2-amino-6-((4-methoxybenzyl)amino)-3-nitrobenzoate (550 mg, 1.66 mmol) in THE (16 ml) was added Zn (1.5 g, 21.6 mmol) followed by addition of NH₄Cl (1.15 g, 21.6 mmol) in water (3 ml) at 0° C. and stirred at RT for 1 h. After completion of the reaction, reaction mixture was filtered through celite, washed with ethyl acetate. Organic layer was washed with water, brine, dried over sodium sulphate and concentrated under reduced pressure to give methyl 2,3-diamino-6-((4-methoxybenzyl)amino)benzoate (250 mg, crude) as brownish solid.

Step C: Methyl 2,3-diamino-6-((4-methoxybenzyl)amino)benzoate (2 g, 6.645 mmol) in TFA (20 mL) was stirred at rt for 16 h. After completion of the reaction, TFA was removed and quenched with aqueous NaHCO₃ and extracted with ethyl acetate. Organic layer washed with brine and dried over Na₂SO₄ and concentrated and purified by flash column chromatography to give methyl 6-amino-2-(trifluoromethyl)-1H-benzo[d]imidazole-7-carboxylate 200 mg (13% yield).

Step D: To a stirred solution of methyl 6-amino-2-(trifluoromethyl)-1H-benzo[d]imidazole-7-carboxylate (600 mg, 2.317 mmol) in dioxane (5 mL) was added aq NaOH (1N) (15 mL) followed by addition of Boc₂O (3.2 mL, 13.9 mmol) at 0° C. and stirred at RT for 72 h. After completion of the reaction quenched with ice water and extracted with ethyl acetate, dried over sodium sulphate and concentrated. The crude product was purified by flash column chromatography to give methyl 6-((tert-butoxycarbonyl)amino)-2-(trifluoromethyl)-1H-benzo[d]imidazole-7-carboxylate 600 mg (72% yield).

Step E: Solution of methyl 6-((tert-butoxycarbonyl)amino)-2-(trifluoromethyl)-1H-benzo[d]imidazole-7-carboxylate in 50% aq NaOH (13 mL) was stirred at 80° C. for 4 h. After completion of reaction, reaction mixture was acidified with 2M HCl and the precipitate was filtered to give 6-((tert-butoxycarbonyl)amino)-2-(trifluoromethyl)-1H-benzo[d]imidazole-7-carboxylic acid 300 mg (52% yield).

Step F: tert-butyl N-{7-[(2,6-dioxopiperidin-3-yl)carbamoyl]-2-(trifluoromethyl)-1H-1,3-benzodiazol-6-yl}carbamate was synthesized using the general procedure shown in Reaction Scheme 1 and Synthetic Conditions B, above (36% yield) using 5-((tert-butoxycarbonyl)amino)-2-(trifluoromethyl)-1H-benzo[d]imidazole-4-carboxylic acid (30.0 mg) as a starting material.

Step G: Tert-butyl (4-((2,6-dioxopiperidin-3-yl)carbamoyl)-2-(trifluoromethyl)-1H-benzo[d]imidazol-5-yl)carbamate (10.0 mg, 0.022 mmol, 1.000 eq) was dissolved in THE (0.220 mL) and 4M HCl in dioxane (0.038 mL, 1.098 mmol, 50.000 eq) was added. The mixture was stirring in RT for 4 h. Solvent was evaporated under reduced pressure to give 6-amino-N-(2,6-dioxopiperidin-3-yl)-2-(trifluoromethyl)-1H-1,3-benzodiazole-7-carboxamide hydrochloride 8.0 mg (88.0% yield).

1H NMR (500 MHz, DMSO) δ 14.15 (s, 1H), 10.91 (s, 1H), 10.19 (s, 1H), 7.54 (d, J=9.0 Hz, 1H), 6.94 (d, J=9.0 Hz, 1H), 4.86-4.77 (m, 1H), 2.88-2.75 (m, 1H), 2.63-2.54 (m, 1H), 2.33-2.22 (m, 1H), 2.10 (qd, J=12.9, 4.4 Hz, 1H).

LCMS (m/z [M+H]⁺): 356.3

### Example 23: Synthesis of 5-amino-N-(2,6-dioxopiperidin-3-yl)-2-(trifluoromethyl)-1H-benzo[d]imidazole-7-carboxamide (27)

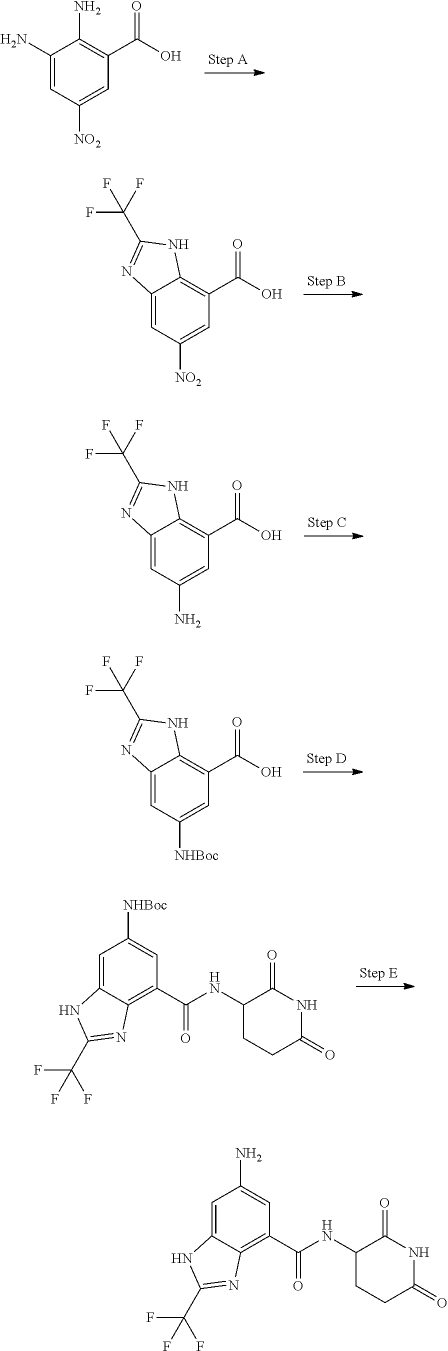 [SMILES: Nc1cc([N+](=O)[O-])cc(C(=O)O)c1N ; O=C(O)c1cc([N+](=O)[O-])cc2nc(C(F)(F)F)[nH]c12 ; Nc1cc(C(=O)O)c2[nH]c(C(F)(F)F)nc2c1 ; CC(C)(C)OC(=O)Nc1cc(C(=O)O)c2[nH]c(C(F)(F)F)nc2c1 ; CC(C)(C)OC(=O)Nc1cc(C(=O)NC2CCC(=O)NC2=O)c2nc(C(F)(F)F)[nH]c2c1 ; Nc1cc(C(=O)NC2CCC(=O)NC2=O)c2nc(C(F)(F)F)[nH]c2c1]

Step A: TFA (2 mL) and 4(N) HCl (5 mL) were added to 2,3-diamino-5-nitrobenzoic acid (500 mg, 2.54 mmol). Then the resulting reaction mixture was allowed to reflux for 12 h. After completion of reaction, the reaction mixture was cooled to 0° C. and then carefully neutralized with 10M NaOH solution. Aqueous part was extracted by DCM (100 mL×3). Organic layer was washed with brine and dried over Na₂SO₄ and concentrated to get the crude. Finally the crude was triturated with pentane and ether to get crude compound of 5-nitro-2-(trifluoromethyl)-1H-benzo[d]imidazole-7-carboxylic acid (500 mg) as dark brown solid. Compound was used in next step without further purification

Step B: To a stirred solution of 5-nitro-2-(trifluoromethyl)-1H-benzo[d]imidazole-7-carboxylic acid (500.0 mg, 1.82 mmol) in MeOH (10 mL) was added 10% Pd/C (193 mg). The reaction mixture was allowed to stir at rt for 4 h under hydrogen atmosphere. After completion of the reaction, the reaction mixture was filtered through celite and concentrated under reduced pressure to get methyl 5-amino-2-(trifluoromethyl)-1H-benzo[d]imidazole-7-carboxylic acid (500 mg) as crude which was used in next step without further purification.

Step C: To an ice cooled solution of methyl 5-amino-2-(trifluoromethyl)-1H-benzo[d]imidazole-7-carboxylic acid (1.0 g, 4.1 mmol) in dioxane (5.0 mL) and H₂O (5.0 mL) was added TEA (0.85 mL, 6.1 mmol). The reaction mixture was allowed to stir at ice cool condition for 2-3 min. Boc₂O (1.0 mL, 4.49 mmol) was added and the reaction mixture was stirred at RT for 6 h. After completion of reaction, solvent was evaporated and the crude product was purified by preparative HPLC to give 5-((tert-butoxycarbonyl)amino)-2-(trifluoromethyl)-1H-benzo[d]imidazole-7-carboxylic acid (50 mg) as white solid (2.8% yield over 3 steps).

Step D: Tert-butyl (7-((2,6-dioxopiperidin-3-yl)carbamoyl)-2-(trifluoromethyl)-1H-benzo[d]imidazol-5-yl)carbamate was synthesized using the general procedure shown in Reaction Scheme 1 and Synthetic Conditions B, above (37% yield) using 5-((tert-butoxycarbonyl)amino)-2-(trifluoromethyl)-1H-benzo[d]imidazole-7-carboxylic acid (30.0 mg) as a starting material.

Step E: Tert-butyl (7-((2,6-dioxopiperidin-3-yl)carbamoyl)-2-(trifluoromethyl)-1H-benzo[d]imidazol-5-yl)carbamate (10.0 mg, 0.022 mmol, 1.000 eq) was dissolved in THE (0.220 mL) and 4 M HCl in dioxane_(0.038 mL, 1.098 mmol, 50.000 eq) was added. The mixture was stirring in RT for 4 h. Solvent was evaporated under reduced pressure to give 5-amino-N-(2,6-dioxopiperidin-3-yl)-2-(trifluoromethyl)-1H-benzo[d]imidazole-7-carboxamide hydrochloride.

¹H NMR (500 MHz, DMSO) δ 13.67 (s, 1H), 10.91 (s, 1H), 9.71 (s, 1H), 7.48-7.34 (m, 1H), 6.86 (d, J=2.1 Hz, 1H), 5.53 (s, 1H), 4.84 (ddd, J=12.4, 7.0, 5.2 Hz, 2H), 2.80 (ddd, J=17.3, 13.5, 5.5 Hz, 1H), 2.59-2.52 (m, 1H), 2.32-2.21 (m, 1H), 2.15-2.03 (m, 1H).

LCMS (m/z [M+H]⁺): 355.9

### Example 24: Synthesis of 7-amino-N-(2,6-dioxopiperidin-3-yl)-2-(trifluoromethyl)-1H-benzo[d]imidazole-4-carboxamide (28)

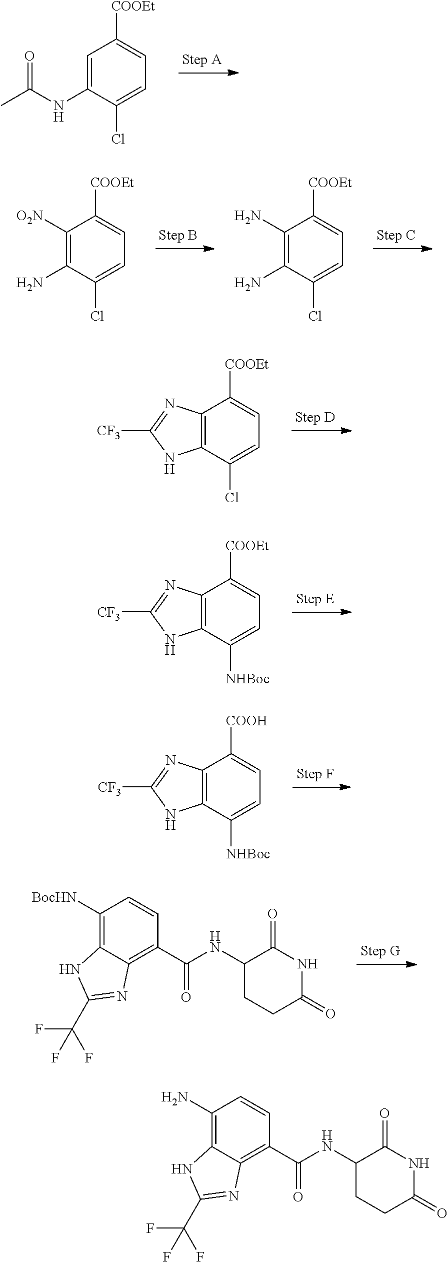

Step A: To ethyl 3-acetamido-4-chlorobenzoate (20.0 g, 82.97 mmol) was dropwise added 40.0 mL of 100% HNO₃ at −15° C. and the resultant reaction mixture was stirred and warmed up slowly to 10° C. during 2 h and then stirred at RT for 12 h, poured into crashed ice, the solids were filtered, dried under reduced pressure and the mixture of nitro compounds (16 g) was used directly in the next step. To a stirred solution of nitro compounds in 160 mL of ethanol was added 7.5 mL of conc. H₂SO₄. The reaction mixture was refluxed for 16 h, concentrated under reduced pressure and ice-cold water was added. The product was extracted into DCM, the combined organic layers were washed with brine, dried over Na₂SO₄ and concentrated. The crude product was purified by flash column chromatography to give ethyl 3-amino-4-chloro-2-nitrobenzoate (6.3 g, 30%).

Step B: To a stirred solution of ethyl 3-amino-4-chloro-2-nitrobenzoate (6.3 g, 25.753 mmol) in ethanol (60.0 mL) and water (30.0 mL) was added Fe powder (10.78 g) followed by NH₄Cl (1.791 g). The reaction mixture was refluxed for 12 h, concentrated under reduced pressure, diluted with DCM, filtered through celite bed and concentrated under reduced pressure. The crude product was purified by flash column chromatography to give ethyl 2,3-diamino-4-chlorobenzoate (5 g, 90.45%).

Step C: To ethyl 2,3-diamino-4-chlorobenzoate (2.0 g, 9.317 mmol, 1.0 eq) was added 15 ml of TFA and the reaction mixture was refluxed for 12 h and concentrated under reduced pressure. To the residue was added NaHCO₃ solution and the product was extracted with ethyl acetate, washed with brine, dried over Na₂SO₄ and concentrated. The crude product was purified by flash column chromatography to give ethyl 7-chloro-2-(trifluoromethyl)-1H-benzo[d]imidazole-4-carboxylate (2.4 g, 88% yield).

Step D: A solution of ethyl 7-chloro-2-(trifluoromethyl)-1H-benzo[d]imidazole-4-carboxylate (1.0 g, 3.417 mmol) in dioxane (12 mL) was degassed under argon atmosphere for 10-15 min. Cs₂CO₃ (2.22 g, 6.834 mmol), NH₂Boc (1.60 g, 13.669 mmol), X-phos (326 mg, 0.683 mmol) and X-phosPdG3 (0.289 g, 0.342 mmol) were added and reaction mixture was stirred at 85° C. for 16 h. Reaction mixture was filtered through celite bed, concentrated and purified by flash column chromatography to give ethyl 7-((tert-butoxycarbonyl)amino)-2-(trifluoromethyl)-1H-benzo[d]imidazole-4-carboxylate (800 mg, 62% yield).

Step E: A stirred solution of ethyl 7-((tert-butoxycarbonyl)amino)-2-(trifluoromethyl)-1H-benzo[d]imidazole-4-carboxylate (500.0 mg, 1.339 mmol) in MeOH (3.0 mL) and THE (3.0 mL) was added slowly 50% aqueous NaOH solution (6.0 mL) at ice cool condition. Then the resultant reaction mixture was allowed to stir at rt for 16 h. Reaction mixture was concentrated under reduced pressure and then it was diluted with water and washed with ethyl acetate. After that the aqueous part was gently neutralized with saturated aqueous citric acid solution in ice cool condition and then it was extracted with ethyl acetate. Then the combined organic layer was washed with brine and then dried over Na₂SO₄, filtered and concentrated to get the crude which was triturated with pentane and ether to get 7-((tert-butoxycarbonyl)amino)-2-(trifluoromethyl)-1H-benzo[d]imidazole-4-carboxylic acid (250 mg, 54.06% yield) as white solid.

Step F: Tert-butyl (4-((2,6-dioxopiperidin-3-yl)carbamoyl)-2-(trifluoromethyl)-1H-benzo[d]imidazol-7-yl)carbamate was synthesized using the general procedure shown in Reaction Scheme 1 and Synthetic Conditions B, above (80% yield), and 7-((tert-butoxycarbonyl)amino)-2-(trifluoromethyl)-1H-benzo[d]imidazole-4-carboxylic acid (30 mg) as a starting material. 1H NMR (500 MHz, DMSO) δ 14.02 (s, 1H), 10.93 (s, 1H), 9.57 (s, 1H), 8.93 (s, 1H), 7.98 (s, 2H), 4.86 (dt, J=12.3, 5.9 Hz, 1H), 2.88-2.79 (m, 1H), 2.57 (s, 1H), 2.29 (d, J=12.4 Hz, 1H), 2.11 (td, J=13.1, 4.5 Hz, 1H), 1.53 (s, 9H).

LCMS (m/z [M+H]⁺): 456.5

Step G: To the mixture of tert-butyl (4-((2,6-dioxopiperidin-3-yl)carbamoyl)-2-(trifluoromethyl)-1H-benzo[d]imidazol-7-yl)carbamate (8 mg, 0.018 mmol) in DCM (0.5 mL) was added TFA (0.1 mL) and the reaction mixture was stirred at RT for 18 h. The mixture was concentrated under reduced pressure and was purified by HPLC to give 7-amino-N-(2,6-dioxopiperidin-3-yl)-2-(trifluoromethyl)-1H-1,3-benzodiazole-4-carboxamide trifluoroacetate (44% yield).

¹H NMR (500 MHz, DMSO) δ 10.51 (s, 1H), 7.75 (d, J=8.3 Hz, 1H), 6.58 (s, 1H), 5.97 (d, J=72.1 Hz, 2H), 4.76 (d, J=10.7 Hz, 1H), 2.81-2.73 (m, 1H), 2.60 (dd, J=17.5, 3.9 Hz, 1H), 2.12 (d, J=26.4 Hz, 2H).

LCMS (m/z [M+H]⁺): 356.0

### Example 25: Synthesis of N-(2,6-dioxopiperidin-3-yl)-1,2-dimethyl-1H-benzo[d]imidazole-4-carboxamide (29)

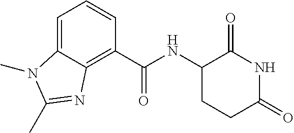 [SMILES: Cc1nc2c(C(=O)NC3CCC(=O)NC3=O)cccc2n1C]

This compound was synthesized using the general procedure shown in Reaction Scheme 1 and Synthetic Conditions B, above (10% yield), and 1,2-dimethyl-1H-benzo[d]imidazole-4-carboxylic acid (8.9 mg) as a starting material.

¹H NMR (500 MHz, DMSO) δ 10.91 (s, 1H), 10.25 (d, J=7.3 Hz, 1H), 7.85 (dd, J=7.6, 1.0 Hz, 1H), 7.75 (dd, J=8.0, 1.0 Hz, 1H), 7.33 (t, J=7.8 Hz, 1H), 4.87 (ddd, J=12.6, 7.2, 5.3 Hz, 1H), 3.81 (s, 3H), 2.82 (ddd, J=17.5, 13.5, 5.5 Hz, 1H), 2.62 (s, 3H), 2.56 (ddd, J=17.4, 4.1, 2.3 Hz, 1H), 2.24 (dtd, J=12.9, 5.4, 2.4 Hz, 1H), 2.18-2.07 (m, 1H).

LCMS (m/z [M+H]⁺): 301.0

### Example 26: Synthesis of N-(2,6-dioxopiperidin-3-yl)-6-methoxy-2-methyl-1H-benzo[d]imidazole-4-carboxamide (30)

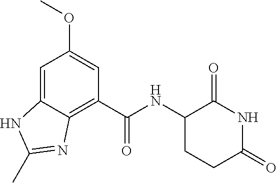 [SMILES: COc1cc(C(=O)NC2CCC(=O)NC2=O)c2nc(C)[nH]c2c1]

This compound was synthesized using the general procedure shown in Reaction Scheme 1 and Synthetic Conditions B, above (49% yield), and 6-methoxy-2-methyl-1H-benzo[d]imidazole-4-carboxylic acid (22 mg) as a starting material.

¹H NMR (500 MHz, DMSO) δ 12.52 (s, 1H), 10.90 (s, 1H), 10.26 (d, J=7.3 Hz, 1H), 7.39 (d, J=2.5 Hz, 1H), 7.16 (d, J=2.5 Hz, 1H), 4.86 (ddd, J=12.6, 7.3, 5.4 Hz, 1H), 3.82 (s, 3H), 2.87-2.76 (m, 1H), 2.54 (s, 3H), 2.53-2.51 (m, 1H), 2.26-2.19 (m, 1H), 2.12 (qd, J=12.9, 4.5 Hz, 1H).

LCMS (m/z [M+H]⁺): 317.5

### Example 27: Synthesis of 6-(aminomethyl)-N-(2,6-dioxopiperidin-3-yl)-2-methyl-1H-benzo[d]imidazole-4-carboxamide (31)

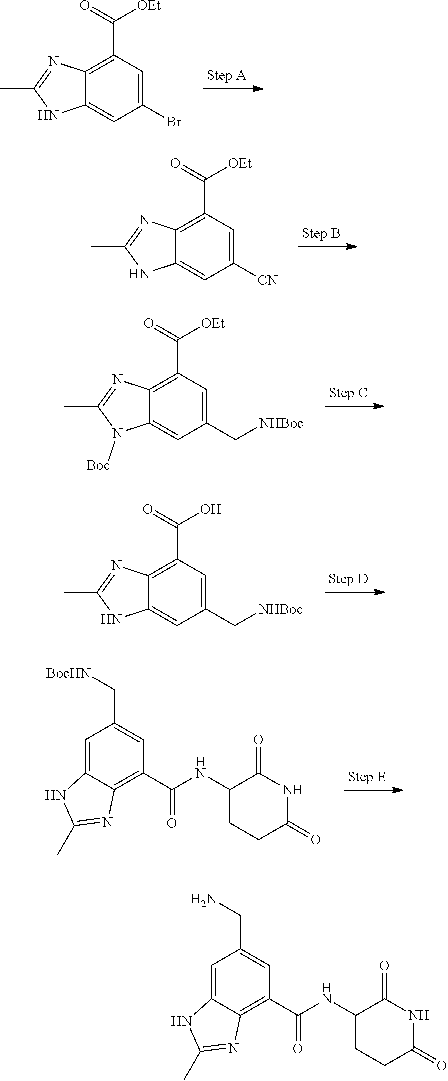

Step A: To a degassed solution of ethyl 6-bromo-2-methyl-1H-benzo[d]imidazole-4-carboxylate (500 mg, 1.76 mmol) in DMF (12 mL) were added ZN(CN)₂ (518 mg, 4.41 mmol) and Pd(PPh₃)₄(408 mg, 0.35 mmol) and the reaction mixture was at 120° C. for 16 h, quenched with ice water, extracted with ethyl acetate, dried over Na₂SO₄, concentrated under reduced pressure and purified by flash column chromatography to give ethyl 6-cyano-2-methyl-1H-benzo[d]imidazole-4-carboxylate (27% yield).

Step B: To a solution of ethyl 6-cyano-2-methyl-1H-benzo[d]imidazole-4-carboxylate (400 mg, 1.747 mmol) in ethanol (13 ml) were added Raney-nickel and Boc₂O (2.1 ml, 8.734 mmol) and the reaction mixture was stirred under hydrogen (15 psi) for 16 h, filtered through celite bed, filtrates were concentrated under reduced pressure and purified by flash column chromatography to give 1-(tert-butyl) 4-ethyl 6-(((tert-butoxycarbonyl)amino)methyl)-2-methyl-1H-benzo[d]imidazole-1,4-dicarboxylate (47% yield).

Step C: To a solution of 1-(tert-butyl) 4-ethyl 6-(((tert-butoxycarbonyl)amino)methyl)-2-methyl-1H-benzo[d]imidazole-1,4-dicarboxylate (430 mg, 0.993 mmol) in THF:MeOH 1:1 (10 mL) was added 50% aqueous NaOH (4 mL) and the reaction mixture was stirred at RT for 16 h, neutralized with 1M HCl, and filtered. The solids were dried to give 6-(((tert-butoxycarbonyl)amino)methyl)-2-methyl-1H-benzo[d]imidazole-4-carboxylic acid (62% yield).

Step D: Tert-butyl ((4-((2,6-dioxopiperidin-3-yl)carbamoyl)-2-methyl-1H-benzo[d]imidazol-6-yl)methyl)carbamate was synthesized using the general procedure shown in Reaction Scheme 1 and Synthetic Conditions B, above (45% yield), and 6-(((tert-butoxycarbonyl)amino)methyl)-2-methyl-1H-benzo[d]imidazole-4-carboxylic acid (30 mg) as a starting material.

¹H NMR (500 MHz, DMSO) δ 12.64 (s, 1H), 10.89 (s, 1H), 10.24 (d, J=7.3 Hz, 1H), 8.16 (s, 1H), 7.74 (s, 1H), 7.49 (s, 1H), 7.45 (t, J=6.4 Hz, 1H), 4.88 (dt, J=12.6, 6.4 Hz, 1H), 4.24 (d, J=6.2 Hz, 2H), 2.82 (ddd, J=17.3, 13.3, 5.5 Hz, 1H), 2.61-2.52 (m, 4H), 2.27-2.20 (m, 1H), 2.11 (qd, J=12.9, 4.3 Hz, 1H), 1.40 (s, 9H).

LCMS (m/z [M+H]⁺): 416.0

Step E: Tert-butyl ((4-((2,6-dioxopiperidin-3-yl)carbamoyl)-2-methyl-1H-benzo[d]imidazol-6-yl)methyl)carbamate was suspended in DCM (0.5 mL). To the mixture was added TFA (0.1 mL) and stirred for 2 h at RT. The crude was concentrated in vacuo, dissolved in water and freeze-dried to give 6-(aminomethyl)-N-(2,6-dioxopiperidin-3-yl)-2-methyl-1H-benzo[d]imidazole-4-carboxamide.

¹H NMR (500 MHz, DMSO) δ 10.93 (s, 1H), 10.12 (s, 1H), 8.14 (s, 3H), 7.97 (d, J=1.6 Hz, 1H), 7.79 (s, 1H), 4.88 (dt, J=13.0, 7.1 Hz, 1H), 4.20 (q, J=5.8 Hz, 2H), 2.84 (ddd, J=17.3, 13.0, 6.0 Hz, 1H), 2.67-2.53 (m, 4H), 2.25-2.09 (m, 2H).

LCMS (m/z [M+H]⁺): 315.8

### Example 28: Synthesis of 7-(aminomethyl)-N-(2,6-dioxopiperidin-3-yl)-2-methyl-1H-benzo[d]imidazole-4-carboxamide (32)

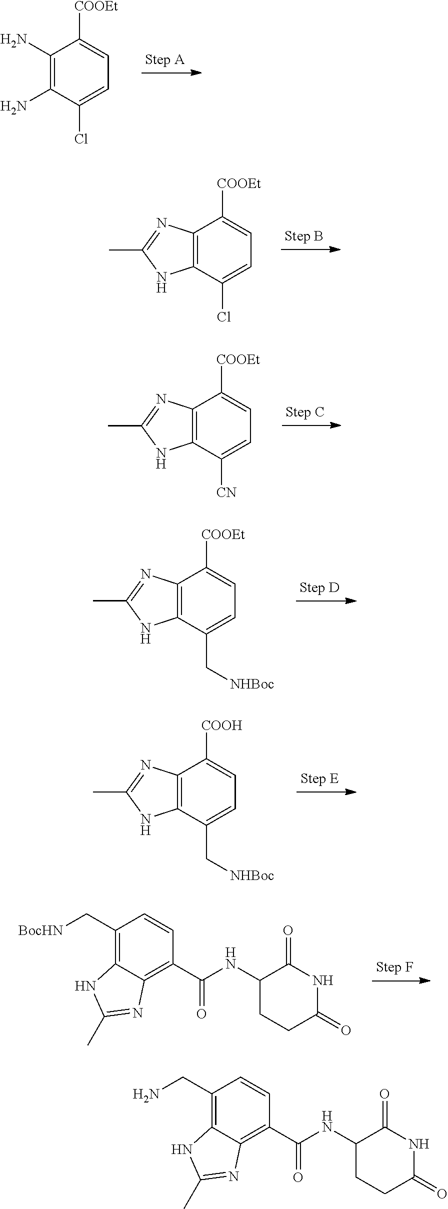

Step A: To a stirred solution of ethyl 2,3-diamino-4-chlorobenzoate (1.5 g, 6.99 mmol) in toluene (20.0 mL) was added respectively triethyl orthoacetate (5.1 mL, 27.95 mmol) and PTSA (0.337 g, 1.957 mmol) and the reaction mixture was refluxed for 16 h, concentrated under reduced pressure and the crude product was purified by flash column chromatography to give ethyl 7-chloro-2-methyl-1H-benzo[d]imidazole-4-carboxylate 1.2 g (71% yield).

Step B: A solution of ethyl 7-chloro-2-methyl-1H-benzo[d]imidazole-4-carboxylate (400 mg, 1.676 mmol) in DMF (10 mL) was degassed under argon atmosphere for 10-15 minutes. Zn(CN)₂ (492 mg, 4.19 mmol), X-phos (159.792 mg, 0.335 mmol) and X-phosPdG3 (0141.86 mg, 0.168 mmol) were added and the reaction mixture was heated to 110° C. for 16 h. The mixture was filtered through celite bed, diluted with water, the product was extracted with ethyl acetate, washed with brine, dried over Na₂SO₄ and concentrated under reduced pressure. The crude product was purified by flash column chromatography to give ethyl 7-cyano-2-methyl-1H-benzo[d]imidazole-4-carboxylate 251 mg (65% yield).

Step C: The a stirred solution of ethyl 7-cyano-2-methyl-1H-benzo[d]imidazole-4-carboxylate (3) (375 mg, 1.636 mmol) in ethanol (10 mL) was added Boc₂O (0.564 mL, 2.454 mmol) and Raney-nickel (200 mg) and reaction mixture was stirred at RT under hydrogen atmosphere for 16 h, filtered through celite bed and concentrated under reduced pressure. The crude product was purified by flash column chromatography to give ethyl 7-(((tert-butoxycarbonyl)amino)methyl)-2-methyl-1H-benzo[d]imidazole-4-carboxylate 230 mg (42% yield).

Step D: To a solution of ethyl 7-(((tert-butoxycarbonyl)amino)methyl)-2-methyl-1H-benzo[d]imidazole-4-carboxylate (200.0 mg, 0.6 mmol) in MeOH (1 mL) and THE (1 mL) was added 50% NaOH solution (2 mL) at 0° C. The reaction mixture was stirred at RT for 16 h, concentrated under reduced pressure, diluted with water and washed with DCM. The aqueous phase was gently acidified by citric acid solution and the product was extracted with ethyl acetate, washed with brine, dried over Na₂SO₄ and concentrated under reduced pressure. The crude product was triturated with diethyl ether to give 7-(((tert-butoxycarbonyl)amino)methyl)-2-methyl-1H-benzo[d]imidazole-4-carboxylic acid 60 mg (32%).

Step E: Tert-butyl ((4-((2,6-dioxopiperidin-3-yl)carbamoyl)-2-methyl-1H-benzo[d]imidazol-7-yl)methyl)carbamate was synthesized using the general procedure shown in Reaction Scheme 1 and Synthetic Conditions B, above (47% yield), and 7-(((tert-butoxycarbonyl)amino)methyl)-2-methyl-1H-benzo[d]imidazole-4-carboxylic acid (20 mg) as a starting material.

¹H NMR (500 MHz, DMSO) δ 12.66 (s, 1H), 10.89 (s, 1H), 10.24 (d, J=7.3 Hz, 1H), 8.15 (s, 1H), 7.79 (d, J=7.8 Hz, 1H), 7.47 (t, J=6.1 Hz, 1H), 7.13 (d, J=7.9 Hz, 1H), 4.86 (ddd, J=12.5, 7.2, 5.2 Hz, 1H), 4.42 (d, J=6.1 Hz, 2H), 2.81 (ddd, J=17.3, 13.5, 5.5 Hz, 1H), 2.61-2.51 (m, 4H), 2.26-2.20 (m, 1H), 2.16-2.07 (m, 1H), 1.40 (s, 9H).

LCMS (m/z [M+H]⁺): 416.0

Step F: Tert-butyl ((4-((2,6-dioxopiperidin-3-yl)carbamoyl)-2-methyl-1H-benzo[d]imidazol-7-yl)methyl)carbamate was suspended in DCM (0.5 mL). To the mixture was added TFA (0.1 mL) and stirred for 2 h at RT. The crude was concentrated in vacuo, dissolved in water and freeze-dried to give 7-(aminomethyl)-N-(2,6-dioxopiperidin-3-yl)-2-methyl-1H-benzo[d]imidazole-4-carboxamide.

¹H NMR (500 MHz, DMSO) δ 10.91 (s, 1H), 10.12 (s, 1H), 9.20 (s, 1H), 8.30 (s, 3H), 7.85 (d, J=7.8 Hz, 1H), 7.37 (d, J=7.9 Hz, 1H), 4.82 (d, J=10.7 Hz, 1H), 4.39 (d, J=5.7 Hz, 2H), 2.88-2.77 (m, 1H), 2.64 (s, 3H), 2.62-2.50 (m, 1H), 2.17 (s, 2H).

LCMS (m/z [M+H]⁺): 316.1

### Example 29: Synthesis of 5-(2,4-dimethoxyphenyl)-N-(2,6-dioxopiperidin-3-yl)-2-methyl-3H-imidazo[4,5-b]pyridine-7-carboxamide (33)

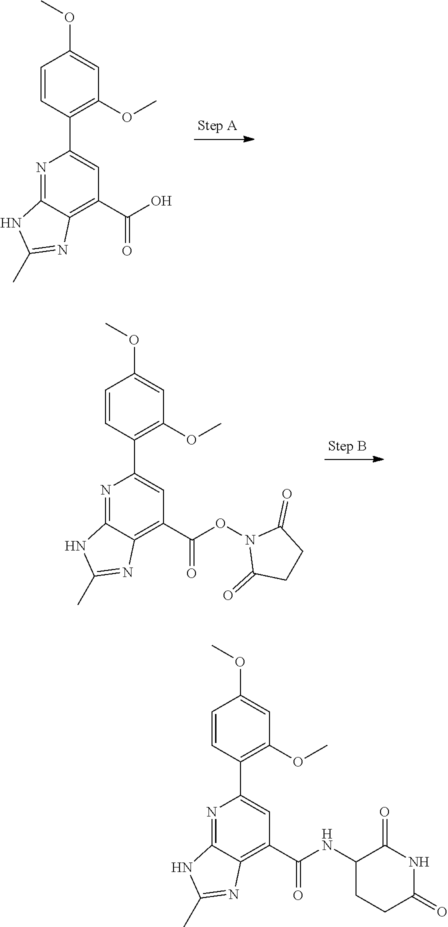

Step A: To a suspension of 5-(2,4-dimethoxyphenyl)-2-methyl-1H-imidazo[4,5-b]pyridine-7-carboxylic acid (10.0 mg, 31.917 μmol, 1.000 eq) and HOSu (4.4 mg, 38.300 μmol, 1.200 eq) in DCM (1.0 mL) was added a solution of DCC (7.9 mg, 38.300 μmol, 1.200 eq) in DCM (0.500 mL). The reaction mixture was stirred at RT for 18 h. The reaction mixture was concentrated under reduced pressure and purified by preparative TLC to give 2,5-dioxopyrrolidin-1-yl 5-(2,4-dimethoxyphenyl)-2-methyl-1H-imidazo[4,5-b]pyridine-7-carboxylate (71% yield).

Step B: To a solution of 3-aminopiperidine-2,6-dione hydrochloride (8.4 mg, 51.171 μmol, 3.000 eq) and DIPEA (9 μL, 51.171 μmol, 3.000 eq) in DMF (2.0 mL) was added 2,5-dioxopyrrolidin-1-yl 5-(2,4-dimethoxyphenyl)-2-methyl-1H-imidazo[4,5-b]pyridine-7-carboxylate (7.0 mg, 17.057 μmol, 1.000 eq) in one portion. The reaction mixture was stirred at RT for 18 h. The solvent was evaporated under reduced pressure and the residue was purified by preparative TLC to provide 4.1 mg (56%) of product.

¹H NMR (500 MHz, DMSO) δ 13.04 (s, 1H), 10.54 (s, 1H), 8.72 (d, J=8.0 Hz, 1H), 7.76 (d, J=8.5 Hz, 1H), 7.65 (s, 1H), 6.73 (d, J=2.4 Hz, 1H), 6.70 (dd, J=8.6, 2.4 Hz, 1H), 4.81 (q, J=8.2 Hz, 1H), 3.87 (s, 3H), 3.87 (s, 3H), 2.81 (dt, J=18.0, 9.5 Hz, 1H), 2.67-2.57 (m, 1H), 2.53 (s, 3H), 2.15 (dq, J=9.1, 5.2, 4.1 Hz, 2H).

LCMS (m/z [M+H]⁺): 423.9

### Example 30: Synthesis of N-(2,6-dioxopiperidin-3-yl)-2-methyl-3H-imidazo[4,5-c]pyridine-7-carboxamide (35)

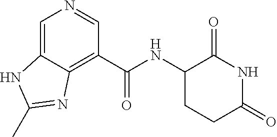 [SMILES: Cc1nc2c(C(=O)NC3CCC(=O)NC3=O)cncc2[nH]1]

This compound was synthesized using the general procedure shown in Reaction Scheme 1 and Synthetic Conditions C, above (60% yield), and 2-methyl-3H-imidazo[4,5-c]pyridine-7-carboxylic acid (20 mg) as a starting material.

¹H NMR (500 MHz, DMSO, 353K) S 12.65 (s, 1H), 10.58 (s, 1H), 9.50 (s, 1H), 8.89 (s, 1H), 8.82 (s, 1H), 8.14 (s, OH), 4.85 (dt, J=12.6, 6.9 Hz, 1H), 2.81 (ddd, J=17.5, 12.9, 5.7 Hz, 1H), 2.63 (s, 2H), 2.62-2.57 (m, OH), 2.25 (s, 1H), 2.22-2.11 (m, 1H).

LCMS (m/z [M+H]⁺): 288.1

### Example 31: Synthesis of N-(2,6-dioxopiperidin-3-yl)-2-methyl-3H-imidazo[4,5-b]pyridine-7-carboxamide (36)

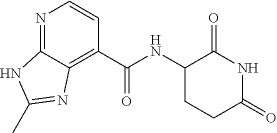 [SMILES: Cc1nc2c(C(=O)NC3CCC(=O)NC3=O)ccnc2[nH]1]

This compound was synthesized using the general procedure shown in Reaction Scheme 1 and Synthetic Conditions C, above (29% yield), and 2-methyl-3H-imidazo[4,5-b]pyridine-7-carboxylic acid (20 mg) as a starting material.

¹H NMR (500 MHz, DMSO, 353K) S 12.93 (s, 1H), 10.60 (s, 1H), 9.71 (s, 1H), 8.38 (d, J=5.0 Hz, 1H), 7.66 (d, J=5.0 Hz, 1H), 4.86 (ddd, J=12.4, 7.3, 5.3 Hz, 1H), 2.81 (ddd, J=17.3, 13.1, 5.5 Hz, 1H), 2.65-2.57 (m, 4H), 2.29 (dtd, J=10.7, 5.2, 2.7 Hz, 1H), 2.15 (qd, J=12.8, 4.7 Hz, 1H).

LCMS (m/z [M+H]⁺): 287.6

### Example 32: Synthesis of N-(2,6-dioxopiperidin-3-yl)-1H-pyrrolo[3,2-b]pyridine-7-carboxamide (37)

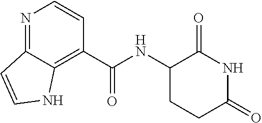 [SMILES: O=C1CCC(NC(=O)c2ccnc3cc[nH]c23)C(=O)N1]

This compound was synthesized using the general procedure shown in Reaction Scheme 1 and Synthetic Conditions C, above (30% yield), and 1H-pyrrolo[3,2-b]pyridine-7-carboxylic acid (20 mg) as a starting material.

¹H NMR (500 MHz, DMSO) δ 11.36 (s, 1H), 10.93 (s, 1H), 9.16 (d, J=8.3 Hz, 1H), 8.47 (d, J=4.9 Hz, 1H), 7.68-7.61 (m, 1H), 7.56 (d, J=5.0 Hz, 1H), 6.64 (d, J=3.0 Hz, 1H), 4.84 (ddd, J=12.6, 8.2, 5.4 Hz, 1H), 2.85 (ddd, J=17.4, 13.4, 5.5 Hz, 1H), 2.60 (ddd, J=17.3, 4.3, 2.9 Hz, 1H), 2.23 (qd, J=13.0, 4.5 Hz, 1H), 2.05 (dddd, J=10.8, 8.2, 5.4, 2.8 Hz, 1H).

LCMS (m/z [M+H]⁺): 272.9

### Example 33: Synthesis of N-(2,6-dioxopiperidin-3-yl)-2-methyl-1H-pyrrolo[2,3-c]pyridine-7-carboxamide (38)

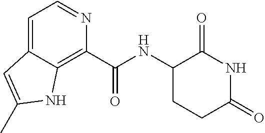 [SMILES: Cc1cc2ccnc(C(=O)NC3CCC(=O)NC3=O)c2[nH]1]

This compound was synthesized using the general procedure shown in Reaction Scheme 1 and Synthetic Conditions B, above (39% yield), and 2-methyl-1H-pyrrolo[2,3-c]pyridine-7-carboxylic acid (10 mg) as a starting material.

¹H NMR (500 MHz, DMSO) δ 11.43 (s, 1H), 10.89 (s, 1H), 9.13 (d, J=8.2 Hz, 1H), 8.14 (d, J=5.2 Hz, 1H), 7.65 (d, J=5.2 Hz, 1H), 6.39-6.29 (m, 1H), 4.85-4.75 (m, J=13.4, 8.1, 5.5 Hz, 1H), 2.84 (ddd, J=17.4, 13.8, 5.5 Hz, 1H), 2.61-2.56 (m, J=17.8, 3.1 Hz, 1H), 2.51 (s, 3H), 2.37-2.27 (m, J=13.1, 4.5 Hz, 1H), 2.14-2.04 (m, 1H).

LCMS (m/z [M+H]⁺): 287.1

### Example 34: Synthesis of N-(2,6-dioxopiperidin-3-yl)-2-methylbenzofuran-7-carboxamide (39)

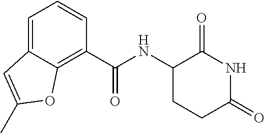 [SMILES: Cc1cc2cccc(C(=O)NC3CCC(=O)NC3=O)c2o1]

This compound was synthesized using the general procedure shown in Reaction Scheme 1 and Synthetic Conditions B, above (62% yield), and 2-methyl-1-benzofuran-7-carboxylic acid (20 mg) as a starting material.

¹H NMR (500 MHz, DMSO) δ 10.91 (s, 1H), 8.52 (d, J=7.6 Hz, 1H), 7.72 (dd, J=7.7, 1.3 Hz, 1H), 7.67 (dd, J=7.6, 1.3 Hz, 1H), 7.30 (t, J=7.6 Hz, 1H), 6.71 (q, J=1.1 Hz, 1H), 4.83 (ddd, J=12.1, 7.6, 5.7 Hz, 1H), 2.82 (ddd, J=17.3, 13.1, 5.9 Hz, 1H), 2.56 (ddd, J=17.3, 4.4, 2.8 Hz, 1H), 2.51 (s, 3H), 2.24-2.10 (m, 2H).

LCMS (m/z [M+H]⁺): 287.1

### Example 35: Synthesis of N-(2,6-dioxopiperidin-3-yl)-6,7,8,9-tetrahydrodibenzo[b,d]furan-4-carboxamide (40)

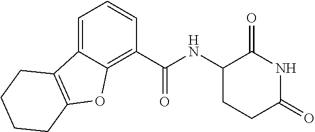 [SMILES: O=C1CCC(NC(=O)c2cccc3c4c(oc23)CCCC4)C(=O)N1]

This compound was synthesized using the general procedure shown in Reaction Scheme 1 and Synthetic Conditions B, using COMU instead of HATU, above (45.5% yield), and 6,7,8,9-tetrahydrodibenzo[b,d]furan-4-carboxylic acid (20 mg) as a starting material.

NMR: 1H NMR (500 MHz, DMSO) δ 10.90 (s, 1H), 8.50 (d, J=7.6 Hz, 1H), 7.67 (ddd, J=12.0, 7.7, 1.3 Hz, 2H), 7.32 (t, J=7.6 Hz, 1H), 4.82 (ddd, J=12.1, 7.6, 5.6 Hz, 1H), 2.86-2.75 (m, 3H), 2.65-2.60 (m, 2H), 2.56 (ddd, J=17.3, 4.3, 2.7 Hz, 1H), 2.25-2.09 (m, 2H), 1.95-1.88 (m, 2H), 1.85-1.77 (m, 2H).

LCMS (m/z [M+H]⁺): 327.2

### Example 36: Synthesis of N-(2,6-dioxopiperidin-3-yl)-2-methylbenzo[b]thiophene-7-carboxamide (41)

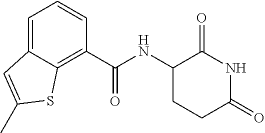 [SMILES: Cc1cc2cccc(C(=O)NC3CCC(=O)NC3=O)c2s1]

This compound was synthesized using the general procedure shown in Reaction Scheme 1 and Synthetic Conditions B, above (31% yield), and 2-methylbenzo[b]thiophene-7-carboxylic acid (20 mg) as a starting material.

¹H NMR (500 MHz, DMSO) δ 10.54 (s, 1H), 8.68 (d, J=7.7 Hz, 1H), 7.88 (dd, J=7.7, 1.3 Hz, 2H), 7.44 (t, J=7.6 Hz, 1H), 7.20-7.12 (m, 1H), 4.82 (ddd, J=11.9, 8.1, 5.5 Hz, 1H), 2.81 (ddd, J=17.5, 12.8, 5.6 Hz, 1H), 2.66-2.59 (m, 1H), 2.58 (d, J=1.1 Hz, 3H), 2.20 (qd, J=12.8, 4.6 Hz, 1H), 2.15-2.07 (m, 1H).

LCMS (m/z [M+H]⁺): 303.0

### Example 37: Synthesis of 3-bromo-N-(2,6-dioxopiperidin-3-yl)-1H-indazole-7-carboxamide (42)

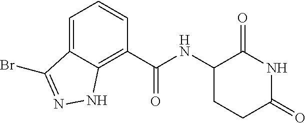 [SMILES: O=C1CCC(NC(=O)c2cccc3c(Br)n[nH]c23)C(=O)N1]

This compound was synthesized using the general procedure shown in Reaction Scheme 1 and Synthetic Conditions B, above (17% yield), and 3-bromo-1H-indazole-7-carboxylic acid (20 mg) as a starting material.

¹H NMR (500 MHz, DMSO): δ 13.46 (s, 1H), 10.92 (s, 1H), 9.07 (d, J=8.3 Hz, 1H), 8.05 (d, J=7.3 Hz, 1H), 7.82 (d, J=8.1 Hz, 1H), 7.36 (t, J=7.7 Hz, 1H), 4.87-4.79 (m, J=13.1, 8.1, 5.4 Hz, 1H), 2.90-2.77 (m, J=18.6, 13.4, 5.5 Hz, 1H), 2.63-2.56 (m, 1H), 2.22 (qd, J=12.9, 4.4 Hz, 1H), 2.08-2.02 (m, 1H).

LCMS (m/z [M+H]⁺): 351.1

### Example 38: Synthesis of N-(2,6-dioxopiperidin-3-yl)-3-(thiophen-2-yl)-1H-indazole-7-carboxamide (43)

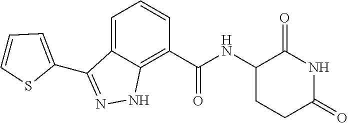 [SMILES: O=C1CCC(NC(=O)c2cccc3c(-c4cccs4)n[nH]c23)C(=O)N1]

This compound was synthesized using the general procedure shown in Reaction Scheme 1 and Synthetic Conditions B, above (32% yield), and 3-(thiophen-2-yl)-1H-indazole-7-carboxylic acid (7 mg) as a starting material.

¹H NMR (500 MHz, DMSO): δ 13.22 (s, 1H), 10.91 (s, 1H), 9.05 (d, J=7.1 Hz, 1H), 8.33 (d, J=8.1 Hz, 1H), 8.01 (d, J=7.3 Hz, 1H), 7.79 (d, J=3.1 Hz, 1H), 7.60 (d, J=4.9 Hz, 1H), 7.35 (t, J=7.7 Hz, 1H), 7.22 (dd, J=5.1, 3.6 Hz, 1H), 4.84 (ddd, J=13.2, 7.9, 5.5 Hz, 1H), 2.84 (ddd, J=18.5, 13.4, 5.5 Hz, 1H), 2.59 (dd, J=13.7, 3.3 Hz, 1H), 2.23 (qd, J=12.9, 4.4 Hz, 1H), 2.06 (dd, J=9.4, 4.6 Hz, 1H).

LCMS (m/z [M+H]⁺): 355.1

### Example 39: Synthesis of N-(2,6-dioxopiperidin-3-yl)-3-(5,6,7,8-tetrahydronaphtalen-2-yl)-1H-indazol-7-carboxamide (45)

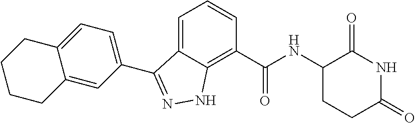 [SMILES: O=C1CCC(NC(=O)c2cccc3c(-c4ccc5c(c4)CCCC5)n[nH]c23)C(=O)N1]

This compound was synthesized using the general procedure shown in Reaction Scheme 1 and Synthetic Conditions B, above (76% yield), and 3-(5,6,7,8-tetrahydronaphtalen-2-yl)-1H-indazole-7-carboxylic acid (8 mg) as a starting material.

¹H NMR (500 MHz, DMSO) δ 13.13 (s, 1H), 10.91 (s, 1H), 9.02 (s, 1H), 8.26 (d, J=8.1 Hz, 1H), 7.98 (d, J=7.4 Hz, 1H), 7.71-7.62 (m, 2H), 7.30 (t, J=7.7 Hz, 1H), 7.21 (d, J=7.8 Hz, 1H), 4.90-4.79 (m, 1H), 2.87-2.76 (m, 5H), 2.62-2.56 (m, 1H), 2.22 (dt, J=13.3, 6.5 Hz, 1H), 2.07 (s, 1H), 1.79 (h, J=3.9, 3.5 Hz, 4H).

LCMS (m/z [M+H]⁺): 403.4

### Example 40: Synthesis of 3-(benzo[d][1,3]dioxol-5-yl)-N-(2,6-dioxopiperidin-3-yl)-1H-indazole-7-carboxamide (46)

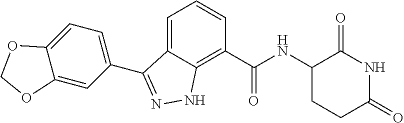 [SMILES: O=C1CCC(NC(=O)c2cccc3c(-c4ccc5c(c4)OCO5)n[nH]c23)C(=O)N1]

This compound was synthesized using the general procedure shown in Reaction Scheme 1 and Synthetic Conditions B, above (40% yield), and 3-(benzo[d][1,3]dioxol-5-yl)-1H-indazole-7-carboxylic acid (18 mg) as a starting material.

¹H NMR (500 MHz, DMSO): δ 13.13 (s, 1H), 10.91 (s, 1H), 9.02 (s, 1H), 8.24 (d, J=8.1 Hz, 1H), 7.99 (d, J=7.3 Hz, 1H), 7.54-7.42 (m, 2H), 7.30 (t, J=7.7 Hz, 1H), 7.08 (d, J=8.0 Hz, 1H), 6.10 (s, 2H), 4.91-4.79 (m, 1H), 2.84 (ddd, J=18.5, 13.4, 5.5 Hz, 1H), 2.59 (dd, J=13.8, 3.4 Hz, 1H), 2.22 (td, J=12.9, 8.9 Hz, 1H), 2.07 (s, 1H).

LCMS (m/z [M+H]⁺): 393.1

### Example 41: Synthesis of 5-bromo-N-(2,6-dioxopiperidin-3-yl)-1H-indazole-7-carboxamide (47)

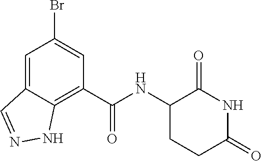 [SMILES: O=C1CCC(NC(=O)c2cc(Br)cc3cn[nH]c23)C(=O)N1]

This compound was synthesized using the general procedure shown in Reaction Scheme 1 and Synthetic Conditions B, above (14% yield), and 5-bromo-1H-indazole-7-carboxylic acid (30 mg) as a starting material.

¹H NMR (500 MHz, DMSO) δ 13.30 (s, 1H), 10.92 (s, 1H), 9.11 (d, J=6.7 Hz, 1H), 8.24 (d, J=1.5 Hz, 1H), 8.13 (d, J=17.5 Hz, 2H), 4.89-4.79 (m, 1H), 2.88-2.78 (m, J=18.5, 13.3, 5.5 Hz, 1H), 2.62-2.54 (m, J=13.7, 3.5 Hz, 1H), 2.18 (qd, J=12.8, 4.2 Hz, 1H), 2.09-1.94 (m, 1H).

LCMS (m/z [M+H]⁺): 351.1

### Example 42: Synthesis of 6-amino-N-(2,6-dioxopiperidin-3-yl)-1H-indazole-7-carboxamide (48)

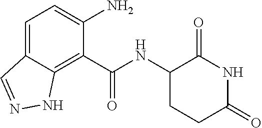 [SMILES: Nc1ccc2cn[nH]c2c1C(=O)NC1CCC(=O)NC1=O]

This compound was synthesized using the general procedure shown in Reaction Scheme 1 and Synthetic Conditions B, above (20% yield), and 6-amino-1H-indazole-7-carboxylic acid (30 mg) as a starting material.

¹H NMR (500 MHz, DMSO) δ 13.57 (s, 1H), 10.88 (s, 1H), 10.00 (d, J=6.9 Hz, 1H), 8.25 (s, 1H), 7.60 (d, J=9.0 Hz, 1H), 6.64 (d, J=9.0 Hz, 1H), 4.90-4.78 (m, J=12.5, 6.3 Hz, 1H), 2.91-2.73 (m, 1H), 2.59 (s, 1H), 2.30-2.19 (m, 1H), 2.11-1.96 (m, J=23.7, 11.1 Hz, 1H).

LCMS (m/z [M+H]⁺): 288.2

### Example 43: Synthesis of N-(2,6-dioxopiperidin-3-yl)benzo[d]isothiazole-7-carboxamide (50)

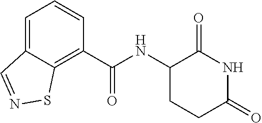 [SMILES: O=C1CCC(NC(=O)c2cccc3cnsc23)C(=O)N1]

This compound was synthesized using the general procedure shown in Reaction Scheme 1 and Synthetic Conditions B, above (48% yield), and benzo[d]isothiazole-7-carboxylic acid (20 mg) as a starting material.

¹H NMR (500 MHz, DMSO) δ 10.93 (s, 1H), 9.32 (d, J=8.3 Hz, 1H), 9.17 (s, 1H), 8.44 (dd, J=7.8, 0.9 Hz, 1H), 8.33 (dd, J=7.5, 0.9 Hz, 1H), 7.70 (t, J=7.6 Hz, 1H), 4.89 (ddd, J=13.3, 8.2, 5.4 Hz, 1H), 2.84 (ddd, J=17.4, 13.4, 5.5 Hz, 1H), 2.59 (dt, J=17.2, 3.9 Hz, 1H), 2.21 (qd, J=13.0, 4.5 Hz, 1H), 2.09-2.00 (m, 1H).

LCMS (m/z [M+H]⁺): 290.3

### Example 44: Synthesis of N-(2,6-dioxopiperidin-3-yl)-2-methylbenzo[d]oxazole-4-carboxamide (51)

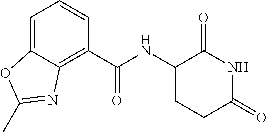 [SMILES: Cc1nc2c(C(=O)NC3CCC(=O)NC3=O)cccc2o1]

This compound was synthesized using the general procedure shown in Reaction Scheme 1 and Synthetic Conditions B, above (35% yield), and 2-methylbenzo[d]oxazole-4-carboxylic acid (20 mg) as a starting material.

¹H NMR (500 MHz, DMSO) δ 10.90 (s, 1H), 8.62 (d, J=8.0 Hz, 1H), 7.84 (dd, J=7.9, 1.1 Hz, 1H), 7.74 (dd, J=7.8, 1.2 Hz, 1H), 7.44 (t, J=7.8 Hz, 1H), 4.86-4.79 (m, 1H), 2.82 (ddd, J=17.3, 13.4, 5.6 Hz, 1H), 2.67 (s, 3H), 2.56 (ddd, J=17.3, 4.4, 2.7 Hz, 1H), 2.19 (qd, J=12.9, 4.5 Hz, 1H), 2.12-2.04 (m, 1H).

LCMS (m/z [M+H]⁺): 288.0

### Example 45: Synthesis of N-(2,6-dioxopiperidin-3-yl)-2-methylbenzo[d]oxazole-7-carboxamide (52)

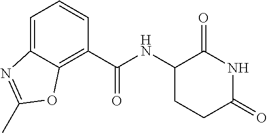 [SMILES: Cc1nc2cccc(C(=O)NC3CCC(=O)NC3=O)c2o1]

This compound was synthesized using the general procedure shown in Reaction Scheme 1 and Synthetic Conditions B, above (47% yield), and 2-methylbenzo[d]oxazole-7-carboxylic acid (20 mg) as a starting material.

¹H NMR (500 MHz, DMSO) δ 10.95 (s, 1H), 9.30 (d, J=7.2 Hz, 1H), 7.94 (ddd, J=16.3, 7.9, 1.0 Hz, 2H), 7.50 (t, J=8.0 Hz, 1H), 4.89 (ddd, J=12.6, 7.2, 5.3 Hz, 1H), 2.87-2.77 (m, 1H), 2.72 (s, 3H), 2.56 (ddd, J=17.6, 4.5, 2.5 Hz, 1H), 2.24 (dtd, J=13.1, 5.5, 2.4 Hz, 1H), 2.18-2.08 (m, 1H).

LCMS (m/z [M+H]⁺): 287.8

### Example 46: Synthesis of N-(2,6-dioxopiperidin-3-yl)-2-methylbenzo[d]thiazole-7-carboxamide (53)

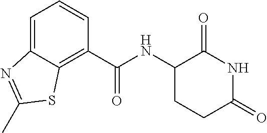 [SMILES: Cc1nc2cccc(C(=O)NC3CCC(=O)NC3=O)c2s1]

This compound was synthesized using the general procedure shown in Reaction Scheme 1 and Synthetic Conditions B, above (32% yield), and 2-methylbenzo[d]thiazole-7-carboxylic acid (20 mg) as a starting material.

¹H NMR (500 MHz, DMSO) δ 10.90 (s, 1H), 9.13 (d, J=8.3 Hz, 1H), 8.10 (dd, J=8.0, 0.9 Hz, 1H), 8.06 (dd, J=7.7, 1.0 Hz, 1H), 7.63 (t, J=7.8 Hz, 1H), 4.86 (ddd, J=12.5, 8.2, 5.4 Hz, 1H), 2.87-2.77 (m, 4H), 2.57 (ddd, J=17.3, 4.4, 2.8 Hz, 1H), 2.18 (qd, J=13.0, 4.5 Hz, 1H), 2.02 (dtd, J=13.2, 5.5, 2.8 Hz, 1H).

LCMS (m/z [M+H]⁺): 304.0

### Example 47: Synthesis of N-(2,6-dioxopiperidin-3-yl)thiazolo[5,4-b]pyridine-7-carboxamide (54)

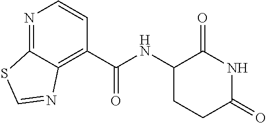 [SMILES: O=C1CCC(NC(=O)c2ccnc3scnc23)C(=O)N1]

This compound was synthesized using the general procedure shown in Reaction Scheme 1 and Synthetic Conditions B, above (56% yield), and thiazolo[5,4-b]pyridine-7-carboxylic acid (20 mg) as a starting material.

¹H NMR (500 MHz, DMSO) δ 10.98 (s, 1H), 9.93 (d, J=7.2 Hz, 1H), 9.86-9.80 (m, 1H), 8.89 (d, J=4.8 Hz, 1H), 8.08 (d, J=4.8 Hz, 1H), 4.93 (ddd, J=12.6, 7.2, 5.4 Hz, 1H), 2.83 (ddd, J=17.5, 13.5, 5.6 Hz, 1H), 2.53-2.51 (m, 1H), 2.25 (dtd, J=13.1, 5.5, 2.4 Hz, 1H), 2.20-2.10 (m, 1H).

LCMS (m/z [M+H]⁺): 290.9

### Example 48: Synthesis of N-(2,6-dioxopiperidin-3-yl)-1H-benzo[d][1,2,3]triazole-4-carboxamide (55)

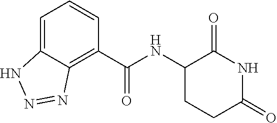 [SMILES: O=C1CCC(NC(=O)c2cccc3[nH]nnc23)C(=O)N1]

This compound was synthesized using the general procedure shown in Reaction Scheme 1 and Synthetic Conditions C, above (76% yield), and 1H-benzo[d][1,2,3]triazole-4-carboxylic acid (20 mg) as a starting material.

¹H NMR (500 MHz, DMSO, 353K) S 15.65 (s, 1H), 10.61 (s, 1H), 9.26 (s, 1H), 8.18-8.00 (m, 2H), 7.58 (t, J=7.7 Hz, 1H), 4.89 (dt, J=12.8, 6.6 Hz, 1H), 2.83 (ddd, J=17.2, 12.8, 5.8 Hz, 1H), 2.63 (dt, J=17.4, 3.8 Hz, 1H), 2.21 (qd, J=13.2, 12.7, 5.4 Hz, 2H).

LCMS (m/z [M+H]⁺): 274.1

### Example 49: Synthesis of N-(2,6-dioxopiperidin-3-yl)-6-nitro-1H-benzo[d][1,2,3]triazole-4-carboxamide (57)

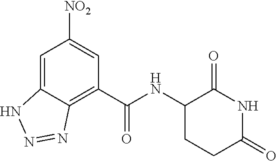 [SMILES: O=C1CCC(NC(=O)c2cc([N+](=O)[O-])cc3[nH]nnc23)C(=O)N1]

This compound was synthesized using the general procedure shown in Reaction Scheme 1 and Synthetic Conditions C, above (48% yield), and 6-nitro-1H-benzo[d][1,2,3]triazole-4-carboxylic acid (5 mg) as a starting material.

¹H NMR (500 MHz, DMSO) δ 10.93 (s, 1H), 9.32 (d, J=7.2 Hz, 1H), 8.81 (d, J=2.2 Hz, 1H), 8.48 (d, J=2.2 Hz, 1H), 4.78-4.67 (m, J=12.6, 7.0, 5.5 Hz, 1H), 2.82 (ddd, J=17.5, 13.6, 5.6 Hz, 1H), 2.53 (s, 1H), 2.26-2.08 (m, 2H).

### Example 50: Synthesis of N-(2,6-dioxopiperidin-3-yl)benzo[d][1,2,3]thiadiazole-7-carboxamide (58)

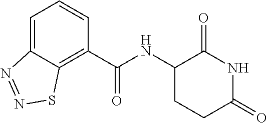 [SMILES: O=C1CCC(NC(=O)c2cccc3nnsc23)C(=O)N1]

This compound was synthesized using the general procedure shown in Reaction Scheme 1 and Synthetic Conditions B, above (62% yield), and benzo[d][1,2,3]thiadiazole-7-carboxylic acid (10 mg) as a starting material.

¹H NMR (500 MHz, DMSO) δ 10.95 (s, 1H), 9.51 (d, J=8.2 Hz, 1H), 9.01-8.89 (m, 1H), 8.53 (dd, J=7.4, 0.8 Hz, 1H), 7.97 (dd, J=8.2, 7.4 Hz, 1H), 4.96-4.84 (m, 1H), 2.84 (ddd, J=17.4, 13.4, 5.5 Hz, 1H), 2.59 (ddd, J=17.3, 4.5, 2.7 Hz, 1H), 2.21 (qd, J=13.0, 4.5 Hz, 1H), 2.05 (dtd, J=13.0, 5.3, 2.7 Hz, 1H).

LCMS (m/z [M+H]⁺): 291.1

### Example 51: Synthesis of N-(2,6-dioxopiperidin-3-yl)-2-methyl-1H-thieno[2,3-d]imidazole-6-carboxamide (59)

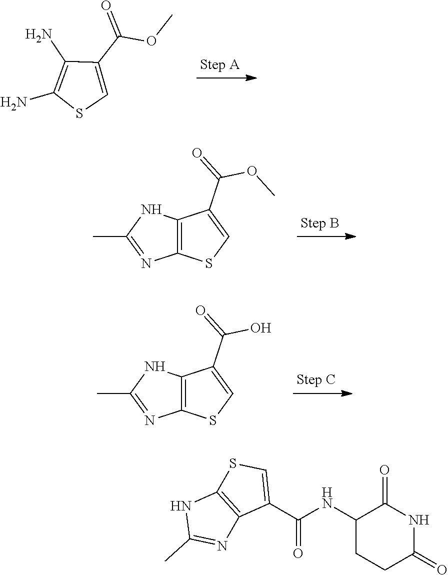

Step A: A mixture of methyl 4,5-diaminothiophene-3-carboxylate (400 mg, 2.04 mmol) in dioxane (3 mL), triethyl orthoacetate (3 mL) and PTSA (102 mg, 0.40 mmol) was heated to reflux for 16 h, the reaction mixture was concentrated under reduced pressure and the crude material was purified by flash column chromatography to give methyl 2-methyl-1H-thieno[2,3-d]imidazole-6-carboxylate 200 mg (50% yield).

Step B: To a stirred solution of methyl 2-methyl-1H-thieno[2,3-d]imidazole-6-carboxylate (0.13 g, 1.02 mmol) in methanol (0.5 mL) and THE (2 mL) was added NaOH (27 mg, 0.68 mmol) in water (0.5 mL) and the resulting solution was stirred at RT for 16 h. The reaction mixture was diluted with water and washed with ethyl acetate. The aqueous part was acidified with 6N HCl to pH˜5 and the resulting precipitate was filtered, washed with water and purified by HPLC to give 2-methyl-1H-thieno[2,3-d]imidazole-6-carboxylic acid 70 mg (37%).

Step C: N-(2,6-dioxopiperidin-3-yl)-2-methyl-1H-thieno[2,3-d]imidazole-6-carboxamide was synthesized using the general procedure shown in Reaction Scheme 1 and Synthetic Conditions B, above (17% yield), and 2-methyl-3H-thieno[2,3-d]imidazole-6-carboxylic acid (20 mg) as a starting material.

¹H NMR (500 MHz, DMSO): δ 12.53 (s, 1H), 10.87 (s, 1H), 8.70 (d, J=8.0 Hz, 1H), 7.82 (s, 1H), 4.78-4.67 (m, 1H), 2.81 (ddd, J=17.4, 13.3, 5.5 Hz, 1H), 2.56 (ddd, J=17.1, 4.1, 2.9 Hz, 1H), 2.43 (s, 3H), 2.16 (qd, J=12.9, 4.5 Hz, 1H), 2.04-1.96 (m, 1H).

LCMS (m/z [M+H]⁺): 293.0

### Example 52: Synthesis of N-(2,6-dioxopiperidin-3-yl)-1H-thieno[2,3-d]imidazole-6-carboxamide (60)

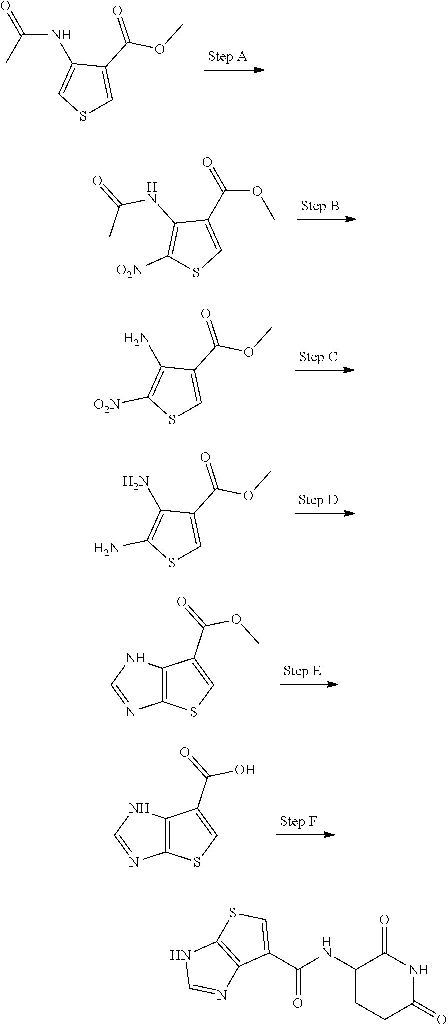

Step A: A Solution of methyl 4-acetamidothiophene-3-carboxylate (3 g, 12.3 mmol) in acetic anhydride (40 mL) was cooled at −15° C. To it a precooled solution (at −15° C.) of concentrated nitric acid (6 mL) in 30 mL acetic anhydride was added drop wise very slowly with stirring. After 30 min the reaction mixture was poured into crushed ice and the resulting light yellow coloured solid was filtered. The solid was thoroughly washed with water and diethyl ether to give 2.4 g (81%) of methyl 4-acetamido-5-nitrothiophene-3-carboxylate.

Step B: To a stirred solution of methyl 4-acetamido-5-nitrothiophene-3-carboxylate (2 g, 8.19 mmol) in 4N HCl-dioxane (20 mL), methanol (10 mL) was added and the resulting solution was heated at 100° C. for 16 h. After cooling, dioxane was removed under reduced pressure. The residue was diluted with water and extracted with ethyl acetate. The organic layer was washed with saturated sodium bicarbonate and brine and dried over Na₂SO₄. After concentration under reduced pressure, the crude methyl 4-amino-5-nitrothiophene-3-carboxylate 850 mg (51%) was used in the next step without further purification.

Step C: To a stirred solution of methyl 4-amino-5-nitrothiophene-3-carboxylate (1 g, 4.95 mmol) in a mixture of dioxane-HCl (10 mL) and methanol (10 mL), SnCl₂ was added and the resulting solution was stirred at RT for 2 h. The reaction mixture was then poured on to a precooled solution of ammonium hydroxide and extracted with ethyl acetate. The organic layer was dried over anhydrous sodium sulfate, filtered and dried under reduced pressure. The crude methyl 4,5-diamino-thiophene-3-carboxylate 700 mg (82%) was used in the next step without further purification.

Step D: To a stirred solution of methyl 4,5-diaminothiophene-3-carboxylate (650 mg, 3.78 mmol) in a mixture of trimethyl orthoformate (2.5 mL) and toluene (2.5 mL), a catalytic amount of PTSA (189 mg, 0.75 mmol) was added and the resulting solution was heated at 110° C. for 2 h. After that the volatiles were removed under reduced pressure, the crude material was purified by flash column chromatography to give 350 mg (50%) of methyl 1H-thieno[2,3-d]imidazole-6-carboxylate.

Step E: To a stirred solution of methyl 1H-thieno[2,3-d]imidazole-6-carboxylate (400 mg, 2.2 mmol mmol) in methanol (3 mL) and THE (3 mL), NaOH (439 mg, 10.9 mmol) dissolved in water (1 mL) was added and the resulting solution was stirred for 16 h. The reaction mixture was diluted with water and washed with ethyl acetate. The aqueous part was acidified with 6N HCl to pH˜5 and the resulting brown coloured precipitate was filtered, washed with water and diethyl ether to obtain 1H-thieno[2,3-d]imidazole-6-carboxylic acid 230 mg (62%).

Step F: N-(2,6-dioxopiperidin-3-yl)-1H-thieno[2,3-d]imidazole-6-carboxamide was synthesized using the general procedure shown in Reaction Scheme 1 and Synthetic Conditions B, above (40% yield), and 1H-thieno[2,3-d]imidazole-6-carboxylic acid (20 mg) as a starting material.

¹H NMR (500 MHz, DMSO) δ 12.79 (s, 1H), 10.88 (s, 1H), 8.74 (d, J=8.2 Hz, 1H), 7.99 (d, J=1.3 Hz, 1H), 7.90 (s, 1H), 4.74 (ddd, J=13.3, 8.1, 5.3 Hz, 1H), 2.81 (ddd, J=17.2, 13.3, 5.5 Hz, 1H), 2.57 (dt, J=18.0, 4.1 Hz, 1H), 2.16 (qd, J=12.9, 4.5 Hz, 1H), 2.01 (dtd, J=13.1, 5.4, 2.8 Hz, 1H).

LCMS (m/z [M+H]⁺): 279.0

### Example 53: Synthesis of N-(2,6-dioxopiperidin-3-yl)-2,5,6-trimethyl-4H-thieno[3,2-b]pyrrole-3-carboxamide (61)

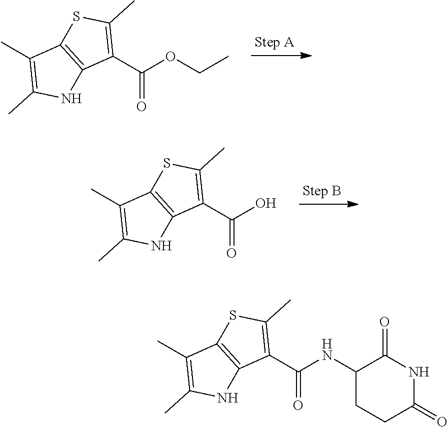 [SMILES: CCOC(=O)c1c(C)sc2c(C)c(C)[nH]c12 ; Cc1[nH]c2c(C(=O)O)c(C)sc2c1C ; Cc1[nH]c2c(C(=O)NC3CCC(=O)NC3=O)c(C)sc2c1C]

Step A: To a solution of ethyl 2,5,6-trimethyl-4H-thieno[3,2-b]-pyrrole-3-carboxylate (10.0 mg, 0.042 mmol, 1.000 eq) in a mixture of H₂O (1.0 mL), THE (1.0 mL) and MeOH (1.0 mL) was added 1M LiOH (2.0 mL, 2.000 mmol, 17.702 eq). The reaction was stirred for 24 h at rt. After this time, to a mixture was added 1M HCl (2.0 mL, 2.000 mmol, 17.702 eq) to neutralize pH. The crude was concentrated in vacuo and used to the next step without further purification.

Step B: N-(2,6-dioxopiperidin-3-yl)-2,5,6-trimethyl-4H-thieno[3,2-b]pyrrole-3-carboxamide was synthesized using the general procedure shown in Reaction Scheme 1 and Synthetic Conditions B, above (23% yield), and 2,5,6-trimethyl-4H-thieno[3,2-b]pyrrole-3-carboxylic acid (8.8 mg) as a starting material. 1H NMR (500 MHz, DMSO) δ 10.87 (s, 1H), 10.45 (s, 1H), 7.94 (d, J=8.2 Hz, 1H), 4.76 (ddd, J=12.3, 8.2, 5.4 Hz, 1H), 2.80 (ddd, J=17.3, 13.4, 5.6 Hz, 1H), 2.63 (s, 3H), 2.59-2.52 (m, 1H), 2.22 (s, 3H), 2.16 (qd, J=13.0, 4.5 Hz, 1H), 2.05 (qd, J=4.8, 2.3 Hz, 1H), 2.02 (s, 3H).

LCMS (m/z [M+H]⁺): 319.8

### Example 54: Synthesis of N-(2,6-dioxopiperidin-3-yl)thieno[3,4-b]thiophene-2-carboxamide (62)

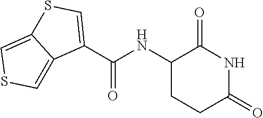 [SMILES: O=C1CCC(NC(=O)c2csc3cscc23)C(=O)N1]

This compound was synthesized using the general procedure shown in Reaction Scheme 1 and Synthetic Conditions B, above (50% yield), and thieno[3,4-b]thiophene-2-carboxylic acid (10 mg) as a starting material.

¹H NMR (500 MHz, DMSO) δ 10.89 (s, 1H), 8.94 (d, J=8.3 Hz, 1H), 7.97 (d, J=2.7 Hz, 1H), 7.76 (s, 1H), 7.71 (dd, J=2.7, 0.8 Hz, 1H), 4.78-4.71 (m, 1H), 2.85-2.76 (m, 1H), 2.59-2.52 (m, 1H), 2.12 (qd, J=12.9, 4.5 Hz, 1H), 2.00 (dtd, J=12.9, 5.4, 2.8 Hz, 1H).

LCMS (m/z [M+H]⁺): 294.5

### Example 55: Synthesis of 2-(3-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)amino)-3-oxopropyl)-N-(2,6-dioxopiperidin-3-yl)-1H-benzo[d]imidazole-7-carboxamide (63)

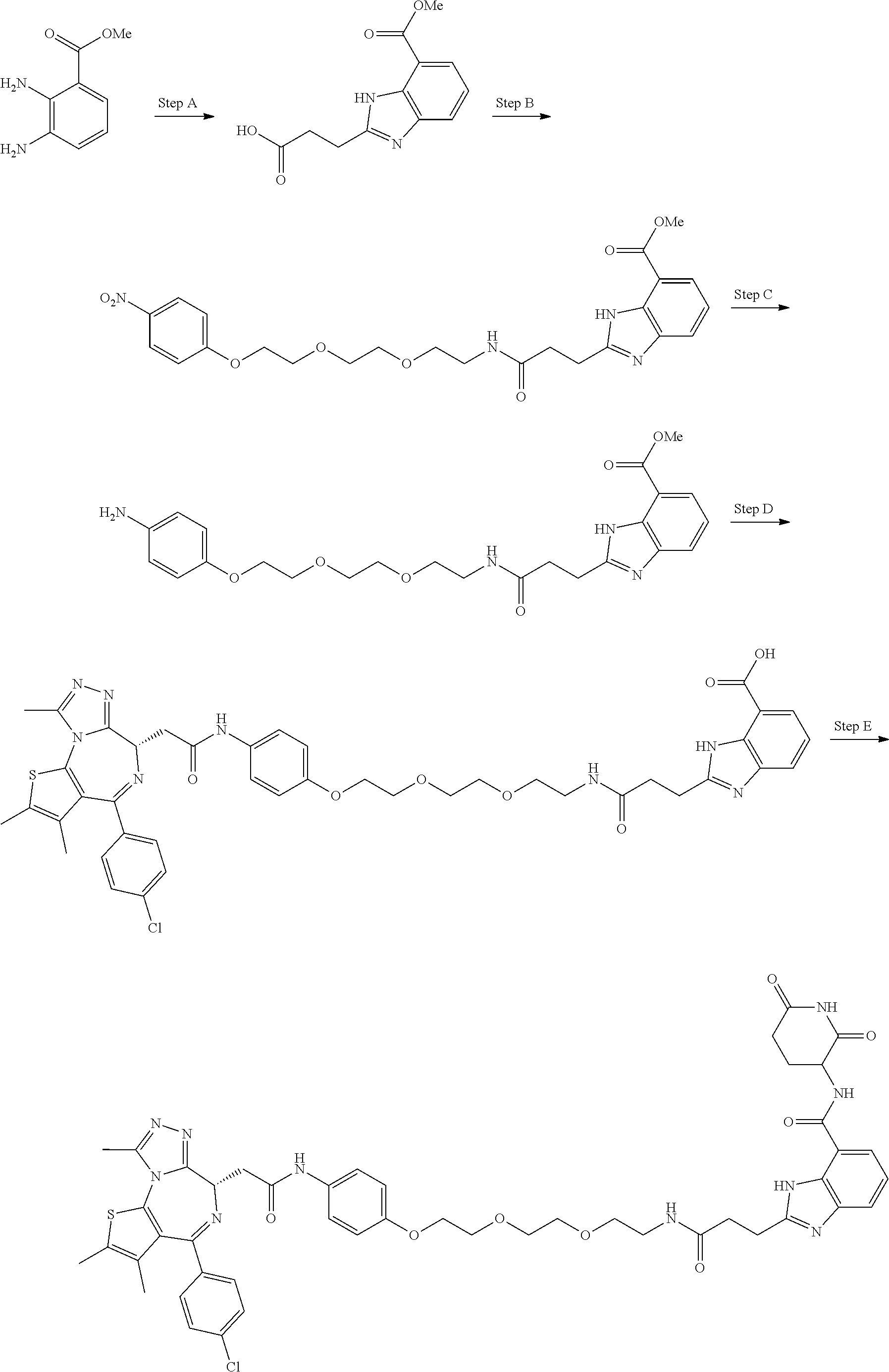 [SMILES: COC(=O)c1cccc(N)c1N ; COC(=O)c1cccc2nc(CCC(=O)O)[nH]c12 ; COC(=O)c1cccc2nc(CCC(=O)NCCOCCOCCOc3ccc([N+](=O)[O-])cc3)[nH]c12 ; COC(=O)c1cccc2nc(CCC(=O)NCCOCCOCCOc3ccc(N)cc3)[nH]c12 ; Cc1sc2c(c1C)C(c1ccc(Cl)cc1)=N[C@@H](CC(=O)Nc1ccc(OCCOCCOCCNC(=O)CCc3nc4cccc(C(=O)O)c4[nH]3)cc1)c1nnc(C)n1-2 ; Cc1sc2c(c1C)C(c1ccc(Cl)cc1)=N[C@@H](CC(=O)Nc1ccc(OCCOCCOCCNC(=O)CCc3nc4cccc(C(=O)NC5CCC(=O)NC5=O)c4[nH]3)cc1)c1nnc(C)n1-2]

Step A: A mixture of methyl 2,3-diaminobenzoate (2 g, 12.05 mmol) and succinic anhydride (1.2 g, 12.05 mmol) in acetic acid (70 mL) was heated at 80° C. for 16 h. After completion of reaction, acetic acid was removed under reduced pressure. The crude product was triturated with water (10 mL) and filtered, the solid was washed with cold water (5 mL) and dried in vacuum to provide 3-(7-(methoxycarbonyl)-1H-benzo[d]imidazol-2-yl)propanoic acid 2.5 g (83%).

Step B: To a solution 2-(2-(2-(4-nitrophenoxy)ethoxy)ethoxy)ethanamine (77 mg, 0.251 mmol, 1 eq), 3-(7-(methoxycarbonyl)-1H-benzo[d]imidazol-2-yl)propanoic acid (74.8 mg, 0.301 mmol, 1.2 eq), DMAP (3.1 mg, 0.025 mmol, 0.1 equiv) and HATU (114.5 mg, 0.301 mmol, 1.2 eq) in DMF (13 mL) was added DIPEA (0.175 mL, 1.0 mmol, 4 eq). The reaction mixture was stirred at RT for 2 h. After evaporation of the solvent, the crude product was purified by HPLC to provide methyl 2-(3-((2-(2-(2-(4-nitrophenoxy)ethoxy)ethoxy)ethyl)amino)-3-oxopropyl)-1H-benzo[d]imidazole-7-carboxylate 87 mg (69%).

Step C: The methyl 2-(3-((2-(2-(2-(4-nitrophenoxy)ethoxy)ethoxy)ethyl)amino)-3-oxopropyl)-1H-benzo[d]imidazole-7-carboxylate (85 mg, 0.170 mmol, 1 eq) was dissolved in 20 mL of EtOH and 10 mL of water. Then NH₄Cl (2.27 g, 250 eq) was added followed by Fe powder (663 mg, 70 eq) and the flask was immediately closed with septum. The slurry was stirred at 40° C. for 3 h. The mixture was diluted with water and filtered on Celite and the solid residue was washed with DCM. The filtrates were extracted with DCM, dried over Na₂SO₄ and evaporated yielding methyl 2-(3-((2-(2-(2-(4-aminophenoxy)ethoxy)ethoxy)ethyl)amino)-3-oxopropyl)-1H-benzo[d]imidazole-7-carboxylate 77 mg (97%).

Step D: To a solution of methyl 2-(3-((2-(2-(2-(4-aminophenoxy)ethoxy)ethoxy)ethyl)amino)-3-oxopropyl)-1H-benzo[d]imidazole-7-carboxylate (75 mg, 0.159 mmol, 1.04 eq), (S)-[4-(4-chlorophenyl)-2,3,9-trimethyl-6H-thieno[3,2-f][1,2,4]triazolo[4,3-a][1,4]diazepin-6-yl]acetic acid (61.5 mg, 0.15 mmol, 1 eq), HATU (72.7 mg, 0.191 mmol, 1.2 eq) and DMAP (1.9 mg, 0.016 mmol, 0.1 eq) in DMF (8 mL) was added DIPEA (0.111 mL, 0.638 mmol, 4 eq) and the reaction mixture was stirred at RT for 3 h. DMF was removed under reduced pressure and the residue was redissolved in methanol (8 mL). 1M lithium hydroxide in water (8 mL) was added and the reaction mixture was stirred at RT for 2 h. The mixture was neutralized with 1M HCl, concentrated under reduced pressure and purified by HPLC to give (S)-2-(3-((2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)amino)-3-oxopropyl)-1H-benzo[d]imidazole-7-carboxylic acid (40 mg, 30%).

Step E: (S)-2-(3-((2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)amino)-3-oxopropyl)-1H-benzo[d]imidazole-7-carboxylic acid (21.5 mg, 0.026 mmol, 1 eq), 3-aminopiperidine-2,6-dione hydrochloride (12.6 mg, 0.77 mmol, 3 eq), HATU (29.2 mg, 0.077 mmol, 3 eq) and DMAP (0.3 mg, 0.003 mmol, 0.1 eq) were dissolved in DMF (2 mL). DIPEA (0.036 mL, 0.205 mmol, 8 eq) was added and the reaction mixture was stirred at RT for 2 h. The solvent was removed under reduced pressure and the residue was purified by preparative HPLC to give 2-(3-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)amino)-3-oxopropyl)-N-(2,6-dioxopiperidin-3-yl)-1H-benzo[d]imidazole-7-carboxamide (14.6 mg, 60%).

¹H NMR (500 MHz, DMSO) δ 12.69 (s, 1H), 10.91 (s, 1H), 10.35 (d, J=6.7 Hz, 1H), 10.15 (s, 1H), 7.97 (s, 1H), 7.81 (d, J=7.5 Hz, 1H), 7.64 (d, J=7.7 Hz, 1H), 7.55-7.50 (m, 2H), 7.48 (d, J=8.8 Hz, 2H), 7.42 (d, J=8.6 Hz, 2H), 7.27 (t, J=7.7 Hz, 1H), 6.89 (d, J=9.1 Hz, 2H), 4.86 (d, J=6.7 Hz, 1H), 4.59 (t, J=7.1 Hz, 1H), 4.07-4.00 (m, 2H), 3.74-3.66 (m, 2H), 3.54 (d, J=4.8 Hz, 2H), 3.49 (d, J=4.8 Hz, 2H), 3.46 (d, J=7.1 Hz, 2H), 3.39 (t, J=5.8 Hz, 2H), 3.20 (dd, J=11.4, 5.6 Hz, 2H), 3.11 (t, J=7.4 Hz, 2H), 2.88-2.77 (m, 1H), 2.71 (t, J=7.3 Hz, 2H), 2.60 (s, 2H), 2.57 (d, J=18.5 Hz, 1H), 2.42 (d, J=0.6 Hz, 3H), 2.24 (s, 1H), 2.20-2.09 (m, 1H), 1.63 (d, J=0.6 Hz, 3H).

LCMS (m/z [M+H]⁺): 949.9

### Example 56: Synthesis of 2-(3-((8-(2-((S)-4-(4-chlorophenyl)-2,3,9-trimethyl-6H-thieno[3,2-f][1,2,4]triazolo[4,3-a][1,4]diazepin-6-yl)acetamido)octyl)amino)-3-oxopropyl)-N-(2,6-dioxopiperidin-3-yl)-1H-benzo[d]imidazole-7-carboxamide (64)

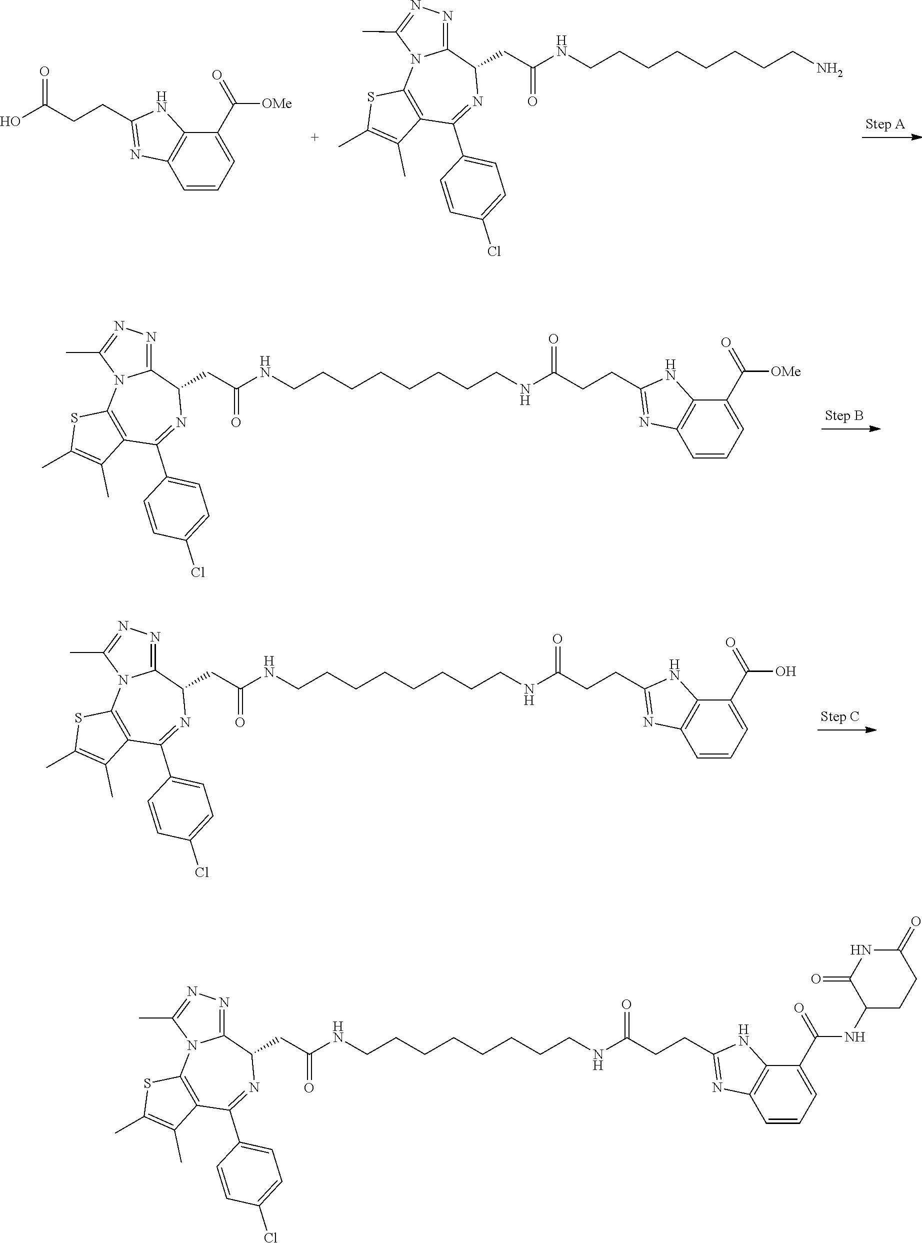 [SMILES: COC(=O)c1cccc2nc(CCC(=O)O)[nH]c12 ; Cc1sc2c(c1C)C(c1ccc(Cl)cc1)=N[C@@H](CC(=O)NCCCCCCCCN)c1nnc(C)n1-2 ; COC(=O)c1cccc2nc(CCC(=O)NCCCCCCCCNC(=O)C[C@@H]3N=C(c4ccc(Cl)cc4)c4c(sc(C)c4C)-n4c(C)nnc43)[nH]c12 ; Cc1sc2c(c1C)C(c1ccc(Cl)cc1)=N[C@@H](CC(=O)NCCCCCCCCNC(=O)CCc1nc3cccc(C(=O)O)c3[nH]1)c1nnc(C)n1-2 ; Cc1sc2c(c1C)C(c1ccc(Cl)cc1)=N[C@@H](CC(=O)NCCCCCCCCNC(=O)CCc1nc3cccc(C(=O)NC4CCC(=O)NC4=O)c3[nH]1)c1nnc(C)n1-2]

Step A: To a solution of (S)—N-(8-aminooctyl)-2-(4-(4-chlorophenyl)-2,3,9-trimethyl-6H-thieno[3,2-f][1,2,4]triazolo[4,3-a][1,4]diazepin-6-yl)acetamide (59.2 mg, 0.105 mmol, 1 eq), 3-(7-(methoxycarbonyl)-1H-benzo[d]imidazol-2-yl)propanoic acid (31.3 mg, 0.126 mmol, 1.2 eq), HATU (47.9 mg, 0.126 mmol, 1.2 eq) and DMAP (1.3 mg, 0.011 mmol, 0.1 eq) in DMF (5 mL) was added DIPEA (0.110 mL, 0.630 mmol, 6 eq). The reaction mixture was stirrer at RT for 18 h, solvent was removed under reduced pressure and the residue was purified by flash column chromatography to provide methyl (S)-2-(3-((8-(2-(4-(4-chlorophenyl)-2,3,9-trimethyl-6H-thieno[3,2-f][1,2,4]triazolo[4,3-a][1,4]diazepin-6-yl)acetamido)octyl)amino)-3-oxopropyl)-1H-benzo[d]imidazole-7-carboxylate 79.5 mg (>99%).

Step B: To a solution of methyl (S)-2-(3-((8-(2-(4-(4-chlorophenyl)-2,3,9-trimethyl-6H-thieno[3,2-f][1,2,4]triazolo[4,3-a][1,4]diazepin-6-yl)acetamido)octyl)amino)-3-oxopropyl)-1H-benzo[d]imidazole-7-carboxylate (79.5 mg, 0.105 mmol, 1 eq) in THE (2.5 mL), methanol (0.5 mL) and water (0.9 mL) was added lithium hydroxide (80 mg, 3.34 mmol) and the reaction mixture was stirred at RT for 18 h. The solution was acidified with 1M HCl and extracted with ethyl acetate. Organic phases were dried over Na₂SO₄ and concentrated under reduced pressure to give (S)-2-(3-((8-(2-(4-(4-chlorophenyl)-2,3,9-trimethyl-6H-thieno[3,2-f][1,2,4]triazolo[4,3-a][1,4]diazepin-6-yl)acetamido)octyl)amino)-3-oxopropyl)-1H-benzo[d]imidazole-7-carboxylic acid (75 mg, 96%).

Step C: (S)-2-(3-((8-(2-(4-(4-chlorophenyl)-2,3,9-trimethyl-6H-thieno[3,2-f][1,2,4]triazolo[4,3-a][1,4]diazepin-6-yl)acetamido)octyl)amino)-3-oxopropyl)-1H-benzo[d]imidazole-7-carboxylic acid (70 mg, 0.094 mmol, 1 eq), 3-aminopiperidine-2,6-dione hydrochloride (18.6 mg, 0.113 mmol, 1.2 eq), HATU (43 mg, 0.113 mmol, 1.2 eq) and DMAP (0.2 mg, 0.009 mmol, 0.1 eq) were dissolved in DMF (3.6 mL). DIPEA (0.049 mL, 0.283 mmol, 3 eq) was added and the reaction mixture was stirred at RT for 18 h. The solvent was removed under reduced pressure and the residue was purified by preparative HPLC to give 2-(3-((8-(2-((S)-4-(4-chlorophenyl)-2,3,9-trimethyl-6H-thieno[3,2-f][1,2,4]triazolo[4,3-a][1,4]diazepin-6-yl)acetamido)octyl)amino)-3-oxopropyl)-N-(2,6-dioxopiperidin-3-yl)-1H-benzo[d]imidazole-7-carboxamide 31 mg (26%).

¹H NMR (500 MHz, DMSO) δ 12.70 (s, 1H), 10.93 (s, 1H), 10.37 (s, 1H), 8.14 (t, J=5.6 Hz, 1H), 7.83 (s, 1H), 7.82 (s, 1H), 7.66 (s, 1H), 7.47 (d, J=8.8 Hz, 2H), 7.41 (dd, J=10.2, 8.4 Hz, 2H), 7.29 (s, 1H), 4.85 (d, J=5.2 Hz, 1H), 4.55-4.46 (m, 1H), 3.28-3.15 (m, 2H), 3.15-3.05 (m, 4H), 3.05-2.95 (m, 2H), 2.83 (ddd, J=17.5, 13.3, 5.6 Hz, 1H), 2.68 (t, J=7.4 Hz, 2H), 2.59 (s, 3H), 2.55 (dd, J=10.8, 3.7 Hz, 1H), 2.40 (d, J=0.5 Hz, 3H), 2.36 (dt, J=14.1, 6.1 Hz, 1H), 2.32-2.24 (m, 1H), 1.62 (d, J=0.5 Hz, 3H), 1.45-1.36 (m, 2H), 1.36-1.28 (m, 2H), 1.23 (s, 3H), 1.16 (s, 5H).

LCMS (m/z [M+H]⁺): 852.9

### Example 57: Synthesis of 1-(2-((8-(2-((S)-4-(4-chlorophenyl)-2,3,9-trimethyl-6H-thieno[3,2-f][1,2,4]triazolo[4,3-a][1,4]diazepin-6-yl)acetamido)octyl)amino)-2-oxoethyl)-N-(2,6-dioxopiperidin-3-yl)-2-methyl-1H-benzo[d]imidazole-4-carboxamide (65)

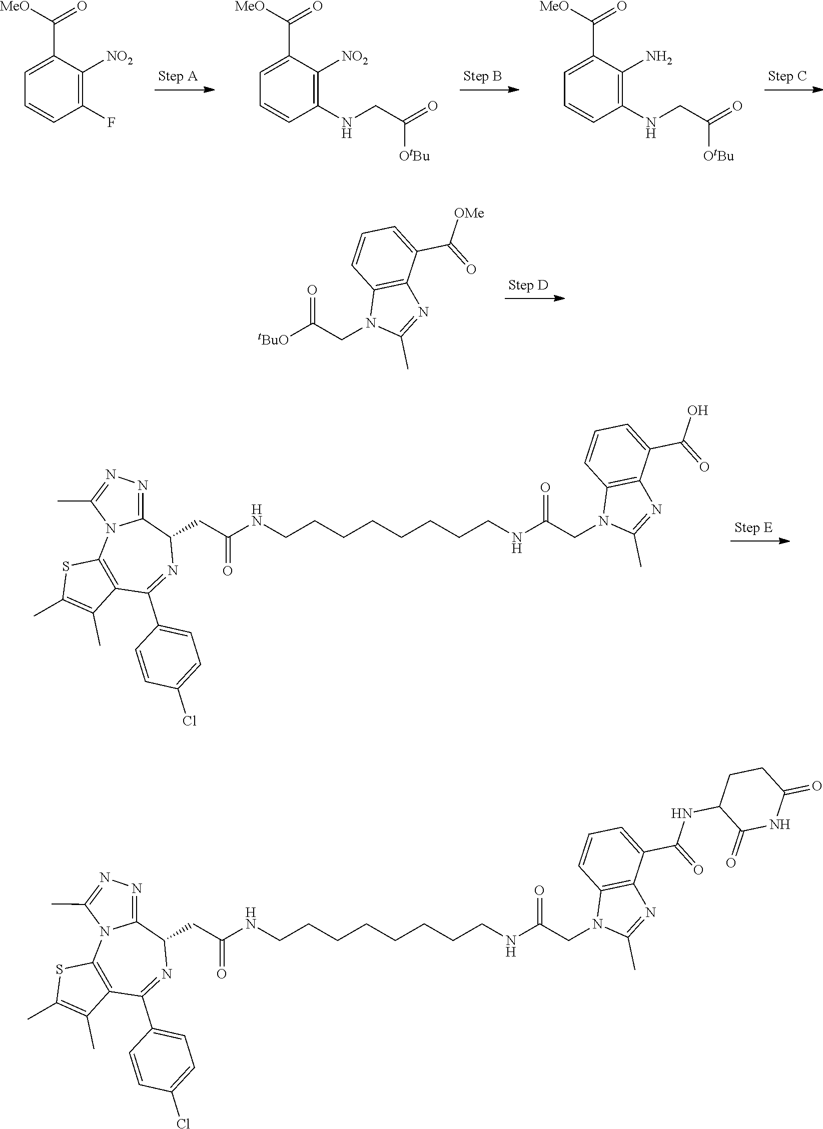 [SMILES: COC(=O)c1cccc(F)c1[N+](=O)[O-] ; COC(=O)c1cccc(NCC(=O)OC(C)(C)C)c1[N+](=O)[O-] ; COC(=O)c1cccc(NCC(=O)OC(C)(C)C)c1N ; COC(=O)c1cccc2c1nc(C)n2CC(=O)OC(C)(C)C ; Cc1sc2c(c1C)C(c1ccc(Cl)cc1)=N[C@@H](CC(=O)NCCCCCCCCNC(=O)Cn1c(C)nc3c(C(=O)O)cccc31)c1nnc(C)n1-2 ; Cc1sc2c(c1C)C(c1ccc(Cl)cc1)=N[C@@H](CC(=O)NCCCCCCCCNC(=O)Cn1c(C)nc3c(C(=O)NC4CCC(=O)NC4=O)cccc31)c1nnc(C)n1-2]

Step A: To a solution of methyl 3-fluoro-2-nitrobenzoate (150 mg, 0.753 mmol, 1 eq.) and glycine tert-butyl ester hydrochloride (429 mg, 2.56 mmol, 3.4 eq.) in acetonitrile (6 mL) was added DIPEA (0.656 mL, 3.75 mmol, 5 eq.) and the reaction mixture was stirred at 70° C. for 18 h. The solvent was removed under reduced pressure and the residue was purified by flash column chromatography to provide methyl 3-((2-(tert-butoxy)-2-oxoethyl)amino)-2-nitrobenzoate (149 mg, 63%).

Step B: Methyl 3-((2-(tert-butoxy)-2-oxoethyl)amino)-2-nitrobenzoate (70 mg, 0.226 mmol, 1 eq.) was dissolved in ethanol (5 mL) and water (2 mL). Iron powder (882 mg, 70 eq) was added followed by ammonium chloride (3.02 g, 250 eq) and the reaction mixture was stirred at 40° C. for 18 h. The reaction mixture was filtered, solids were washed with DCM and the filtrates were concentrated under reduced pressure. The crude product was purified by flash column chromatography to provide methyl 2-amino-3-((2-(tert-butoxy)-2-oxoethyl)amino)benzoate (36 mg, 56%).

Step C: Methyl 2-amino-3-((2-(tert-butoxy)-2-oxoethyl)amino)benzoate (110 mg, 0.393 mmol, 1 eq) was dissolved in hexafluoroisopropanol (4 mL). Ethyl orthoacetate (0.577 mL, 3.14 mmol, 8 eq) was added and the reaction mixture was stirred at RT for 60 h. The volatiles swerve removed under reduced pressure and the reaction mixture was purified by flash column chromatography to provide methyl 1-(2-(tert-butoxy)-2-oxoethyl)-2-methyl-1H-benzo[d]imidazole-4-carboxylate (89 mg, 74%).

Step D: Methyl 1-(2-(tert-butoxy)-2-oxoethyl)-2-methyl-1H-benzo[d]imidazole-4-carboxylate (30.4 mg, 0.100 mmol, 1 eq.) was dissolved in trifluoroacetic acid (3 mL) and the reaction mixture was stirred at RT for 18 h. The volatiles were removed under reduced pressure and dried under high vacuum. HATU (48.8 mg, 1.28 mmol, 1.28 eq), DMAP (1.3 mg, 0.011 mmol, 0.11 eq) and (S)—N-(8-aminooctyl)-2-(4-(4-chlorophenyl)-2,3,9-trimethyl-6H-thieno[3,2-f][1,2,4]triazolo[4,3-a][1,4]diazepin-6-yl)acetamide (50 mg, 0.11 mmol, 1.1 eq) were added, followed by DMF (12 mL) and DIPEA (0.225 mL, 1.28 mmol, 12 eq). The reaction mixture was stirred at RT for 6 h and the solvent was removed under reduced pressure. The solids were redissolved in methanol (4 mL) and water (1 mL) and lithium hydroxide (64 mg, 25 eq) was added. The mixture was stirred for 72 h at RT. 1M HCl was added to acidify the mixture, the solvent was evaporated and the residue was purified by HPLC to provide (S)-1-(2-((8-(2-(4-(4-chlorophenyl)-2,3,9-trimethyl-6H-thieno[3,2-f][1,2,4]triazolo[4,3-a][1,4]diazepin-6-yl)acetamido)octyl)amino)-2-oxoethyl)-2-methyl-1H-benzo[d]imidazole-4-carboxylic acid (69.4 mg, 87%).

Step E: (S)-1-(2-((8-(2-(4-(4-chlorophenyl)-2,3,9-trimethyl-6H-thieno[3,2-f][1,2,4]triazolo[4,3-a][1,4]diazepin-6-yl)acetamido)octyl)amino)-2-oxoethyl)-2-methyl-1H-benzo[d]imidazole-4-carboxylic acid (14 mg, 0.019 mmol, 1 eq), 3-aminopiperidine-2,6-dione hydrochloride (18.6 mg, 0.113 mmol, 1.2 eq), HATU (43 mg, 0.113 mmol, 1.2 eq) and DMAP (0.5 mg, 0.004 mmol, 0.1 eq) were dissolved in NMP (2 mL). DIPEA (0.098 mL, 0.565 mmol, 30 eq) was added and the reaction mixture was stirred at RT for 3 h. The reaction mixture was purified by HPLC to provide 1-(2-((8-(2-((S)-4-(4-chlorophenyl)-2,3,9-trimethyl-6H-thieno[3,2-f][1,2,4]triazolo[4,3-a][1,4]diazepin-6-yl)acetamido)octyl)amino)-2-oxoethyl)-N-(2,6-dioxopiperidin-3-yl)-2-methyl-1H-benzo[d]imidazole-4-carboxamide (6.4 mg, 39%).

¹H NMR (500 MHz, DMSO) δ 10.91 (s, 1H), 10.23 (d, J=7.3 Hz, 1H), 8.33 (t, J=5.6 Hz, 1H), 8.15 (q, J=5.4 Hz, 1H), 7.85 (dd, J=7.6, 1.0 Hz, 1H), 7.64 (dd, J=8.1, 1.0 Hz, 1H), 7.48 (dd, J=8.8, 3.1 Hz, 3H), 7.42 (dd, J=8.7, 2.0 Hz, 3H), 7.31 (t, J=7.8 Hz, 1H), 4.94 (s, 2H), 4.89 (ddd, J=12.6, 9.0, 5.3 Hz, 1H), 4.50 (dd, J=8.1, 6.1 Hz, 1H), 2.86-2.77 (m, 2H), 2.58 (d, J=5.1 Hz, 3H), 2.56 (d, J=4.0 Hz, 3H), 2.40 (d, J=0.5 Hz, 3H), 2.28-2.21 (m, 1H), 2.18-2.07 (m, 1H), 1.61 (s, 3H), 1.47-1.36 (m, 5H), 1.33-1.19 (m, 12H).

LCMS (m/z [M+H]⁺): 853.9

### Example 58: Synthesis of 5-(2-((8-(2-((S)-4-(4-chlorophenyl)-2,3,9-trimethyl-6H-thieno[3,2-f][1,2,4]triazolo[4,3-a][1,4]diazepin-6-yl)acetamido)octyl)amino)-2-oxoethoxy)-N-(2,6-dioxopiperidin-3-yl)-2-methyl-1H-benzo[d]imidazole-7-carboxamide (66)

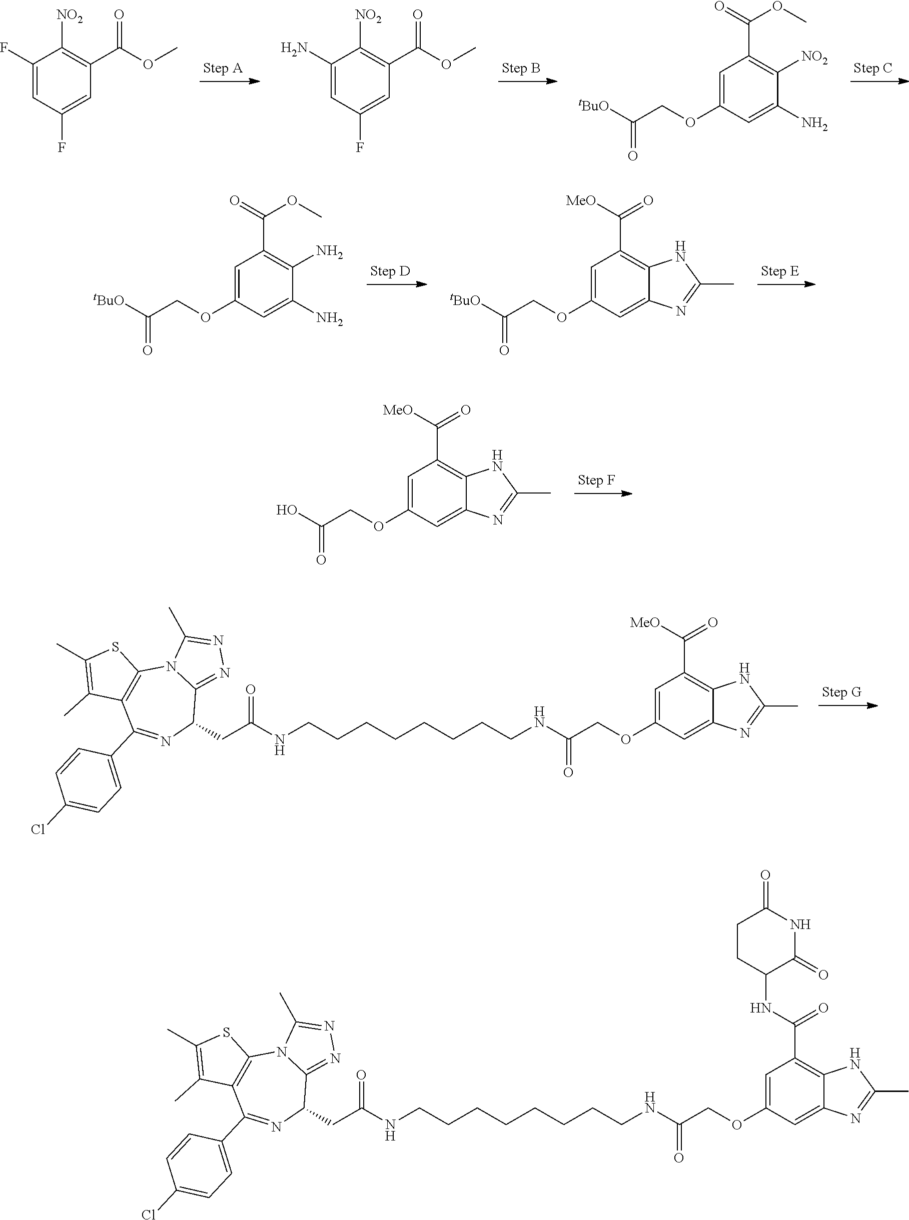

Step A: Methyl 3,5-difluoro-2-nitro-benzoate (10 g, 46.083 mmol) was dissolved in DMF and treated with ammonium carbonate (5.3 g, 55.3 mmol). The reaction was heated at 60° C. for 6 h. The reaction mixture was diluted with ethyl acetate and washed successively with water and brine. The organic layer was dried over Na₂SO₄ and concentrated under reduced pressure to get the crude product, which was purified by flash column chromatography to give methyl 3-amino-5-fluoro-2-nitro-benzoate 7.6 g (77%).

Step B: Sodium hydride (706 mg, 17.674 mmol) was added to a DMF (100 ml) solution of tert-butyl 2-hydroxyacetate (2.4 g, 18.6 mmol) at 0° C. under nitrogen. The reaction mixture was allowed to stir at 0° C. for 30 min. To the mixture was added methyl 3-amino-5-fluoro-2-nitro-benzoate (2 g, 9.302 mmol) at 0° C. The resulting mixture was stirred at RT for 1.5 h. The reaction mixture was then cooled down to 0° C., quenched by adding saturated ammonium chloride solution, diluted with ethyl acetate and washed with water. The organic layer was dried over Na₂SO₄ and concentrated under reduced pressure to get crude product, which was purified by flash column chromatography to give methyl 3-amino-5-(2-(tert-butoxy)-2-oxoethoxy)-2-nitrobenzoate 1.5 g (49%).

Step C: Methyl 3-amino-5-(2-(tert-butoxy)-2-oxoethoxy)-2-nitrobenzoate (1.5 g, 4.6 mmol) was dissolved in methanol (30 mL), the reaction mixture was deoxygenated using argon balloon and palladium on charcoal (75 mg) was added. The reaction vessel was backfilled with hydrogen (1 bar) and stirred at RT for 18 h and filtered over the celite. The filtrate was concentrated under reduced pressure and the residue was purified was purified by flash column chromatography to give methyl 2,3-diamino-5-(2-(tert-butoxy)-2-oxoethoxy)benzoate 900 mg (66%).

Step D: To an aqueous solution of sodium bisulfite (40% in water, 15 mL, and 4.561 mmol) was added methyl 2,3-diamino-5-(2-(tert-butoxy)-2-oxoethoxy)benzoate (900 mg, 3.041 mmol) followed by a solution of acetaldehyde (0.3 ml, 4.561 mmol) in ethanol (15 mL). The reaction mixture was heated to reflux for 4 h. Volatiles were removed under reduced pressure, diluted with dichloromethane and washed with water and brine. The organic layer extract was dried over Na₂SO₄ and concentrated under reduced pressure to get crude product, which was purified by flash column chromatography to give methyl 6-(2-(tert-butoxy)-2-oxoethoxy)-2-methyl-1H-benzo[d]imidazole-4-carboxylate 400 mg (40%).

Step E: Methyl 6-(2-(tert-butoxy)-2-oxoethoxy)-2-methyl-1H-benzo[d]imidazole-4-carboxylate (400 mg, 1.25 mmol) was suspended in dioxane (5 mL) and cooled to 0° C. 4M HCl in dioxane (4 mL) was added dropwise and the reaction mixture was allowed to stir at room temperature for 16 h. The volatiles were removed under reduced pressure and the product was triturated with ether and pentane to give 2-((4-(methoxycarbonyl)-2-methyl-1H-benzo[d]imidazol-6-yl)oxy)acetic acid 300 mg (91%).

Step F: To a solution of (S)—N-(8-aminooctyl)-2-(4-(4-chlorophenyl)-2,3,9-trimethyl-6H-thieno[3,2-f][1,2,4]triazolo[4,3-a][1,4]diazepin-6-yl)acetamide (55 mg, 0.098 mmol, 1 eq), 2-((7-(methoxycarbonyl)-2-methyl-1H-benzo[d]imidazol-5-yl)oxy)acetic acid (31 mg, 0.117 mmol, 1.2 eq), HATU (260 mg, 0.976 mmol, 7 eq) in DMF (3 mL) was added DIPEA (0.170 mL, 0.976 mmol, 10 eq) and the reaction mixture was stirred at RT for 20 h. The solvent was removed under reduced pressure and the residue was purified by flash column chromatography to give methyl (S)-5-(2-((8-(2-(4-(4-chlorophenyl)-2,3,9-trimethyl-6H-thieno[3,2-f][1,2,4]triazolo[4,3-α][1,4]diazepin-6-yl)acetamido)octyl)amino)-2-oxoethoxy)-2-methyl-1H-benzo[d]imidazole-7-carboxylate (35 mg, 46%).

Step G: To a solution of methyl (S)-5-(2-((8-(2-(4-(4-chlorophenyl)-2,3,9-trimethyl-6H-thieno[3,2-f][1,2,4]triazolo[4,3-α][1,4]diazepin-6-yl)acetamido)octyl)amino)-2-oxoethoxy)-2-methyl-1H-benzo[d]imidazole-7-carboxylate (34 mg, 0.044 mmol, 1 eq) in methanol (2 mL) was added sodium hydroxide (2.3 ml, 1M) and the reaction mixture was stirred at RT for 20 h. 1M HCl was added to neutralize the base and the mixture was evaporated under reduced pressure. To the residue was added 3-aminopiperidine-2,6-dione hydrochloride (37 mg, 0.224 mmol, 5 eq), HATU (34 mg, 0.090 mmol, 2 eq) and NMP (1 mL). DIPEA (0.023 mL, 0.134 mmol, 3 eq) was added and the reaction mixture was stirred at RT for 20 h. The reaction mixture was purified by HPLC to provide 5-(2-((8-(2-((S)-4-(4-chlorophenyl)-2,3,9-trimethyl-6H-thieno[3,2-f][1,2,4]triazolo[4,3-α][1,4]diazepin-6-yl)acetamido)octyl)amino)-2-oxoethoxy)-N-(2,6-dioxopiperidin-3-yl)-2-methyl-1H-benzo[d]imidazole-7-carboxamide 26 mg (65%).

¹H NMR (500 MHz, DMSO) δ 12.57 (s, 1H), 10.90 (s, 1H), 10.25 (d, J=7.3 Hz, 1H), 8.12 (dd, J=13.5, 5.8 Hz, 2H), 7.52-7.40 (m, 5H), 7.18 (d, J=2.5 Hz, 1H), 4.87 (ddd, J=12.6, 7.2, 5.4 Hz, 1H), 4.53-4.46 (m, 3H), 3.21 (ddd, J=21.0, 15.0, 7.1 Hz, 3H), 3.08 (ddd, J=18.9, 13.1, 6.3 Hz, 4H), 2.82 (ddd, J=18.5, 15.9, 8.7 Hz, 1H), 2.59 (s, 3H), 2.53 (s, 3H), 2.40 (d, J=0.5 Hz, 3H), 2.27-2.17 (m, 1H), 2.11 (qd, J=12.9, 3.8 Hz, 1H), 1.61 (s, 3H), 1.41 (d, J=6.5 Hz, 4H), 1.22 (d, J=14.5 Hz, 8H).

LCMS (m/z [M+H]⁺): 869.9

### Example 59: Synthesis of 6-(2-((8-(2-((S)-4-(4-chlorophenyl)-2,3,9-trimethyl-6H-thieno[3,2-f][1,2,4]triazolo[4,3-a][1,4]diazepin-6-yl)acetamido)octyl)amino)-2-oxoethoxy)-N-(2,6-dioxopiperidin-3-yl)-2-methyl-1H-benzo[d]imidazole-7-carboxamide (67)

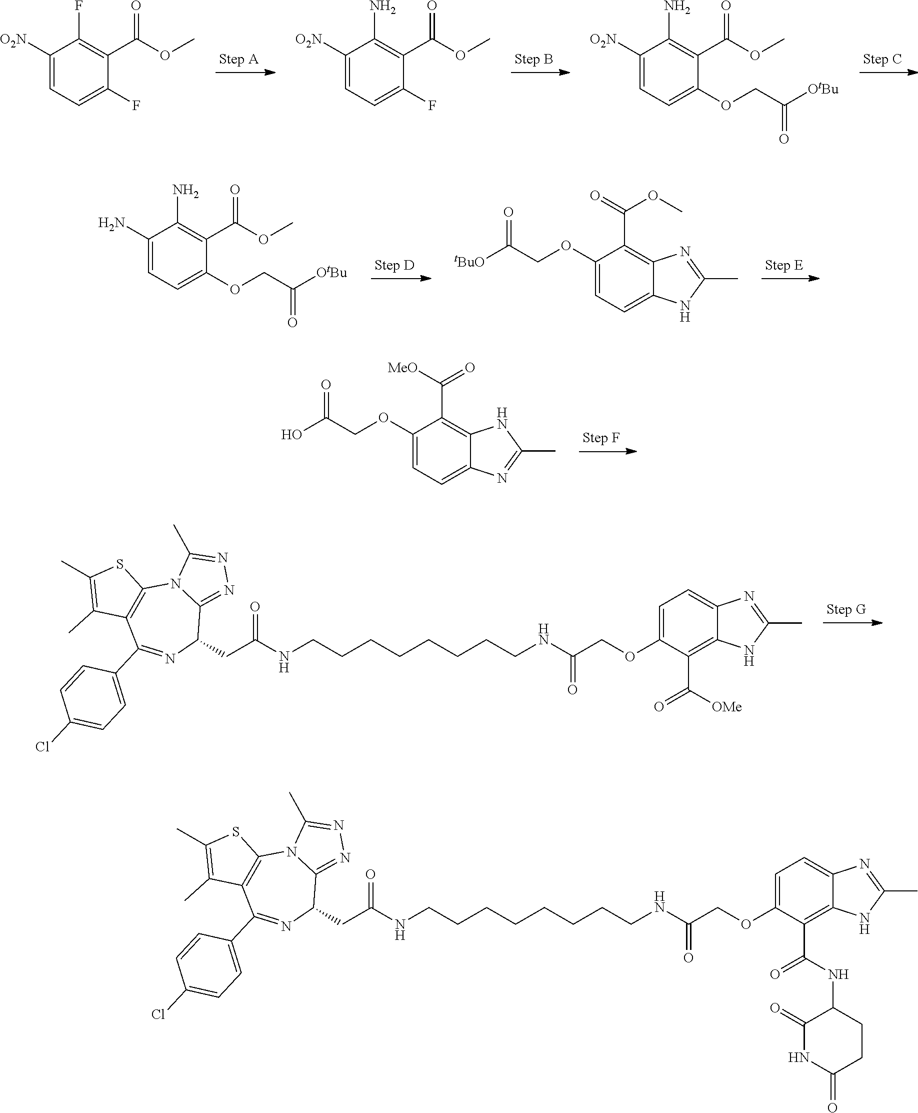

Step A: Methyl 2,6-difluoro-3-nitro-benzoate (10 g, 46.08 mmol) was dissolved in DMF and treated with ammonium carbonate (5.3 g, 55.3 mmol). The reaction was heated at 60° C. for 6 h. The reaction mixture was diluted with ethyl acetate and washed successively with water and brine. The organic layer was dried over Na₂SO₄ and concentrated under reduced pressure to get the crude product, which was purified by flash column chromatography to give methyl 2-amino-6-fluoro-3-nitro-benzoate 5.1 g (51%).

Step B: Sodium hydride (896 mg, 22.43 mmol) was added to a DMF (100 ml) solution of tert-butyl 2-hydroxyacetate (3.1 g, 23.3 mmol) at 0° C. under nitrogen. The reaction mixture was allowed to stir at 0° C. for 30 min and methyl 2-amino-6-fluoro-3-nitro-benzoate (2 g, 9.302 mmol) was added at 0° C. The resulting mixture was stirred at RT for 1.5 h. The reaction mixture was then cooled down to 0° C., quenched by adding saturated ammonium chloride solution, diluted with ethyl acetate and washed with water. The organic layer was dried over Na₂SO₄ and concentrated under reduced pressure to get crude product, which was purified by flash column chromatography to give methyl 2-amino-6-(2-(tert-butoxy)-2-oxoethoxy)-3-nitrobenzoate 700 mg (23%).

Step C: Methyl 2-amino-6-(2-(tert-butoxy)-2-oxoethoxy)-3-nitrobenzoate (700 mg, 2.14 mmol) was dissolved in methanol (30 mL). The reaction mixture was deoxygenated using argon balloon and palladium on charcoal (70 mg) was added. The reaction vessel was backfilled with hydrogen (1 bar) and stirred at RT for 18 h and filtered over the celite. The filtrate was concentrated under reduced pressure and the residue was purified by flash column chromatography to give methyl 2,3-diamino-6-(2-(tert-butoxy)-2-oxoethoxy)-benzoate 600 mg (94%).

Step D: To an aqueous solution of sodium bisulfite (40% in water, 15 mL, and 3.041 mmol) was added methyl 2,3-diamino-6-(2-(tert-butoxy)-2-oxoethoxy)-benzoate (600 mg, 2.027 mmol) followed by a solution of acetaldehyde (0.2 ml, 3.041 mmol) in ethanol (15 mL). The reaction mixture was heated to reflux for 4 h. Volatiles were removed under reduced pressure, diluted with dichloromethane and washed with water and brine. The organic layer extract was dried over Na₂SO₄ and concentrated under reduced pressure to get crude product, which was purified by flash column chromatography to give methyl 5-(2-(tert-butoxy)-2-oxoethoxy)-2-methyl-1H-benzo[d]imidazole-4-carboxylate 400 mg (61%).

Step E: Methyl 5-(2-(tert-butoxy)-2-oxoethoxy)-2-methyl-1H-benzo[d]imidazole-4-carboxylate (400 mg, 1.25 mmol, 1 equiv) was suspended in dioxane (5 mL) and cooled to 0° C. 4M HCl in dioxane (4 mL) was added dropwise and the reaction mixture was allowed to stir at room temperature for 16 h. The volatiles were removed under reduced pressure and the product was triturated with ether and pentane to give 2-((7-(methoxycarbonyl)-2-methyl-1H-benzo[d]imidazol-6-yl)oxy)acetic acid 280 mg (84%).

Step F: To a solution of (S)—N-(8-aminooctyl)-2-(4-(4-chlorophenyl)-2,3,9-trimethyl-6H-thieno[3,2-f][1,2,4]triazolo[4,3-α][1,4]diazepin-6-yl)acetamide (55 mg, 0.098 mmol, 1 eq), 2-((7-(methoxycarbonyl)-2-methyl-1H-benzo[d]imidazol-6-yl)oxy)acetic acid (31 mg, 0.117 mmol, 1.2 eq), HATU (260 mg, 0.976 mmol, 7 eq) in DMF (3 mL) was added DIPEA (0.170 mL, 0.976 mmol, 10 eq) and the reaction mixture was stirred at RT for 20 h. The solvent was removed under reduced pressure and the residue was purified by flash column chromatography to give methyl (S)-6-(2-((8-(2-(4-(4-chlorophenyl)-2,3,9-trimethyl-6H-thieno[3,2-f][1,2,4]triazolo[4,3-α][1,4]diazepin-6-yl)acetamido)octyl)amino)-2-oxoethoxy)-2-methyl-1H-benzo[d]imidazole-7-carboxylate 36 mg (47%).

Step G: To a solution of methyl (S)-6-(2-((8-(2-(4-(4-chlorophenyl)-2,3,9-trimethyl-6H-thieno[3,2-f][1,2,4]triazolo[4,3-α][1,4]diazepin-6-yl)acetamido)octyl)amino)-2-oxoethoxy)-2-methyl-1H-benzo[d]imidazole-7-carboxylate (35 mg, 0.045 mmol, 1 eq) in methanol (2 mL) was added sodium hydroxide (2.3 ml, 1M) and the reaction mixture was stirred at RT for 20 h. 1M HCl was added to neutralize the base and the mixture was evaporated under reduced pressure. To the residue was added 3-aminopiperidine-2,6-dione hydrochloride (37 mg, 0.224 mmol, 5 eq), HATU (34 mg, 0.090 mmol, 2 eq) and NMP (1 mL). DIPEA (0.023 mL, 0.134 mmol, 3 eq) was added and the reaction mixture was stirred at RT for 20 h. The reaction mixture was purified by HPLC to provide 6-(2-((8-(2-((S)-4-(4-chlorophenyl)-2,3,9-trimethyl-6H-thieno[3,2-f][1,2,4]triazolo[4,3-α][1,4]diazepin-6-yl)acetamido)octyl)amino)-2-oxoethoxy)-N-(2,6-dioxopiperidin-3-yl)-2-methyl-1H-benzo[d]imidazole-7-carboxamide 24 mg (60%).

¹H NMR (500 MHz, DMSO) δ 12.06 (s, 1H), 10.87 (s, 1H), 9.45 (d, J=7.9 Hz, 1H), 8.24-8.06 (m, 2H), 7.60 (d, J=8.7 Hz, 1H), 7.48 (d, J=8.8 Hz, 2H), 7.42 (d, J=8.6 Hz, 2H), 6.86 (d, J=8.8 Hz, 1H), 4.84-4.75 (m, 1H), 4.73-4.54 (m, 2H), 4.50 (dd, J=8.1, 6.1 Hz, 1H), 3.21 (ddd, J=21.0, 15.0, 7.1 Hz, 3H), 3.15-3.03 (m, 4H), 2.87-2.77 (m, 1H), 2.59 (s, 3H), 2.48 (s, 3H), 2.40 (d, J=0.5 Hz, 3H), 2.27 (qd, J=13.0, 4.4 Hz, 1H), 2.12-2.05 (m, 1H), 1.62 (d, J=0.5 Hz, 3H), 1.48-1.35 (m, 4H), 1.23 (s, 8H).

LCMS (m/z [M+H]⁺): 868.8

### Example 60: Fluorescence Polarization (FP) Assays

CRBN-DDB1 protein complex was mixed with Cy5-labelled thalidomide and a compound to be tested (the “test compound”). The test solution contained 50 mM Tris pH=7.0, 200 mM NaCl, 0.02% v/v Tween-20, 2 mM DTT, 5 nM Cy5-labelled thalidomide (the tracer), 25 nM CRBN-DDB1 protein, 2% v/v DMSO. The test solution was added to a 384-well assay plate.

The plate was spun-down (1 min, 1000 rpm, 22° C.) and then shaken using a VibroTurbulator for 10 min at room temperature (20-25° C.), with the frequency set to level 3. The assay plate with protein and the tracer was incubated for 60 min at room temperature (20-25° C.) prior to read-out with a plate reader. Read-out (fluorescence polarization) was performed by a Pherastar plate reader, using a Cy5 FP Filterset (590 nm/675 nm).

The FP experiment was carried out with various concentrations of the test compounds in order to measure Kᵢ values.

The Kᵢ values of competitive inhibitors were calculated using the equation based on the IC₅₀ values of relationship between compound concentration and measured fluorescence polarization, the K_{d} value of the Cy5-T and CRBN/DDB1 complex, and the concentrations of the protein and the tracer in the displacement assay (as described by Z. Nikolovska-Coleska et al., Analytical Biochemistry 332 (2004) 261-273).

### Fluorescence Polarization (FP) Assay—Results

Compounds are categorized based on their affinity to CRBN defined as Ki. As reported in Table 1, below, the compounds of the present invention interact with CRBN-DDB1 protein within similar affinity range as reported for reference compounds.

TABLE 1
FP assay results for compounds of the present invention and control compounds CC-122,
lenalidomide and pomalidomide.

| Compound ID | Structure | CRBN binding Ki [μM]* |
|---|---|---|
| CC-122 | 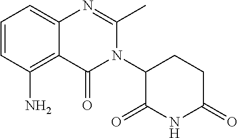 [SMILES: Cc1nc2cccc(N)c2c(=O)n1C1CCC(=O)NC1=O] | B |
| Pomalidomide | 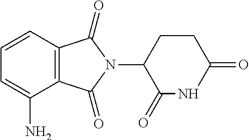 [SMILES: Nc1cccc2c1C(=O)N(C1CCC(=O)NC1=O)C2=O] | A |
| Lenalidomide | 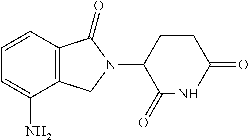 [SMILES: Nc1cccc2c1CN(C1CCC(=O)NC1=O)C2=O] | A |
| 1 | 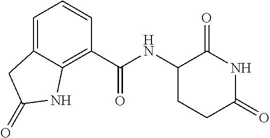 [SMILES: O=C1CCC(NC(=O)c2cccc3c2NC(=O)C3)C(=O)N1] | C |
| 2 | 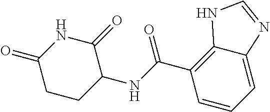 [SMILES: O=C1CCC(NC(=O)c2cccc3nc[nH]c23)C(=O)N1] | B |
| 4 | 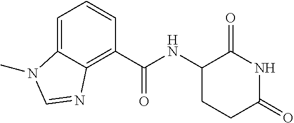 [SMILES: Cn1cnc2c(C(=O)NC3CCC(=O)NC3=O)cccc21] | B |
| 7 | 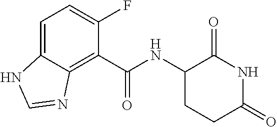 [SMILES: O=C1CCC(NC(=O)c2c(F)ccc3[nH]cnc23)C(=O)N1] | B |
| 8 | 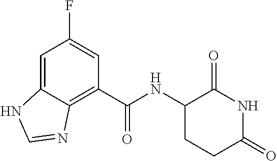 [SMILES: O=C1CCC(NC(=O)c2cc(F)cc3[nH]cnc23)C(=O)N1] | B |
| 9 | 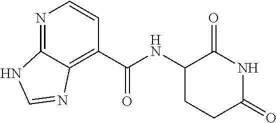 [SMILES: O=C1CCC(NC(=O)c2ccnc3[nH]cnc23)C(=O)N1] | B |
| 10 | 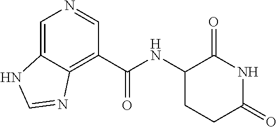 [SMILES: O=C1CCC(NC(=O)c2cncc3[nH]cnc23)C(=O)N1] | B |
| 11 | 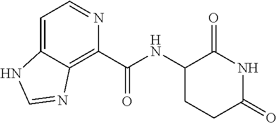 [SMILES: O=C1CCC(NC(=O)c2nccc3[nH]cnc23)C(=O)N1] | B |
| 12 | 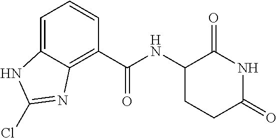 [SMILES: O=C1CCC(NC(=O)c2cccc3[nH]c(Cl)nc23)C(=O)N1] | A |
| 15 | 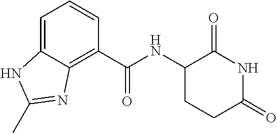 [SMILES: Cc1nc2c(C(=O)NC3CCC(=O)NC3=O)cccc2[nH]1] | A |
| 22 | 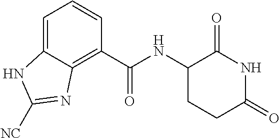 [SMILES: N#Cc1nc2c(C(=O)NC3CCC(=O)NC3=O)cccc2[nH]1] | A |
| 23 | 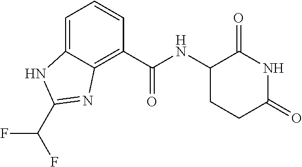 [SMILES: O=C1CCC(NC(=O)c2cccc3[nH]c(C(F)F)nc23)C(=O)N1] | B |
| 24 | 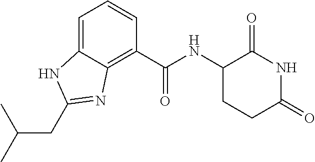 [SMILES: CC(C)Cc1nc2c(C(=O)NC3CCC(=O)NC3=O)cccc2[nH]1] | B |
| 25 | 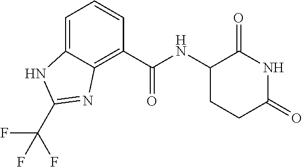 [SMILES: O=C1CCC(NC(=O)c2cccc3[nH]c(C(F)(F)F)nc23)C(=O)N1] | A |
| 26 | 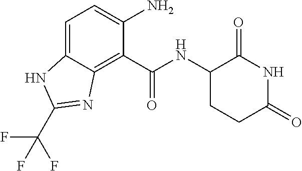 [SMILES: Nc1ccc2[nH]c(C(F)(F)F)nc2c1C(=O)NC1CCC(=O)NC1=O] | B |
| 30 | 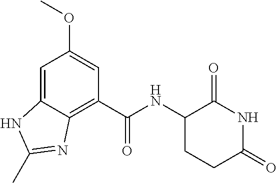 [SMILES: COc1cc(C(=O)NC2CCC(=O)NC2=O)c2nc(C)[nH]c2c1] | A |
| 35 | 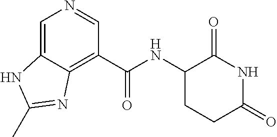 [SMILES: Cc1nc2c(C(=O)NC3CCC(=O)NC3=O)cncc2[nH]1] | B |
| 36 | 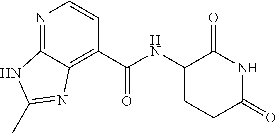 [SMILES: Cc1nc2c(C(=O)NC3CCC(=O)NC3=O)ccnc2[nH]1] | B |
| 37 | 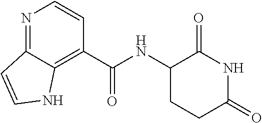 [SMILES: O=C1CCC(NC(=O)c2ccnc3cc[nH]c23)C(=O)N1] | C |
| 39 | 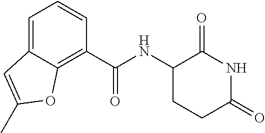 [SMILES: Cc1cc2cccc(C(=O)NC3CCC(=O)NC3=O)c2o1] | A |
| 41 | 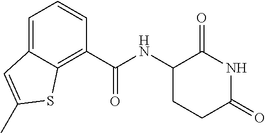 [SMILES: Cc1cc2cccc(C(=O)NC3CCC(=O)NC3=O)c2s1] | B |
| 42 | 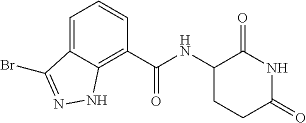 [SMILES: O=C1CCC(NC(=O)c2cccc3c(Br)n[nH]c23)C(=O)N1] | C |
| 43 | 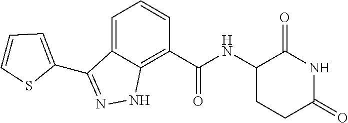 [SMILES: O=C1CCC(NC(=O)c2cccc3c(-c4cccs4)n[nH]c23)C(=O)N1] | B |
| 47 | 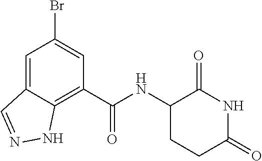 [SMILES: O=C1CCC(NC(=O)c2cc(Br)cc3cn[nH]c23)C(=O)N1] | B |
| 48 | 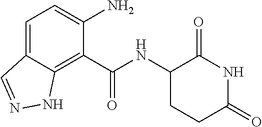 [SMILES: Nc1ccc2cn[nH]c2c1C(=O)NC1CCC(=O)NC1=O] | B |
| 50 | 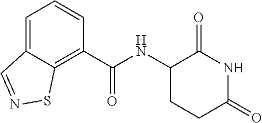 [SMILES: O=C1CCC(NC(=O)c2cccc3cnsc23)C(=O)N1] | B |
| 51 | 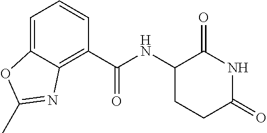 [SMILES: Cc1nc2c(C(=O)NC3CCC(=O)NC3=O)cccc2o1] | B |
| 52 | 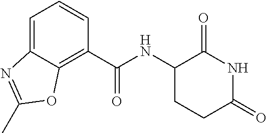 [SMILES: Cc1nc2cccc(C(=O)NC3CCC(=O)NC3=O)c2o1] | B |
| 53 | 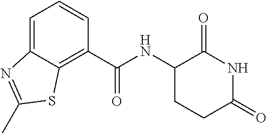 [SMILES: Cc1nc2cccc(C(=O)NC3CCC(=O)NC3=O)c2s1] | B |
| 54 | 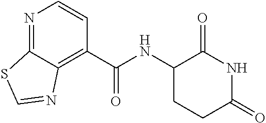 [SMILES: O=C1CCC(NC(=O)c2ccnc3scnc23)C(=O)N1] | B |
| 55 | 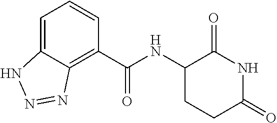 [SMILES: O=C1CCC(NC(=O)c2cccc3[nH]nnc23)C(=O)N1] | B |
| 58 | 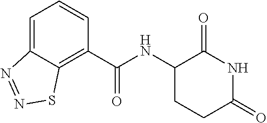 [SMILES: O=C1CCC(NC(=O)c2cccc3nnsc23)C(=O)N1] | C |
| 60 | 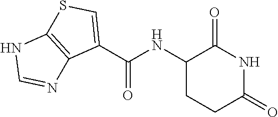 [SMILES: O=C1CCC(NC(=O)c2csc3[nH]cnc23)C(=O)N1] | B |
| 62 | 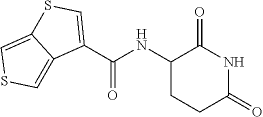 [SMILES: O=C1CCC(NC(=O)c2csc3cscc23)C(=O)N1] | B |
| 63 | 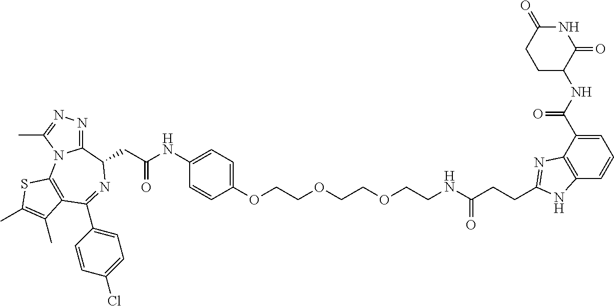 [SMILES: Cc1sc2c(c1C)C(c1ccc(Cl)cc1)=N[C@@H](CC(=O)Nc1ccc(OCCOCCOCCNC(=O)CCc3nc4c(C(=O)NC5CCC(=O)NC5=O)cccc4[nH]3)cc1)c1nnc(C)n1-2] | B |
| 64 | 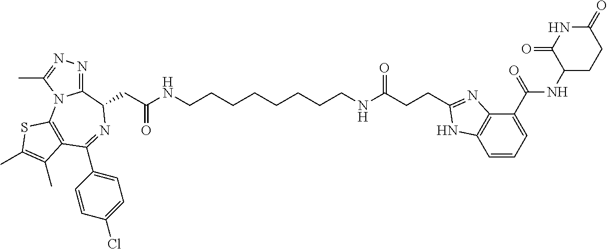 [SMILES: Cc1sc2c(c1C)C(c1ccc(Cl)cc1)=N[C@@H](CC(=O)NCCCCCCCCNC(=O)CCc1nc3c(C(=O)NC4CCC(=O)NC4=O)cccc3[nH]1)c1nnc(C)n1-2] | B |
| 65 | 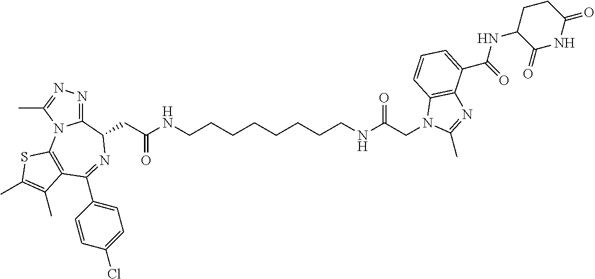 [SMILES: Cc1sc2c(c1C)C(c1ccc(Cl)cc1)=N[C@@H](CC(=O)NCCCCCCCCNC(=O)Cn1c(C)nc3c(C(=O)NC4CCC(=O)NC4=O)cccc31)c1nnc(C)n1-2] | A |
| 66 | 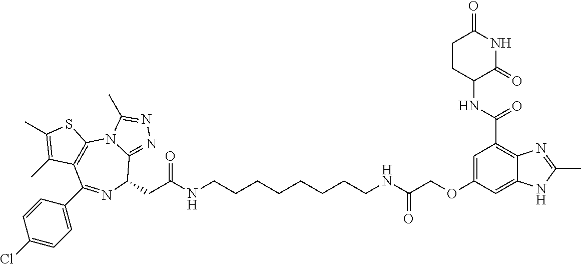 [SMILES: Cc1nc2c(C(=O)NC3CCC(=O)NC3=O)cc(OCC(=O)NCCCCCCCCNC(=O)C[C@@H]3N=C(c4ccc(Cl)cc4)c4c(sc(C)c4C)-n4c(C)nnc43)cc2[nH]1] | A |
| 67 | 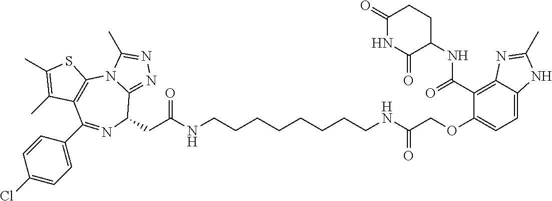 [SMILES: Cc1nc2c(C(=O)NC3CCC(=O)NC3=O)c(OCC(=O)NCCCCCCCCNC(=O)C[C@@H]3N=C(c4ccc(Cl)cc4)c4c(sc(C)c4C)-n4c(C)nnc43)ccc2[nH]1] | B |

*CRBN binding Ki [μM] A ≤ 1; 1 < B ≤ 10, 10 < C ≤ 50

### Example 61: SALL4 Degradation Assay—Kelly Cell Line

The effect of various compounds of the invention and various reference compounds on SALL4 degradation in the Kelly cell line was investigated, using the degradation assay protocol below.

Kelly cells were maintained in RPMI-1640 medium, supplemented with penicillin/streptomycin and 10% Fetal Bovine Serum (FBS). Cells were seeded on 6-well plates, and the compounds to be tested were added at the desired concentration range. Final DMSO concentration was 0.25%. After 24 h incubation (37° C., 5% CO₂), cells were washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were: LENALIDOMIDE, POMALIDOMIDE, 39, 35 and 50 at the concentrations 10 and 20 μM, and a group of compounds listed in the Table 3 at the concentration of 20 μM; the treatment with all compounds was carried out for 24 h. Densitometry values are normalized to the loading control (β-ACTIN) and presented as % of DMSO control, using the following labels:
≤25% for 0-25% of SALL4 protein reduction,
>25% for 26-74% of SALL4 protein reduction,
≥75% for 75-100% of SALL4 protein reduction.

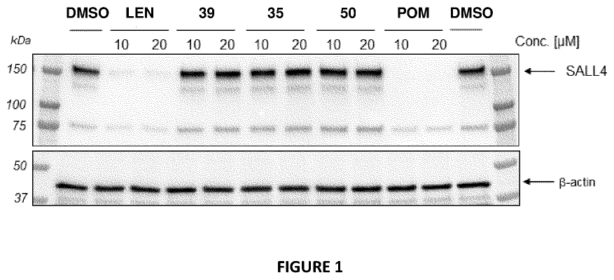

The representative results for compounds: LENALIDOMIDE, 39, 35, 50 and POMALIDOMIDE are shown in FIG. 1 and Table 2. The remaining compounds are presented in Table 10. As illustrated in FIG. 1 and Table 2 and 10, the compounds of the present invention do not possess the capability of SALL4 degradation, unlike the reference compounds LENALIDOMIDE or POMALIDOMIDE.

TABLE 2
SALL4 degradation in Kelly cell line. Cells were treated with the compounds:
LENALIDOMIDE, 39, 35, 50 and POMALIDOMIDE at the concentrations 10 and 20 μM for 24 h.
% of SALL4 protein reduction is provided based on normalized densitometry values.
% of SALL4 protein reduction, based on densitometry values

| | DMSO | LENALIDOMIDE | | 39 | | 35 | | 50 | | POMALIDOMIDE | |
|---|---|---|---|---|---|---|---|---|---|---|---|
| [μM] | 0.25% | 10 | 20 | 10 | 20 | 10 | 20 | 10 | 20 | 10 | 20 |
| SALL4 | 0% | ≥75% | ≥75% | ≤25% | ≤25% | ≤25% | ≤25% | ≤25% | ≤25% | ≥75% | ≥75% |

TABLE 3
The list of compounds used in the SALL4 and CK1α degradation
assay at the concentration of 20 μM.

1
3
4
5
6
7
8
15
20
23
24
25
30
36
37

### Example 62: CK1a Degradation Assay—Kelly Cell Line

The effect of various compounds of the invention and various reference compounds on CK1a degradation in the Kelly cell line was investigated, using the degradation assay protocol below.

Kelly cells were maintained in RPMI-1640 medium, supplemented with penicillin/streptomycin and 10% Fetal Bovine Serum (FBS). Cells were seeded on 6-well plates, and the compounds to be tested were added at the desired concentration range. Final DMSO concentration was 0.25%. After 24 h incubation (37° C., 5% CO₂), cells were washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were: LENALIDOMIDE, POMALIDOMIDE, 39, 35 and 50 at the concentrations 10 and 20 μM, and a group of compounds listed in the Table 3 at the concentration of 20 μM; the treatment with all compounds was carried out for 24 h. Densitometry values are normalized to the loading control (β-ACTIN) and presented as % of DMSO control, using the following labels:
≤25% for 0-25% of CK1a protein reduction,
>25% for 26-74% of CK1a protein reduction,
≥75% for 75-100% of CK1a protein reduction.

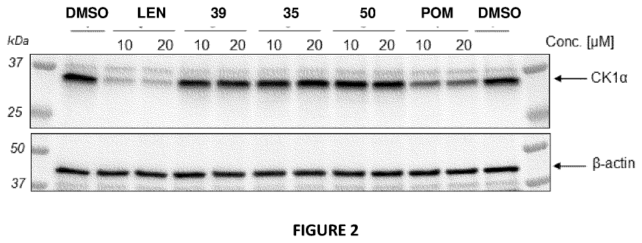

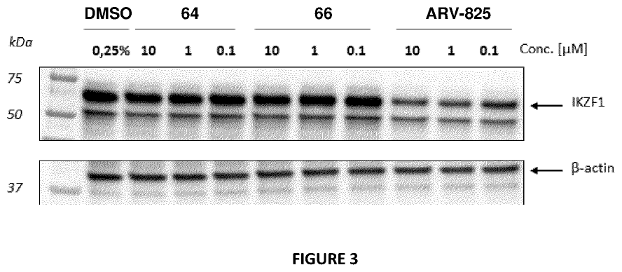

The representative results for compounds: LENALIDOMIDE, 39, 35, 50 and POMALIDOMIDE are shown in FIG. 2 and Table 4. The remaining compounds are presented in Table 10. As illustrated in FIG. 2 and Table 4 and 10, the compounds of the present invention do not induce the CK1a degradation in Kelly cell line, which is degraded by the reference compounds: LENALIDOMIDE or, to a lesser degree, by POMALIDOMIDE.

TABLE 4
CK1α degradation in Kelly cell line. Cells were treated with the compounds:
LENALIDOMIDE, 39, 35, 50 and POMALIDOMIDE at the concentrations 10 and 20 μM for 24 h.
% of CK1α protein reduction is provided based on normalized densitometry values.
% of CK1α protein reduction, based on densitometry values

| | DMSO | LENALIDOMIDE | | 39 | | 35 | | 50 | | POMALIDOMIDE | |
|---|---|---|---|---|---|---|---|---|---|---|---|
| [μM] | 0.25% | 10 | 20 | 10 | 20 | 10 | 20 | 10 | 20 | 10 | 20 |
| CK1α | 0% | >25% | >25% | ≤25% | ≤25% | ≤25% | ≤25% | ≤25% | ≤25% | >25% | >25% |

### Example 63: IKZF1 Degradation Assay—H929 Cell Line

The effect of various compounds of the invention and various reference compounds on IKZF1 degradation in the H929 cell line was investigated, using the degradation assay protocol below.

H929 cells were maintained in RPM1-1640 medium, supplemented with penicillin/streptomycin, 10% Fetal Bovine Serum (FBS) and 0.05 mM 2-Mercaptoethanol. Cells were seeded on 6- or 12-well plates, and the compounds to be tested were added at the desired concentration range. Final DMSO concentration was 0.25%. After 6 or 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were: 39, 35, 50, LENALIDOMIDE and POMALIDOMIDE at the concentrations 10 and 20 μM, and a group of compounds listed in the Table 5 at the concentration of 20 μM; the treatment with all compounds was carried out for 24 h. Additionally, compounds 64, 66 and ARV-825 were tested in this assay at the concentrations of 0.1, 1 and 10 μM, for the duration of 6 h. Densitometry values are normalized to the loading control (β-ACTIN) and presented as % of DMSO control, using the following labels:
≤25% for 0-25% of IKZF1 protein reduction,
>25% for 26-74% of IKZF1 protein reduction,
≥75% for 75-100% of IKZF1 protein reduction.

Table 5 shows the list of compounds tested in the IKZF1 degradation assay at the concentration of 20 μM

TABLE 5
The list of compounds used in the IKZF1 degradation
assay at the concentration of 20 μM.

1
3
4
5
6
7
8
15
20
23
24
25
30
36
37

The representative results for compounds: 64, 66 and ARV-825 are shown in FIG. 3 and Table 6. As illustrated in FIG. 3 and Table 6, the compounds of the present invention present no IKZF1 degradation potential, compared to the reference compound ARV-825 which can induce ca 50% of IKZF1 degradation.

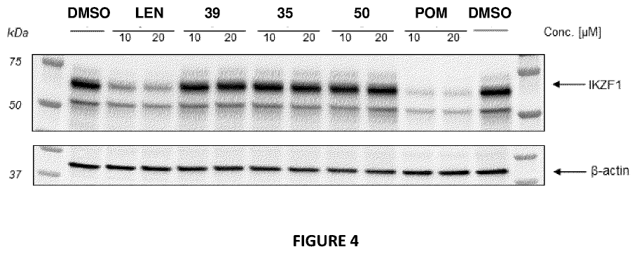

The representative results for compounds: LENALIDOMIDE, 39, 35, 50 and POMALIDOMIDE are shown in FIG. 4 and Table 7. The remaining compounds are presented in Table 10. As illustrated in FIG. 4 and Table 7 and 10, the compounds of the present invention present no IKZF1 degradation capabilities, in contrast to the LENALIDOMIDE and even more effective POMALIDOMIDE.

TABLE 6
IKZF1 degradation in H929 cell line. Cells were treated with the compounds:
64, 66 and ARV-825 at the various concentrations (0.1-10 μM) for 6 h.
% of IKZF1 α protein reduction is provided based on normalized densitometry values.
% of IKZF1 α protein reduction based on densitometry values

| | DMSO | 64 | | | 66 | | | ARV-825 | | |
|---|---|---|---|---|---|---|---|---|---|---|
| [μM] | 0.25% | 10 | 1 | 0.1 | 10 | 1 | 0.1 | 10 | 1 | 0.1 |
| IKZF1 | 0% | ≤25% | ≤25% | ≤25% | ≤25% | ≤25% | ≤25% | >25% | >25% | >25% |

TABLE 7
IKZF1 degradation in H929 cell line. Cells were treated with the compounds:
LENALIDOMIDE, 39, 35, 50 and POMALIDOMIDE at the concentrations 10 and 20 μM for 24 h.
% of IKZF1 α protein reduction is provided based on normalized densitometry values.
% of IKZF1 α protein reduction based on densitometry values

| | DMSO | LENALIDOMIDE | | 39 | | 35 | | 50 | | POMALIDOMIDE | |
|---|---|---|---|---|---|---|---|---|---|---|---|
| [μM] | 0.25% | 10 | 20 | 10 | 20 | 10 | 20 | 10 | 20 | 10 | 20 |
| IKZF1 | 0% | >25% | >25% | ≤25% | ≤25% | ≤25% | ≤25% | ≤25% | ≤25% | ≥75% | ≥75% |

### Example 64: IKZF3 Degradation Assay—H929 Cell Line

The effect of various compounds of the invention and various reference compounds on IKZF3 degradation in the H929 cell line was investigated, using the degradation assay protocol below.

H929 cells were maintained in RPM1-1640 medium, supplemented with penicillin/streptomycin, 10% Fetal Bovine Serum (FBS) and 0.05 mM 2-Mercaptoethanol. Cells were seeded on 6- or 12-well plates, and the compounds to be tested were added at the desired concentration range. Final DMSO concentration was 0.25%. After 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were: LENALIDOMIDE, POMALIDOMIDE, 15, 30, 39, 35 and 50 at the concentrations 10 and 20 μM. The treatment with all compounds was carried out for 24 h. Additionally, compounds 64, 66 and ARV-825 were tested in this assay at the concentrations of 0.1, 1 and 10 μM, for the duration of 6 h. Densitometry values are normalized to the loading control (β-ACTIN) and presented as % of DMSO control, using the following labels:
≤25% for 0-25% of IKZF3 protein reduction,
>25% for 26-74% of IKZF3 protein reduction,
≥75% for 75-100% of IKZF3 protein reduction.

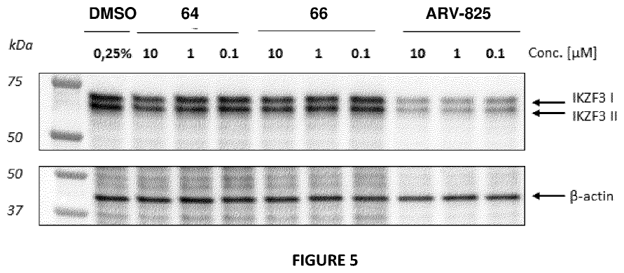

The representative results for compounds: 64, 66 and ARV-825 are shown in FIG. 5 and Table 8. As illustrated in FIG. 5 and Table 8, the compounds of the present invention present no to little IKZF3 degradation potential, compared to the reference compound ARV-825 which shows ca 60% of IKZF3 degradation.

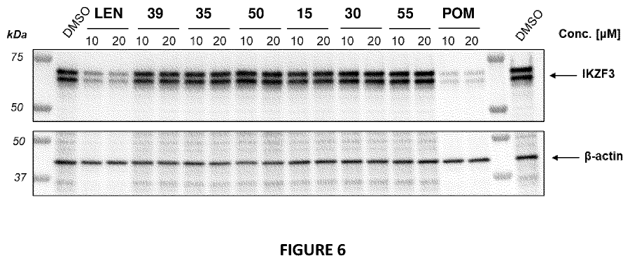

The representative results for compounds: LENALIDOMIDE, 39, 35, 50, 15, 30, 55 and POMALIDOMIDE are shown in FIG. 6 and Table 9. As illustrated in FIG. 6 and Table 9, the compounds of the present invention present no IKZF3 degradation efficiency, in contrast to the LENALIDOMIDE and more potent POMALIDOMIDE.

TABLE 8
IKZF3 degradation in H929 cell line. Cells were treated with the compounds:
64, 66 and ARV-825 at the various concentrations (0.1-10 μM) for 6 h.
% of IKZF3 protein reduction is provided based on normalized densitometry values.
% of IKZF3 protein reduction based on densitometry values

| | DMSO | 64 | | | 66 | | | ARV-825 | | |
|---|---|---|---|---|---|---|---|---|---|---|
| [μM] | 0.25% | 10 | 1 | 0.1 | 10 | 1 | 0.1 | 10 | 1 | 0.1 |
| IKZF3 | 0% | ≤25% | >25% | ≤25% | ≤25% | ≤25% | ≤25% | >25% | >25% | >25% |
| Band I | | | | | | | | | | |
| IKZF3 | 0% | ≤25% | ≤25% | ≤25% | ≤25% | ≤25% | ≤25% | >25% | >25% | >25% |
| Band II | | | | | | | | | | |

TABLE 9
IKZF3 degradation in H929 cell line. Cells were treated
with the compounds: LENALIDOMIDE, 39, 35, 50, 15, 30, 55
and POMALIDOMIDE at the concentrations 10 and 20 μM for 24 h.
% of IKZF3 protein reduction is provided based on normalized densitometry values.
% of IKZF3 protein reduction based on densitometry values

| | DMSO | LENALIDOMIDE | | 39 | | 35 | | 50 | |
|---|---|---|---|---|---|---|---|---|---|
| [μM] | 0.25% | 10 | 20 | 10 | 20 | 10 | 20 | 10 | 20 |
| IKZF3 | 0% | >25% | >25% | ≤25% | ≤25% | ≤25% | ≤25% | ≤25% | ≤25% |
| Band I | | | | | | | | | |
| IKZF3 | 0% | >25% | >25% | ≤25% | ≤25% | ≤25% | ≤25% | ≤25% | ≤25% |
| Band II | | | | | | | | | |

% of IKZF3 protein reduction based on densitometry values

| | 15 | | 30 | | 55 | | POMALIDOMIDE | |
|---|---|---|---|---|---|---|---|---|
| [μM] | 10 | 20 | 10 | 20 | 10 | 20 | 10 | 20 |
| IKZF3 | ≤25% | ≤25% | ≤25% | ≤25% | ≤25% | ≤25% | ≥75% | ≥75% |
| Band I | | | | | | | | |
| IKZF3 | ≤25% | ≤25% | ≤25% | ≤25% | ≤25% | ≤25% | ≥75% | ≥75% |
| Band II | | | | | | | | |

TABLE 10
Summary of Examples 61-65: % of protein
reduction based on densitometry values

| | IKZF 1 | IKZF3 | CK1α | SALL4 |
|---|---|---|---|---|
| Compound | (24 H) | (24 H) | (24 H) | (24 H) |
| 1 | ≤25% | | ≤25% | ≤25% |
| 3 | ≤25% | | ≤25% | ≤25% |
| 4 | ≤25% | | ≤25% | ≤25% |
| 5 | ≤25% | | ≤25% | ≤25% |
| 6 | ≤25% | | ≤25% | ≤25% |
| 7 | ≤25% | | ≤25% | ≤25% |
| 8 | ≤25% | | ≤25% | ≤25% |
| 15 | ≤25% | ≤25% | ≤25% | ≤25% |
| 20 | ≤25% | | ≤25% | ≤25% |
| 23 | ≤25% | | ≤25% | ≤25% |
| 24 | ≤25% | | ≤25% | ≤25% |
| 25 | ≤25% | | ≤25% | ≤25% |
| 30 | ≤25% | ≤25% | ≤25% | ≤25% |
| 35 | ≤25% | ≤25% | ≤25% | ≤25% |
| 36 | ≤25% | | ≤25% | ≤25% |
| 37 | ≤25% | | ≤25% | ≤25% |
| 39 | ≤25% | ≤25% | ≤25% | ≤25% |
| 50 | ≤25% | ≤25% | ≤25% | ≤25% |
| LENALIDOMIDE | >25% | >25% | >25% | ≥75% |
| POMALIDOMIDE | ≥75% | ≥75% | >25% | ≥75% |

### Example 65: BRD4 Degradation Assay—H929 Cell Line

The effect of various compounds of the invention and various reference compounds on BRD4 degradation in the H929 cell line was investigated, using the degradation assay protocol below.

H929 cells were maintained in RPMI-1640 medium (ATCC modified, cat: Gibco A1049101), supplemented with penicillin/streptomycin, 10% Fetal Bovine Serum (FBS) and 0.05 mM 2-Mercaptoethanol. Cells were seeded on 6-well plates (1×10\hat{ }6 cells/condition) and the compounds to be tested were added at the desired concentration range. Final DMSO concentration was 0.25%. After 6 h incubation (37° C., 5% CO₂), cells were harvested, washed and cell lysates were prepared using RIPA lysis buffer. The amount of protein was determined via BCA assay, and the appropriate quantity was then loaded on pre-filled microplate. The analysis was performed using SIMPLE WESTERN™ technology (from Protein Simple), which is an automated, capillary-based immunoassay. The numeric values for the further protein level evaluation process were counted using the software dedicated for Simple Western analysis. Protein normalization is based on the Protein Normalization Reagent by Protein Simple. Numeric values and presented as % of DMSO control, using the following labels:
≤25% for 0-25% of BRD4 protein reduction,
>25% for 26-74% of BRD4 protein reduction,
≥75% for 75-100% of BRD4 protein reduction.

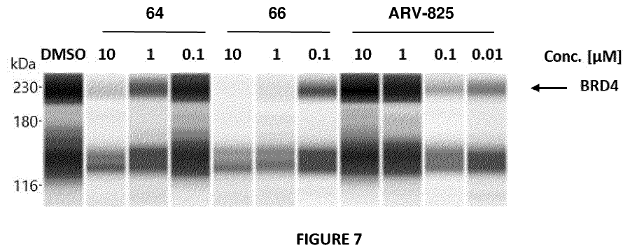

The compounds tested in this assay were: 64, 66 and ARV-825 at the concentrations of 0.1, 1 and 10 μM for 6 h. In addition, ARV-825 was testes at 0.01 μM. The results are shown in FIG. 7 and Table 11. As illustrated in this Figure, the compounds of the present invention have the BRD4 degradation capability.

TABLE 11
BRD4 degradation in H929 cell line. Cells were treated with the compounds:
64, 66 and ARV-825 at the various concentrations (0.1-10 μM) for 6 h.
% of BRD4 protein reduction is provided based on normalized values.
% of BRD4 protein reduction based on normalized values

| | DMSO | 64 | | | 66 | | | ARV-825 | | | |
|---|---|---|---|---|---|---|---|---|---|---|---|
| [μM] | 0.25% | 10 | 1 | 0.1 | 10 | 1 | 0.1 | 10 | 1 | 0.1 | 0.01 |
| BRD4 | 0% | >25% | >25% | ≤25% | ≥75% | ≥75% | >25% | ≤25% | ≤25% | ≥75% | >25% |

### Example 66: BRD4-Compound-CRBN/DDB1 Ternary Complex Formation—AlphaLISA Homogenous Assay

The effect of compounds of the invention on formation of ternary complex composed of BRD4-compound-CRBN/DDB1 was investigated.

Biotinylated BRD4 and His-CRBN/DDB1 complex preparations were centrifugated to remove large aggregates (18000 rcf, 4° C., 5 min). Supernatant was collected and protein concentration was determined spectrophotometrically. The AlphaLISA bead-Protein mixtures were prepared: CRBN-acceptor bead (40 μg/ml Anti-6×His beads, 200 nM His-CRBN/DDB1 in PBS pH 7.4 supplemented with 0.1% Tween-20) and BRD4-donor bead (40 μg/ml Streptavidin beads, 40 nM BRD4 in PBS pH 7.4 supplemented with 0.1% Tween-20 and 2 mM DTT). Bead mixes were incubated in dark for 30 minutes at room temperature. Tested compounds were dispensed into small volume AlphaPlate (Perkin Elmer) using Echo 555 liquid handler.

CRBN-acceptor bead mix and BRD4-donor bead mix were combined and dispensed into plate with compounds and DMSO only (10 μl of master mix per well). Final sample composition: 20 μg/ml Anti-6×His beads, 20 μg/ml Stretavidin beads, 100 nM His-CRBN/DDB1, 20 nM BRD4, 2% DMSO, 0.1% Tween-20, 1 mM DTT in PBS pH 7.4, +/− compound. Plate was sealed and covered to protect against light. Sample was mixed using Vibroturbulator. Subsequently, solutions in the plate was centrifugated and incubated in the dark for 30 minutes at 25° C. The plate seal was removed and sample luminescence was determined using Perkin Elmer Enspire plate reader. Readouts were assigned to certain compound concentration. Solutions without compound were utilized to determine background response (an average value) which subsequently was subtracted from raw data collected for compound mixtures. Results are presented as TF50 values (compound concentration which mediate the half of maximal response observed for Ternary Complex) and AUC (Area Under Curve, which expresses the overall compound potency) values.

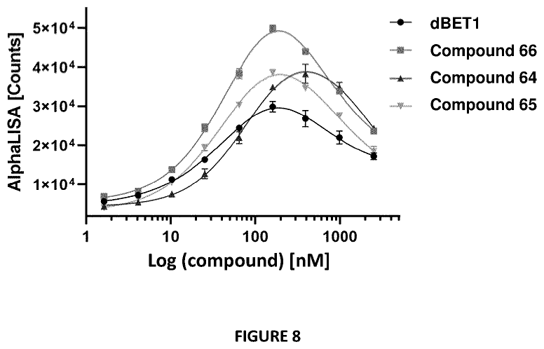

The compounds tested in this assay were: 66, 64, 65, and dBET1. Tested compound concentrations: 1.63, 4.11, 10.3, 25.3, 64.3, 160, 392, 980 and 2500 nM. Results are presented in FIG. 8 and Table 12. As illustrated by this Figure, the bifunctional compounds of the present invention promote BRD4-compound-CRBN/DDB1 was complex formation with high potency.

TABLE 12
The AlphaLISA signal (luminescence) recorded for BRD4-
CRBN/DDB1 TCF in function of compound concentration.
Points present mean with standard deviation (N = 3).

| | TF50 | Normalized AUC |
|---|---|---|
| Compound | [nM] | to dBET1 |
| dBET1 | 27.2 | 1 |
| 64 | 70.5 | 1.45 |
| 65 | 38.8 | 1.22 |
| 66 | 42.1 | 1.53 |

### Example 67: IKZF1-Compound-CRBN/DDB1 Ternary Complex Formation—AlphaLISA Homogenous Assay

The effect of compounds of the invention on formation of ternary complex composed of IKZF1-compound-CRBN/DDB1 was investigated.

Strep-tagged Ikaros (IKZF1 ZF2) and His-CRBN/DDB1 complex preparations were centrifugated to remove large aggregates (18000 rcf, 4° C., 5 min). Supernatant was collected and protein concentration was determined spectrophotometrically. The AlphaLISA bead-Protein mixtures were prepared: CRBN-acceptor bead (40 μg/ml Anti-6×His beads, 200 nM His-CRBN/DDB1 in PBS pH 7.4 supplemented with 0.1% Tween-20) Ikaros-donor bead mix (40 μg/ml Strep-Tactin beads, 800 nM IKZF1 in PBS pH 7.4 supplemented with 0.1% Tween-20 and 2 mM DTT). Bead mixes were incubated in dark for 30 minutes at room temperature. Tested compounds were dispensed into small volume AlphaPlate (Perkin Elmer) using Echo 555 liquid handler. CRBN-acceptor bead mix and Ikaros-donor bead mix were combined and dispensed into plate with compounds and DMSO only (10 μl of master mix per well). Final sample composition: 20 μg/ml Anti-6×His beads, 20 μg/ml Strep-Tactin beads, 100 nM His-CRBN/DDB1, 400 nM IKZF1, 2% DMSO, 0.1% Tween-20, 1 mM DTT in PBS pH 7.4, +/− compound. Plate was sealed and covered to protect against light. Sample was mixed using Vibroturbulator. Subsequently, solutions in the plate was centrifugated and incubated in the dark for 30 minutes at 25° C. The plate seal was removed and sample luminescence was determined using Perkin Elmer Enspire plate reader. Readouts were assigned to certain compound concentration. Solutions without compound were utilized to determine background response (an average value) which subsequently was subtracted from raw data collected for compound mixtures. The average and standard deviation (SD) were calculated for each compound concentration point. Finally, luminescence values were normalized and expressed as % of Lenalidomide response at given concentration (internal, positive control).

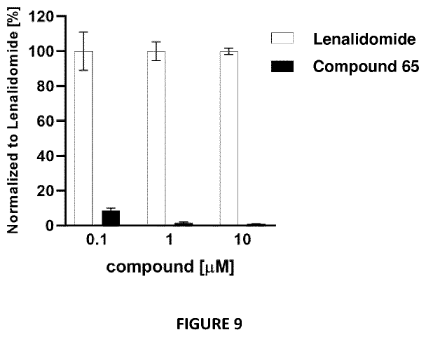

The compounds tested in this assay were: 65 and Lenalidomide. Tested compound concentrations: 0.1, 1 and 10 μM. Results are presented in FIG. 9 (AlphaLISA results from Ikaros-CRBN/DDB1 TCF in the presence of 65, in which the luminescence obtained for mixtures with 65 was normalized to response mediated by Lenalidomide). As illustrated by this Figure, the bifunctional compounds of the present invention do not promote IKZF1-compound-CRBN/DDB1 complex formation.

### SUMMARY

In summary, the presented neosubstrates SALL4, CK1α, IKZF1, IKZF3 degradation test results for the compounds of the present invention show no to low degradation of the proteins by the compounds. This profile gives the compounds the capacity of becoming warheads in bifunctional degraders. Bifunctional compounds 64 and 66 can degrade BRD4 and at the same time are more selective towards substrate degradation.

### Bifunctional Compounds

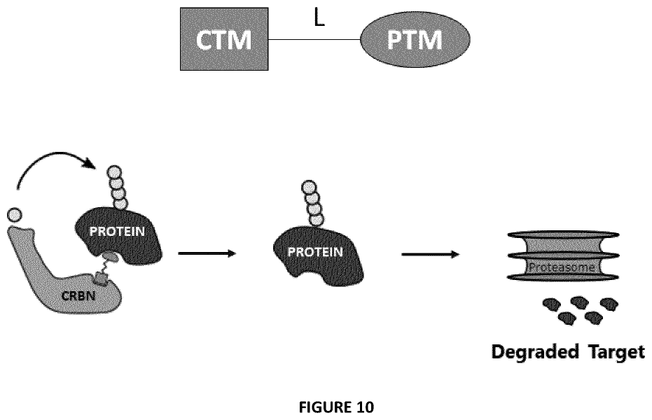

FIG. 10 is a schematic illustration of the general principle for targeted protein degradation upon treatment with a bifunctional compound.

Bifunctional compounds comprise a protein targeting moiety (PTM), a cereblon targeting moiety (CTM), and optionally a linker moiety (L) connecting the PTM to the CTM. A bifunctional compound binds to cereblon (CRBN) ubiquitin ligase at one end, and to the target protein (PROTEIN) at the other end, bringing the target protein into close proximity to cereblon (see bottom left-hand side of FIG. 10). The poly-ubiquitinated protein (shown bottom middle in FIG. 10) is then targeted for degradation by the proteosomal machinery of the cell (see bottom right-hand side of FIG. 10). Examples of linker moieties include those as described in WO2019/199816 and WO2020/010227.

### Abbreviations and Definitions

A list of the abbreviations used in the present application is shown in Table 13, below:

TABLE 13
Abbreviations

| Abbreviation | Meaning |
|---|---|
| CRBN | Cereblon |
| CRL | Cullin RING Ligase |
| CMA | Cereblon Modulating Agent |
| Cy5-T | Cy5-labelled thalidomide |
| DDB1 | damaged DNA binding protein 1 |
| CUL4 | Cullin-4 |
| RBX1 | RING-Box Protein 1 |
| Bn | benzyl |
| Tris | Tris(hydroxymethyl)aminomethane |
| DMSO | Dimethylsulfoxide |
| DMAP | 4-(Dimethylamino)pyridine |
| DIPEA | N,N-Diisopropylethylamine |
| HATU | Hexafluorophosphate Azabenzotriazole |
| | Tetramethyl Uronium: |
| | i.e. 1-[Bis(dimethylamino)methylene]- |
| | 1H-1,2,3-triazolo[4,5-b]pyridinium |
| | 3-oxide hexafluorophosphate |
| CDI | 1,1′-Carbonyldiimidazole |
| EDC | 1-ethyl-3-(3-dimethylaminopropyl)carbodiimide |
| THF | tetrahydrofuran |
| m-CPBA | meta-chloroperbenzoic acid |
| MTBE | methyl tert butyl ether |
| Pd(dppf)Cl₂ | [1,1′- |
| CH₂Cl₂ | bis(diphenylphosphino)ferrocene]dichloropalladium(II), |
| | complex with dichloromethane |
| DTT | dithiothreitol |
| NK cells | Natural killer cells |
| ADCC | antibody-mediated cellular cytotoxicity |
| GVHD | Graft versus host disease |
| HPLC | High performance liquid chromatography |
| TLC | Thin layer chromatograpy |

As used herein, the term “room temperature” means a temperature of between 20° C. and 25° C.

As used herein, the term “small molecule” means an organic compound with a molecular weight of less than 900 Daltons.

### EMBODIMENTS OF THE INVENTION

1. A compound of Formula (I):

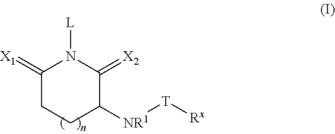

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L is hydrogen, alkyl, alkenyl, aryl, heteroaryl, benzyl, haloalkyl, haloalkenyl, —C(O)H, —C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H or —S(O)₂R″;
R^{x} is selected from

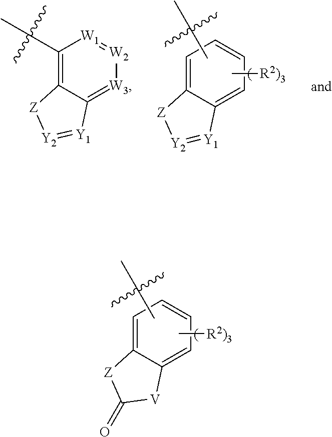

wherein [symbol] indicates attachment to T,
Z is O, S or NH;
V is CR₂, NR⁴ or S;
each of W₁, W₂ and W₃ is independently N or CR²,
each of Y₁ and Y₂ is independently N or CR,
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂;
each R² is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR′2;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H or —S(O)₂R″; and
each R″ is independently alkyl, cycloalkyl, alkenyl, cycloalkenyl, aryl, heteroaryl, or benzyl;
wherein, when R^{x} is

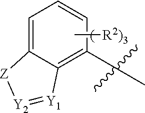

and Z is NH, then n is 1 or 2.
2. The compound of embodiment 1, having the structure:

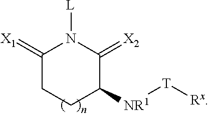

3. The compound of embodiment 1, having the structure:

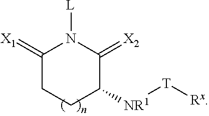

4. The compound of any preceding embodiment, wherein T is C═O.
5. The compound of any one of embodiments 1-3, wherein T is SO₂.
6. The compound of any preceding embodiment, wherein Z is NH.
7. The compound of any one of embodiments 1-5, wherein Z is O.
8. The compound of any one of embodiments 1-5, wherein Z is S.
9. The compound of any preceding embodiment, wherein V is CR₂.
10. The compound of any preceding embodiment, wherein V is NR⁴.
11. The compound of any preceding embodiment, wherein V is S.
12. The compound of any preceding embodiment, wherein Y₁ is N, and Y₂ is CR.
13. The compound of any one of embodiments 1-11, wherein Y₂ is N, and Y₁ is CR.
14. The compound of any one of embodiments 1-11, wherein both of Y₁ and Y₂ are N.
15. The compound of any one of embodiments 1-11, wherein both of Y₁ and Y₂ are CR.
16. The compound of any preceding embodiment, wherein L is hydrogen, alkyl, alkenyl, aryl, heteroaryl, benzyl, —OH, —OR″, —NH₂, —NHR″, —NR″₂, —S(O)₂H or —S(O)₂R″; optionally wherein L is hydrogen, alkyl, alkenyl, aryl, heteroaryl, or benzyl.
17. The compound of embodiment 16, wherein L is hydrogen.
18. The compound of any preceding embodiment, wherein R^{x} is

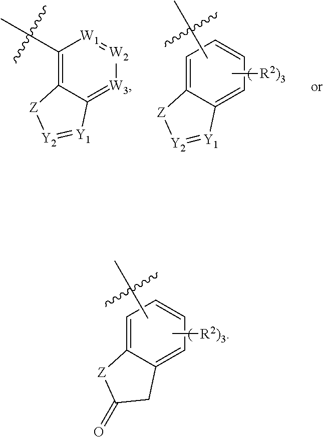

19. The compound of any preceding embodiment, wherein R^{x} is

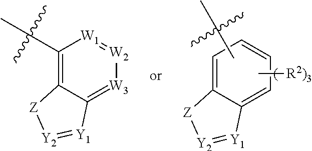

20. The compound of embodiment 19, wherein R^{x} is

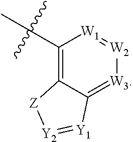

21. The compound of embodiment 20, wherein one of W₁, W₂ and W₃ is N, and the remaining two of W₁, W₂ and W₃ are each CR².
22. The compound of embodiment 20, wherein two of W₁, W₂ and W₃ are N, and the remaining one of W₁, W₂ and W₃ is CR².
23. The compound of embodiment 20, wherein each of W₁, W₂ and W₃ is N.
24. The compound of embodiment 20, wherein each of W₁, W₂ and W₃ is CR².
25. The compound of embodiment 24, wherein:
each R² is hydrogen
Y₁ is N
Y₂ is CH.
   26. The compound of embodiment 25, having the structure:

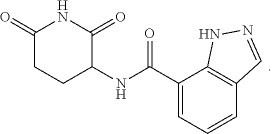 [SMILES: O=C1CCC(NC(=O)c2cccc3cn[nH]c23)C(=O)N1]

27. The compound of embodiment 24, wherein:
each R² is hydrogen,
Y₁ and Y₂ are each CH.
   28. The compound of embodiment 27, having the structure:

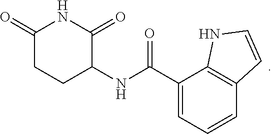 [SMILES: O=C1CCC(NC(=O)c2cccc3cc[nH]c23)C(=O)N1]

29. The compound of embodiment 19, wherein R^{x} is

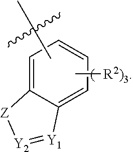

30. The compound of embodiment 29, wherein R^{x} is

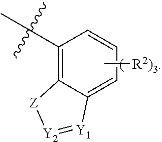

31. The compound of embodiment 29, wherein R^{x} is

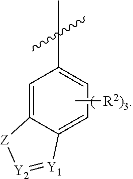

32. The compound of embodiment 29, wherein R^{x} is

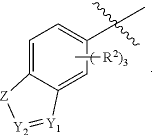

33. The compound of embodiment 29, wherein R^{x} is

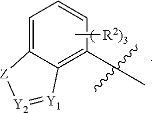

34. The compound of any one of embodiments 1-17, wherein R^{x} is

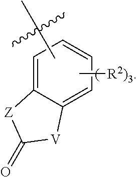

35. The compound of embodiment 34, wherein R^{x} is

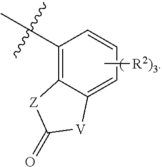

36. The compound of embodiment 34, wherein R^{x} is

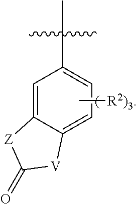

37. The compound of embodiment 34, wherein R^{x} is

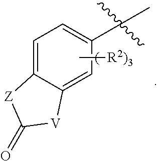

38. The compound of embodiment 34, wherein R^{x} is

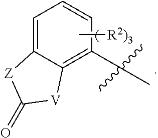

39. The compound of any one of embodiments 34-38, wherein R⁴ is hydrogen, halogen, alkyl, cycloalkyl, alkenyl, cycloalkenyl, alkynyl, aryl, heteroaryl, benzyl, haloalkyl, haloalkenyl, —OH, —OR″, —NH₂, —NHR″, —NR″₂, —S(O)₂H or —S(O)₂R″; optionally wherein R⁴ is hydrogen, halogen, alkyl, cycloalkyl, alkenyl, cycloalkenyl, alkynyl, aryl, heteroaryl, benzyl, haloalkyl, or haloalkenyl.
40. The compound of embodiment 39, wherein R⁴ is hydrogen or alkyl.
41. The compound of any one of embodiments 34-38, wherein V is CH₂.
42. The compound of any one of embodiments 34-41, wherein:
each R² is hydrogen
Z is NH.
   43. The compound of embodiment 34, having the structure:

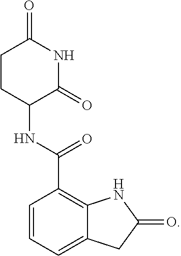 [SMILES: O=C1CCC(NC(=O)c2cccc3c2NC(=O)C3)C(=O)N1]

44. The compound of any preceding embodiment, wherein each R² is independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂; optionally wherein each R² is hydrogen.
45. The compound of any preceding embodiment, wherein each R is independently independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂.
46. The compound of any preceding embodiment, wherein each R is hydrogen.
47. The compound of any preceding embodiment, wherein R¹ is hydrogen.
48. A compound of Formula (II):

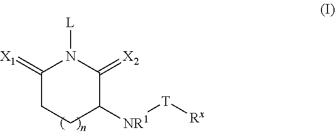

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L is hydrogen, alkyl, alkenyl, aryl, heteroaryl, benzyl, haloalkyl, haloalkenyl, —C(O)H, —C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H or —S(O)₂R″;
R^{y} is selected from

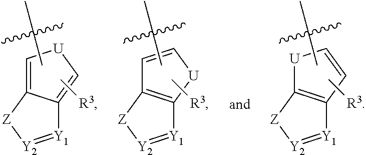

Z is O, S or NR³;
U is O, S, NR³ or CR²²;
each of Y₁ and Y₂ is independently N or CR;
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR′2;
each R² is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR′2;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR′2;
each R″ is independently alkyl, cycloalkyl, alkenyl, cycloalkenyl, aryl, heteroaryl, or benzyl.
   49. The compound of embodiment 48, having the structure:

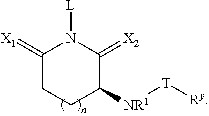

50. The compound of embodiment 48, having the structure:

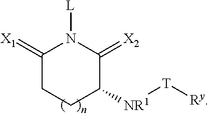

51. The compound of any one of embodiments 48-50, wherein T is C═O.
52. The compound of any one of embodiments 48-50, wherein T is SO₂.
53. The compound of any one of embodiments 48-52, wherein Z is NR³.
54. The compound of any one of embodiments 48-52, wherein Z is O.
55. The compound of any one of embodiments 48-52, wherein Z is S.
56. The compound of any one of embodiments 48-55, wherein Y₁ is N, and Y₂ is CR.
57. The compound of any one of embodiments 48-55, wherein Y₂ is N, and Y₁ is CR.
58. The compound of any one of embodiments 48-55, wherein both of Y₁ and Y₂ are N.
59. The compound of any one of embodiments 48-55, wherein both of Y₁ and Y₂ are CR.
60. The compound of any one of embodiments 48-59, wherein L is hydrogen, alkyl, alkenyl, aryl, heteroaryl, benzyl, —OH, —OR″, —NH₂, —NHR″, —NR″₂, —S(O)₂H or —S(O)₂R″; optionally wherein L is hydrogen, alkyl, alkenyl, aryl, heteroaryl, or benzyl.
61. The compound of embodiment 60, wherein L is hydrogen.
62. The compound of any one of embodiments 48-61, wherein R^{y} is

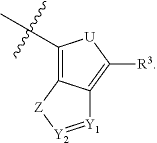

63. The compound of any one of embodiments 48-61, wherein R^{y} is

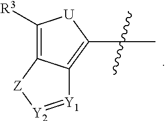

64. The compound of any one of embodiments 48-61, wherein R^{y}

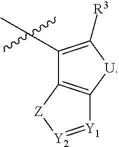

65. The compound of any one of embodiments 48-61, wherein R^{y} is

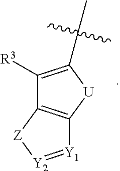

66. The compound of any one of embodiments 48-61, wherein R″ is

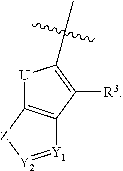

67. The compound of any one of embodiments 48-61, wherein R″ is

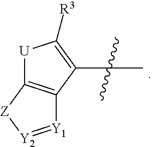

68. The compound of any one of embodiments 48-67, wherein each R² is independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂.
69. The compound of any one of embodiments 48-68, wherein each R² is hydrogen.
70. The compound of any one of embodiments 48-69, wherein each R is independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, or —S(O)₂NR″₂.
71. The compound of any one of embodiments 48-70, wherein each R is hydrogen.
72. The compound of any one of embodiments 48-71, wherein each R³ is independently hydrogen, alkyl, cycloalkyl, alkenyl, cycloalkenyl, alkynyl, aryl, heteroaryl, benzyl, haloalkyl, haloalkenyl, or C(O)R″.
73. The compound of any one of embodiments 48-72, wherein each R³ is hydrogen
74. The compound of any one of embodiments 48-74, wherein R¹ is hydrogen.
75. The compound of any preceding embodiment, wherein X₁ and X₂ are O.
76. The compound of any one of embodiments 1-74, wherein X₁ is O and X₂ is S.
77. The compound of any one of embodiments 1-74, wherein X₁ is S and X₂ is O.
78. The compound of any one of embodiments 1-74, wherein X₁ and X₂ are S.
79. The compound of any preceding embodiment, wherein n is 0.
80. The compound of any one of embodiments 1-78, wherein n is 1.
81. The compound of any one of embodiments 1-78, wherein n is 2.
82. A compound of any one of the preceding embodiments, for use as a cereblon binder.
83. A pharmaceutical composition comprising a compound of any one of embodiments 1-81.
84. A compound of any one of embodiments 1-81, or a composition according to embodiment 83, for use in medicine.
85. A compound of any one of embodiments 1-81, or a composition according to embodiment 83, for use in immune-oncology.
86. A compound of any one of embodiments 1-81, or a composition according to embodiment 83, for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.
87. A method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders;
wherein the method comprises administering to a patient in need thereof an effective amount of compound of any one of embodiments 1-81 or a composition according to embodiment 83.
   88. The method of embodiment 87, further comprising administering at least one additional active agent to the patient.
   89. A combined preparation of a compound of any one of embodiments 1-81 and at least one additional active agent, for simultaneous, separate or sequential use in therapy.
   90. The combined preparation of embodiment 89, or the method of embodiment 88, wherein the at least one additional active agent is an anti-cancer agent or an agent for the treatment of an autoimmune disease.
   91. The combined preparation of any one of embodiments 89-90, or the method of embodiment 88 or 90, wherein the at least one additional active agent is a small molecule, peptide, an antibody, a corticosteroid, or a combination thereof.
   92. The combined preparation or method of embodiment 91, wherein the at least one additional active agent is at least one of bortezomib, dexamethasone, and rituximab.
   93. The combined preparation of any one of embodiments 89-92, wherein the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

1-115. (canceled)

116. A compound of Formula (I): 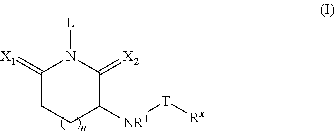
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 1 or 2;
L is hydrogen, alkenyl, aryl, heteroaryl, benzyl, haloalkyl, haloalkenyl, —C(O)H, —C(O)R″, —C(O)OH, —C(O)OR″, —CH₂C(O)OR″, —C(O)NH₂, —C(O)NHR″, —C(O)NR″₂, —OH, —OR″, —NH₂, —NR″, —NR″₂, —S(O)₂H, or —S(O)₂R″;
R^{x} is 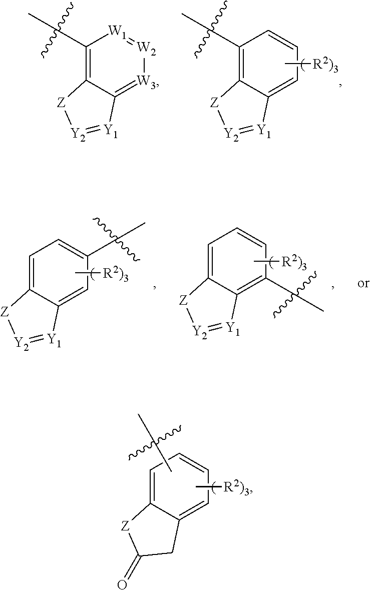
wherein Z is O, S or NR⁴;
or R^{x} is 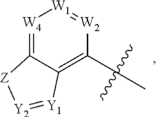
wherein Z is S or NR⁴;
wherein [symbol] indicates attachment to T;
V is CR₂, NR⁴, or S;
each of W₁, W₂, W₃, and W₄ is independently N or CR²;
each of Y₁ and Y₂ is independently N or CR;
each R is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, —C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂₀R″, —S(O)₂NH₂, —S(O)₂NHR″, or —S(O)₂NR″₂; or when Y₁ and Y₂ are CR then each R, together with the carbon atom to which it is attached, forms a 5- or 6-membered ring;
each R² is independently hydrogen, halogen, alkyl, cycloalkyl, alkenyl, cycloalkenyl, alkynyl, aryl, aryl substituted with at least one —OR″, heteroaryl, benzyl, haloalkyl, haloalkenyl, —NH₂, —NHR″, —NR″₂, —CH₂NH₂, —NHC(O)R″, —NR″C(O)R″, —NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂₀R″, —S(O)₂NH₂, —S(O)₂NHR″, or —S(O)₂NR″₂;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, —C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H, or —S(O)₂R″; and
each R″ is independently alkyl, cycloalkyl, alkenyl, cycloalkenyl, aryl, heteroaryl, or benzyl;
wherein, when n=2, each R² is hydrogen, and each of W₁, W₂, W₃ and W₄ is CR², then C═X₁ is replaced by CH₂;
and wherein:
(i) when R^{x} is 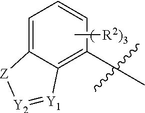
and Z is NH, then L is hydrogen, —CH₂C(O)OR″, or —OR″;
(ii) when R^{x} is 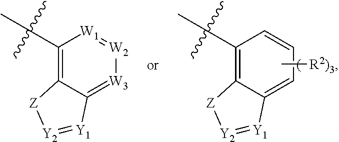
Z is NR⁴, Y₁ is CR, and Y₂ is N, then R⁴ is not alkyl and at least one of R² and R is not H;
(iii) when R^{x} is 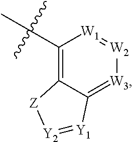
Z is NR⁴, and Y₁ and Y₂ are CR, then at least one of W₁, W₂ and W₃ is N;
(iv) when Z is NR⁴, and Y₁ and Y₂ are CR, then R^{x} is not 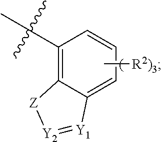
(v) when R^{x} is 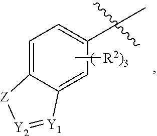
Z is NR⁴, and Y₁ or Y₂ is N, then R⁴ is not alkyl; and
(vi) when R^{x} is 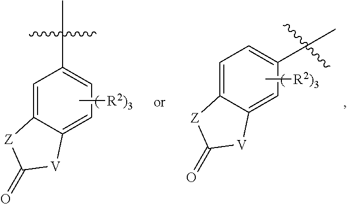
then Z═O or S.

117. The compound of claim 116, wherein T is C═O.

118. The compound of claim 116, wherein V is CR² or NR⁴.

119. The compound of claim 116, wherein L is hydrogen, —CH₂C(O)OR″, or —OR″.

120. The compound of claim 116, wherein R^{x} is

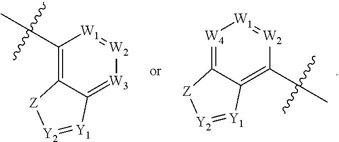

121. The compound of claim 120, wherein one of W₁, W₂, and W₄ is N, and the remaining two of W₁, W₂, and W₄ are each CR².

122. The compound of claim 120, wherein each of W₁, W₂, and W₃ is CR².

123. The compound of claim 122, having the structure:

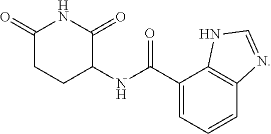 [SMILES: O=C1CCC(NC(=O)c2cccc3nc[nH]c23)C(=O)N1]

124. The compound of claim 116, wherein R^{x} is

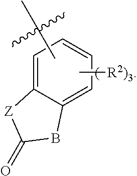

125. The compound of claim 124, having the structure:

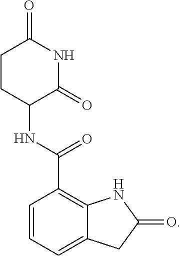 [SMILES: O=C1CCC(NC(=O)c2cccc3c2NC(=O)C3)C(=O)N1]

126. The compound of claim 116, wherein each R² is independently hydrogen, halogen, alkyl, heteroaryl, —NH₂, —NHR″, —NHC(O)R″, —NHSO₂R″, —CN, —C(O)NH₂, —C(O)NHR″, —C(O)NR″₂, —OH, —OR″, —S(O)₂NH₂, —S(O)₂NHR″, —S(O)₂NR″₂, —CH₂NH₂, —NO₂, or aryl substituted with at least one —OR″.

127. The compound of claim 116, wherein the compound is:

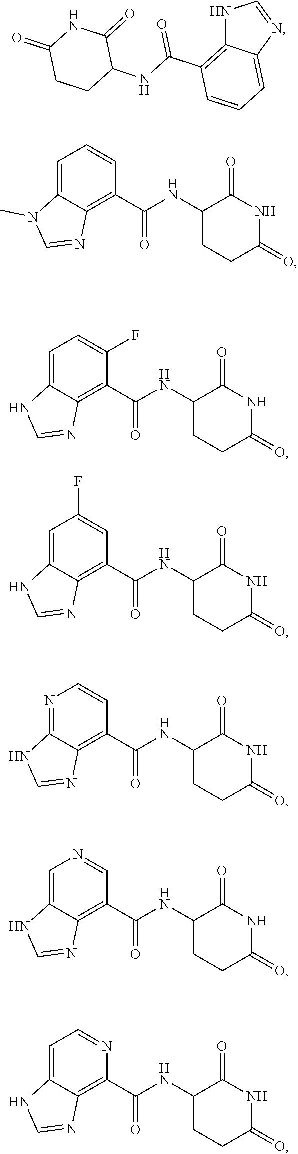

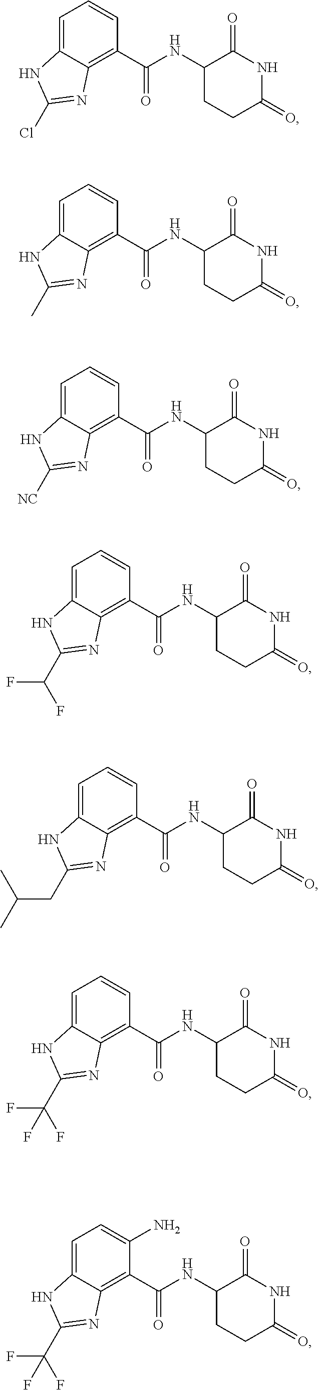

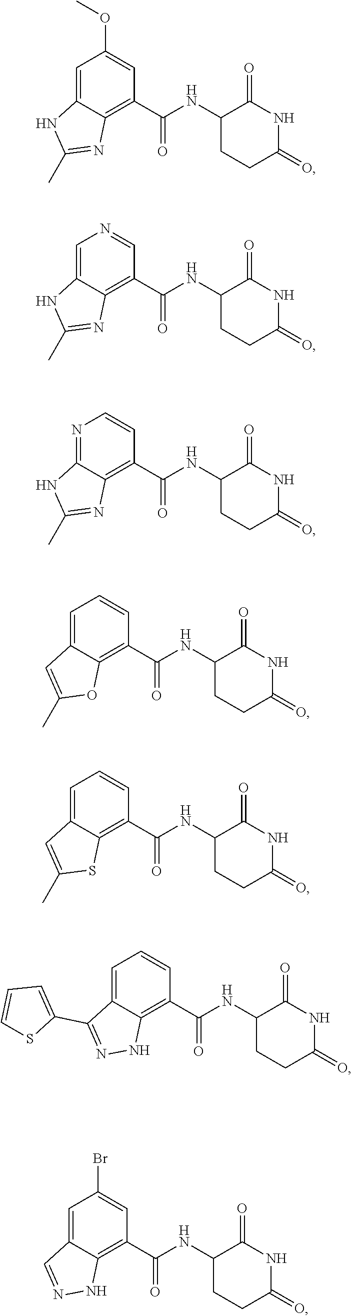

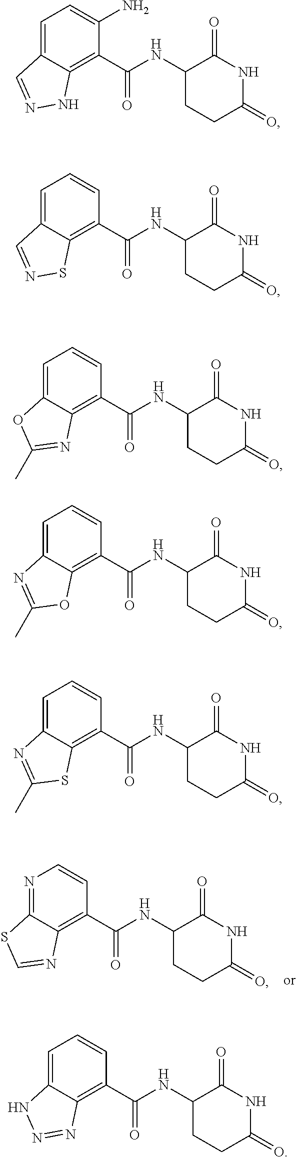

128. A compound of Formula (II): 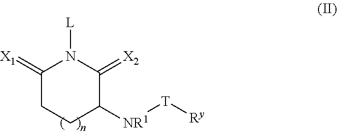
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1, or 2;
L is hydrogen, alkyl, alkenyl, aryl, heteroaryl, benzyl, haloalkyl, haloalkenyl, —C(O)H, —C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H, or —S(O)₂R″;
R^{y} is 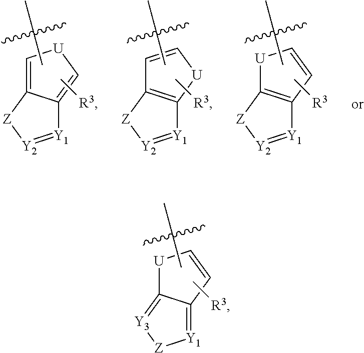
wherein [symbol] indicates attachment to T,
Z is O, S, or NR³;
U is O, S, NR³, or CR²2;
each of Y₁, Y₂, and Y₃ is independently N or CR;
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, —NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, —C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂;
each R² is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, —NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, —NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂₀R″, —S(O)₂NH₂, —S(O)₂NHR″, or —S(O)₂NR″₂;
each R″ is independently alkyl, cycloalkyl, alkenyl, cycloalkenyl, aryl, heteroaryl, or benzyl;
wherein,
(i) when R^{y} is 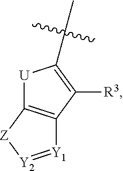
then Y₂ is CR; and
(ii) when R^{y} is 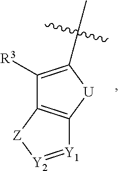
then R³ is not hydrogen.

129. The compound of claim 128, wherein L is hydrogen, alkyl, alkenyl, aryl, heteroaryl, benzyl, —OH, —OR″, —NH₂, —NHR″, —NR″₂, —S(O)₂H, or —S(O)₂R″.

130. The compound of claim 128, wherein R^{y} is

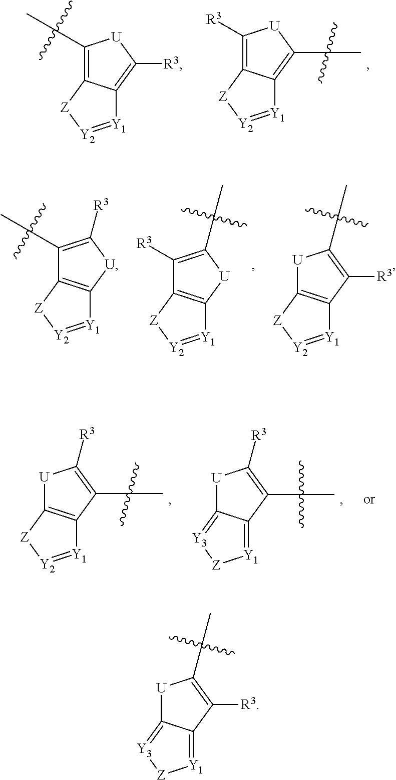

131. The compound of claim 128, wherein the compound is:

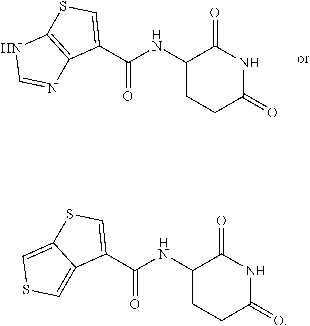 [SMILES: O=C1CCC(NC(=O)c2csc3[nH]cnc23)C(=O)N1 ; O=C1CCC(NC(=O)c2csc3cscc23)C(=O)N1]

132. A bifunctional compound having the structure:
CLM-L′-PTM,
or a pharmaceutically acceptable salt, enantiomer, stereoisomer, solvate, polymorph or prodrug thereof, wherein:
CLM is a cereblon E3 ubiquitin ligase binding moiety;
PTM is a protein targeting moiety; and
L′ is a bond or a chemical linking moiety covalently coupling the CLM and the PTM; and
wherein the CLM is a compound of claim 116, wherein at least one of R, R², R³, and R⁴ is covalently attached to L′ or to the PTM.

133. The bifunctional compound of claim 132, wherein L′ is: 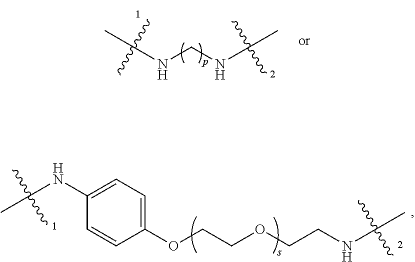
wherein [symbol]
indicates attachment to the PTM, and [symbol] indicates attachment to the CLM;
p is an integer from 3 to 12; and
s is an integer from 1 to 6.

134. The bifunctional compound of claim 133, wherein L′ is:

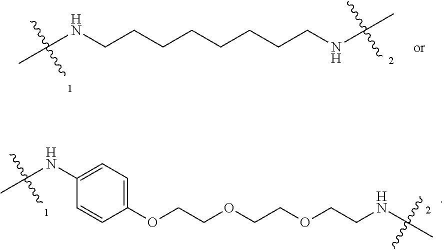 [SMILES: *NCCCCCCCCN* ; *NCCOCCOCCOc1ccc(N*)cc1]

135. The bifunctional compound of claim 132, wherein the PTM is 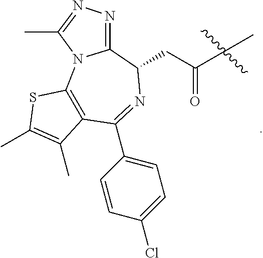 [SMILES: *C(=O)C[C@@H]1N=C(c2ccc(Cl)cc2)c2c(sc(C)c2C)-n2c(C)nnc21]
wherein [symbol] indicates attachment to L′.

136. The bifunctional compound of claim 132, wherein at least one of R, R², R³, and R⁴ comprises a carboxylic acid or an ester.

137. The bifunctional compound of claim 132, wherein the compound is 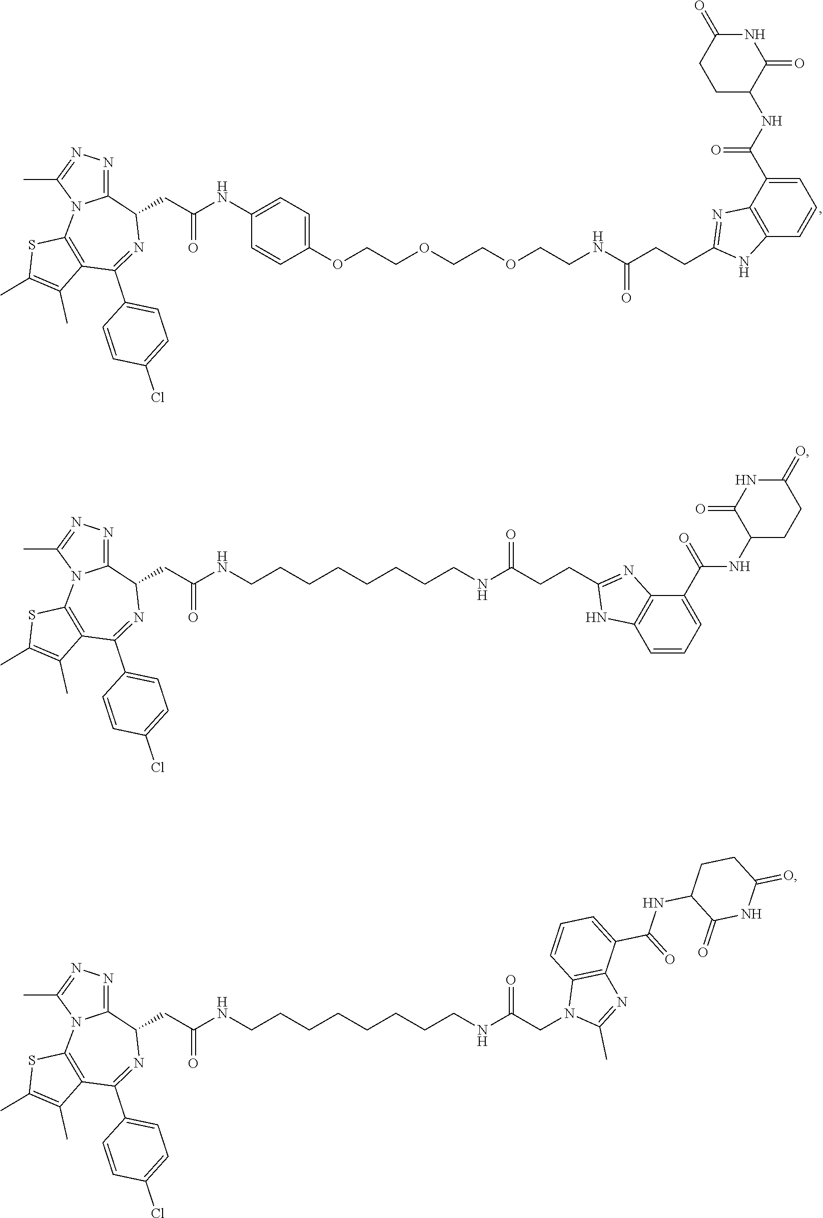 [SMILES: Cc1sc2c(c1C)C(c1ccc(Cl)cc1)=N[C@@H](CC(=O)Nc1ccc(OCCOCCOCCNC(=O)CCc3nc4c(C(=O)NC5CCC(=O)NC5=O)cccc4[nH]3)cc1)c1nnc(C)n1-2 ; Cc1sc2c(c1C)C(c1ccc(Cl)cc1)=N[C@@H](CC(=O)NCCCCCCCCNC(=O)CCc1nc3c(C(=O)NC4CCC(=O)NC4=O)cccc3[nH]1)c1nnc(C)n1-2 ; Cc1sc2c(c1C)C(c1ccc(Cl)cc1)=N[C@@H](CC(=O)NCCCCCCCCNC(=O)Cn1c(C)nc3c(C(=O)NC4CCC(=O)NC4=O)cccc31)c1nnc(C)n1-2] 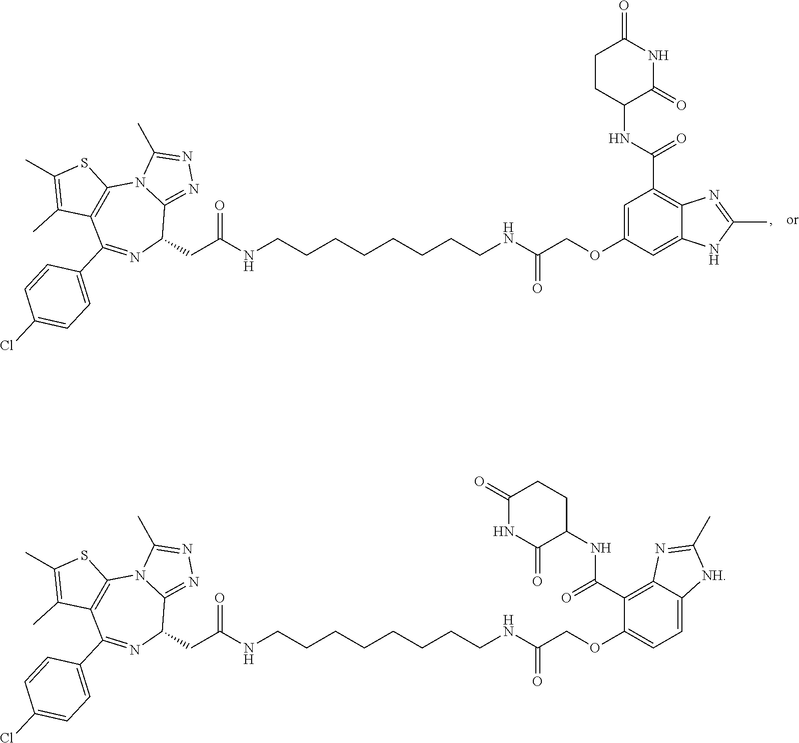 [SMILES: Cc1nc2c(C(=O)NC3CCC(=O)NC3=O)cc(OCC(=O)NCCCCCCCCNC(=O)C[C@@H]3N=C(c4ccc(Cl)cc4)c4c(sc(C)c4C)-n4c(C)nnc43)cc2[nH]1 ; Cc1nc2c(C(=O)NC3CCC(=O)NC3=O)c(OCC(=O)NCCCCCCCCNC(=O)C[C@@H]3N=C(c4ccc(Cl)cc4)c4c(sc(C)c4C)-n4c(C)nnc43)ccc2[nH]1]
